(19) United States
(12) Patent Application Publication
(10) Pub. No.: US 2009/0018133 A1
(43) Pub. Date: Jan. 15, 2009

(54) Novel Adamantane Derivatives

(76) Inventors: Rhonan Ford, Loughborough (GB); Frederic Leroux, Saint Louis (FR); Michael Stocks, Loughborough (GB)

Correspondence Address: FISH & RICHARDSON P.C., P.O BOX 1022, MINNEAPOLIS, MN, 55440-1022, US

(21) Appl. No.: 12/040,462

(22) Filed: Feb. 29, 2008

Related U.S. Application Data
(63) Continuation of application No. 10/505,789, filed on Aug. 25, 2004, now abandoned.

(30) Foreign Application Priority Data
Mar. 25, 2002 (SE) 0200920-7

(51) Int. Cl. A61K 31/5377; C07D 215/38; A61K 31/47; C07D 413/12; A61P 11/00

(52) U.S. Cl. 514/235.2; 546/171; 514/311; 514/313; 546/159; 544/128

(57) ABSTRACT

The invention provides compounds of formula (I), in which m, A, R¹ and Ar have the meanings defined in the specification; processes for their preparation; pharmaceutical compositions containing them; a process for preparing the pharmaceutical compositions; and their use in therapy.

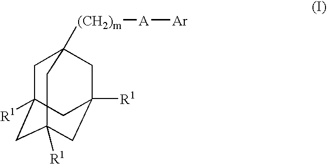

The present invention relates to adamantane derivatives, processes for their preparation, pharmaceutical compositions containing them, a process for preparing the pharmaceutical compositions, and their use in therapy.

The P2X₇ receptor (previously known as P2Z receptor), which is a ligand-gated ion channel, is present on a variety of cell types, largely those known to be involved in the inflammatory/immune process, specifically, macrophages, mast cells and lymphocytes (T and B). Activation of the P2X₇ receptor by extracellular nucleotides, in particular adenosine triphosphate, leads to the release of interleukin-1β (IL-1β) and giant cell formation (macrophages/microglial cells), degranulation (mast cells) and proliferation (T cells), apoptosis and L-selectin shedding (lymphocytes). P2X₇ receptors are also located on antigen-presenting cells (APC), keratinocytes, salivary acinar cells (parotid cells), hepatocytes and mesangial cells.

It would be desirable to make compounds effective as P2X₇ receptor antagonists for use in the treatment of inflammatory, immune or cardiovascular diseases, in the aetiologies of which the P2X₇ receptor may play a role.

In accordance with the present invention, there is therefore provided a compound of formula

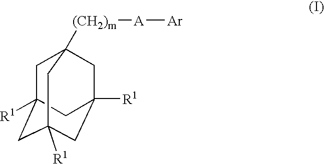

wherein m represents 1, 2 or 3, preferably 1 or 2;
each R¹ independently represents a hydrogen or halogen (e.g. fluorine, chlorine, bromine or iodine) atom, preferably a hydrogen atom;
A represents C(O)NH or NHC(O);
Ar represents a group of formula

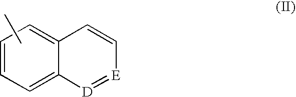

in which one of D and E represents a nitrogen atom and the other of D and E represents CH, the group of formula (II) being optionally substituted by one or more substituent groups R² independently selected from halogen, C₁-C₆ alkyl optionally substituted by at least one substituent selected from hydroxyl, halogen and C₁-C₆ alkoxy, or a group of formula

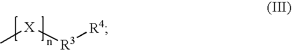

X represents an oxygen or sulphur atom or a group >N—R;
n is 0 or 1;
R³ represents a bond or a C₁-C₈ alkyl group which may be optionally substituted by at least one substituent selected from hydroxyl, halogen, C₁-C₆ alkoxy, C₁-C₆ alkylthio, C₁-C₆ hydroxyalkyl, C₁-C₆ hydroxyalkyloxy, C₁-C₆ alkoxycarbonyl, C₃-C₈ cycloalkyl, phenyl (optionally substituted by at least one substituent selected from halogen, hydroxyl and C₁-C₆ alkylsulphonylamino), benzyl, indolyl (optionally substituted by at least one substituent selected from C₁-C₆ alkoxy), oxopyrrolidinyl, phenoxy, benzodioxolyl, phenoxyphenyl, piperidinyl and benzyloxy;
R⁴ represents hydrogen, hydroxyl or a group —NR⁶R⁷ except that when R³ represents a bond, then R⁴ represents a saturated or unsaturated 4- to 9-membered ring system which may comprise at least one ring heteroatom selected from nitrogen, oxygen and sulphur, the ring system being optionally substituted by at least one substituent selected from hydroxyl, amino (—NH₂), C₁-C₆ alkyl, C₁-C₆ alkylamino, —NH(CH₂)₂OH, —NH(CH₂)₃OH, C₁-C₆ hydroxyalkyl, benzyl and

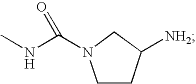

R⁵ represents a hydrogen atom or a C₁-C₅ alkyl group which may be optionally substituted by at least one substituent selected from hydroxyl, halogen and C₁-C₆ alkoxy;
R⁶ and R⁷ each independently represent hydrogen, pyrrolidinyl, C₁-C₆ alkylcarbonyl, C₂-C₇ alkenyl, or C₁-C₇ alkyl optionally substituted with at least one substituent selected from carboxyl, hydroxyl, amino (—NH₂), C₁-C₆ alkylamino, di-C₁-C₆ alkylamino, —NH(CH₂)₂OH, C₁-C₆ alkoxy, C₁-C₆ alkylthio, C₁-C₆ alkoxycarbonyl, and a saturated or unsaturated 3- to 10-membered ring system which may comprise at least one ring heteroatom selected from nitrogen, oxygen and sulphur, the ring system being optionally substituted by at least one substituent selected from halogen, hydroxyl, oxo, carboxyl, cyano, C₁-C₆ alkyl, C₁-C₆ hydroxyalkyl, —NR⁸R⁹, —(CH₂)ᵣNR¹⁰R¹¹ and —CONR¹²R¹³,
or R⁶ and R⁷ may together with the nitrogen atom to which they are attached form a saturated six-membered heterocyclic ring which may comprise a second ring heteroatom selected from nitrogen and oxygen, the ring being optionally substituted by at least one is substituent selected from hydroxyl, halogen, C₁-C₆ allyl and C₁-C₆ hydroxyalkyl;
r is 1, 2, 3, 4, 5 or 6;
R⁸ and R⁹ each independently represent a hydrogen atom or a C₁-C₆ alkyl, C₂-C₆ hydroxyalkyl or C₃-C₈ cycloalkyl group, or R⁸ and R⁹ together with the nitrogen atom to which they are attached form a 3- to 8-membered saturated heterocyclic ring;
R¹⁰ and R¹¹ each independently represent a hydrogen atom or a C₁-C₆ alkyl, C₂-C₆ hydroxyalkyl or C₃-C₈ cycloalkyl group, or R¹⁰ and R¹¹ together with the nitrogen atom to which they are attached form a 3- to 8-membered saturated heterocyclic ring; and
R¹² and R¹³ each independently represent a hydrogen atom or a C₁-C₆ alkyl, C₂-C₆ hydroxyalkyl or C₃-C₈ cycloalkyl group, or R¹² and R¹³ together with the nitrogen atom to which they are attached form a 3- to 8-membered saturated heterocyclic ring;
with the proviso that the compound of formula (I) is not N-(tricyclo[3.3.1.1^{3,7}]dec-1-ylmethyl)-2-quinolinecarboxamide, or 2-(2-thienyl)-N-(tricyclo[3.3.1.1^{3,7}]dec-1-ylmethyl)-4-quinolinecarboxamide;
or a pharmaceutically acceptable salt or solvate thereof.

In one embodiment, the invention provides a compound of formula (I) or a pharmaceutically acceptable salt or solvate thereof as defined above, with the proviso that the compound of formula (I) is not one of the following compounds:
N-(tricyclo[3.3.1.1^{3,7}]dec-1-ylmethyl)-2-quinolinecarboxamide,
2-(2-thienyl)-N-(tricyclo[3.3.1.1^{3,7}]dec-1-ylmethyl)-4-quinolinecarboxamide,
N-(2-methyl-4-quinolinyl)-tricyclo[3.3.1.1^{3,7}]decane-1-acetamide,
2-phenyl-N-(tricyclo[3.3.1.1^{3,7}]dec-1-ylmethyl)-4-quinolinecarboxamide, and
N-(tricyclo[3.3.1.1° ]dec-1-ylmethyl)-4-quinolinecarboxamide.

In the context of the present specification, unless otherwise indicated, an alkyl substituent or alkyl moiety in a substituent group may be linear or branched. Examples of alkyl groups/moieties containing up to 7 carbon atoms include methyl, ethyl, n-propyl, isopropyl, n-butyl, isobutyl, tert-butyl, n-pentyl, n-hexyl and n-heptyl. In formula (III), any hydroxyl groups will not normally be attached to a carbon atom adjacent a nitrogen atom. Further, when R³ is other than a bond, the group R⁴ may be attached to the C₁-C₅ alkyl moiety of R³ at any suitable point; thus R⁴ may be attached to an internal or terminal carbon atom of the C₁-C₅ alkyl moiety of R³. Also, it should be understood that the group of formula (II) may be attached to the group A through any one of the ring carbon atoms but not the nitrogen atom. A hydroxyalkyl substituent may contain one or more hydroxyl groups but preferably contains one hydroxyl group.

Examples of the group Ar that may advantageously be used include:

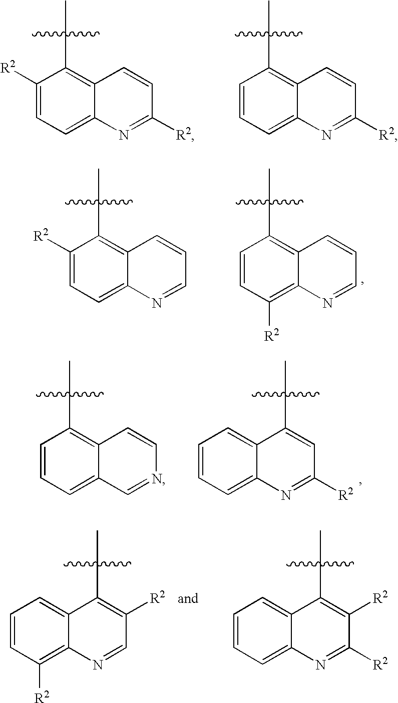

where R² is as defined above.

In an embodiment of the invention, Ar is

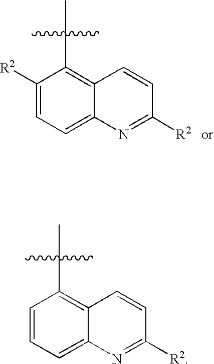

R³ represents a bond or a C₁-C₅ alkyl group which may be optionally substituted by at least one substituent (e.g. one, two or three substituents independently) selected from hydroxyl, halogen (e.g. fluorine, chlorine, bromine or iodine), C₁-C₆, or C₁-C₄, alkoxy, C₁-C₆, or C₁-C₄, alkylthio, C₁-C₆, or C₁-C₄, hydroxyalkyl, C₁-C₆, or C₁-C₄, hydroxyalkyloxy, C₁-C₆, or C₁-C₄, alkoxycarbonyl, C₃-C₈ cycloalkyl, phenyl (optionally substituted by at least one substituent, e.g. one, two or three substituents independently, selected from halogen, hydroxyl and C₁-C₆, or C₁-C₄, alkylsulphonylamino), benzyl, indolyl (optionally substituted by at least one substituent, e.g. one, two or three substituents independently, selected from C₁-C₆, or C₁-C₄, alkoxy), oxopyrrolidinyl, phenoxy, 1,3-benzodioxolyl, phenoxyphenyl, piperidinyl and benzyloxy.

In an embodiment of the invention, R³ represents a bond or a C₁-C₄ alkyl group which may be optionally substituted by one, two or three substituents independently selected from hydroxyl, C₁-C₂ alkoxy, methylthio, C₁-C₂ hydroxyalkyl, C₁-C₂ hydroxyalkyloxy, methoxycarbonyl, C₃-C₆ cycloalkyl, phenyl (optionally substituted by at least one substituent selected from halogen, hydroxyl and methylsulphonylamino), benzyl, indolyl (optionally substituted by at least one methoxy), oxopyrrolidinyl, phenoxy, benzodioxolyl, phenoxyphenyl, piperidinyl and benzyloxy.

In another embodiment of the invention, R³ represents a bond or a C₁-C₄ alkyl group which may be optionally substituted by one, two or three substituents independently selected from hydroxyl, C₁-C₂ alkoxy, methylthio, C₁-C₂ hydroxyalkyl, C₁-C₂ hydroxyalkyloxy, methoxycarbonyl, cyclopropyl, phenyl (optionally substituted by at least one substituent selected from chlorine, hydroxyl and methylsulphonylamino), benzyl, indolyl (optionally substituted by at least one methoxy), oxopyrrolidinyl, phenoxy, benzodioxolyl, phenoxyphenyl, piperidinyl and benzyloxy.

R⁴ represents hydrogen, hydroxyl or a group —NR⁶R⁷ except that when R³ represents a bond, then R⁴ represents a saturated or unsaturated 4- to 9-membered ring system which may comprise at least one ring heteroatom (e.g. one, two, three or four ring heteroatoms independently) selected from nitrogen, oxygen and sulphur, the ring system being optionally substituted by at least one substituent (e.g. one, two, three or four substituents independently) selected from hydroxyl, amino (—NH₂), C₁-C₆, or C₁-C₄, alkyl, C₁-C₆, or C₁-C₄, alkylamino, —NH(CH₂)₂OH, —NH(CH₂)₃OH, C₁-C₆, or C₁-C₄, hydroxyalkyl, benzyl and

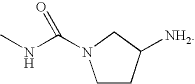

In an embodiment of the invention, R⁴ represents hydrogen, hydroxyl or a group —NR⁶R⁷ except that when R³ represents a bond, then R⁴ represents a saturated or unsaturated 4- to 9-membered ring system which may comprise one or two ring heteroatoms independently selected from nitrogen, oxygen and sulphur, the ring system being optionally substituted by at least one substituent (e.g. one, two, three or four substituents independently) selected from hydroxyl, amino (—NH₂), C₁-C₂ alkyl, C₁-C₂ alkylamino, —NH(CH₂)₂OH, —NH(CH₂)₃OH, C₁-C₂ hydroxyalkyl, benzyl and

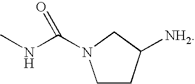

In another embodiment of the invention, R⁴ represents hydrogen, hydroxyl or a group —NR⁶R⁷ except that when R³ represents a bond, then R⁴ represents a saturated or unsaturated 4- to 9-membered ring system which may comprise one or two ring nitrogen atoms, the ring system being optionally substituted by one or two substituents independently selected from hydroxyl, amino (—NH₂), methyl, C₁-C₂ alkylamino, —NH(CH₂)₂OH, —NH(CH₂)₃OH, C₁-C₂ hydroxyalkyl, benzyl and

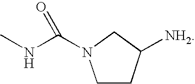

When R⁴ represents a saturated or unsaturated 4- to 9-membered ring system, the ring system may be monocyclic or polycyclic (e.g. bicyclic) and may have alicyclic or aromatic properties. An unsaturated ring system will be partially or fully unsaturated. Examples of ring systems that may be used include cyclobutyl, cyclopentyl, cyclohexyl, cycloheptyl, cyclopentenyl, cyclohexenyl, bicyclo[2.2.1]hept-2-yl, bicyclo[2.2.1]hept-5-en-2-yl, 2,3-dihydro-1H-indenyl, phenyl, pyrrolidinyl, piperidinyl, piperazinyl, pyrazolyl, thiazolidinyl, indanyl, thienyl, isoxazolyl, thiadiazolyl, pyrrolyl, furyl, thiazolyl, indolyl, imidazolyl, benzimidazolyl, triazolyl, tetrazolyl and pyridinyl.

In one embodiment of the invention, the saturated or unsaturated 4- to 9-membered ring system is selected from cyclobutyl, cyclohexyl, bicyclo[2.2.1]hept-2-yl, 2,3-dihydro-1H-indenyl, pyrrolidinyl, piperidinyl and piperazinyl.

R⁵ represents a hydrogen atom or a C₁-C₅ alkyl group which may be optionally substituted by at least one substituent (e.g. one, two or three substituents independently) selected from hydroxyl, halogen (e.g. fluorine, chlorine, bromine or iodine) and C₁-C₆, or C₁-C₄, alkoxy.

In an embodiment of the invention, R⁵ represents a hydrogen atom or a C₁-C₅ alkyl group which may be optionally substituted by at least one hydroxyl group.

In one embodiment of the invention, R⁶ and R⁷ each independently represent hydrogen, pyrrolidinyl, C₁-C₆, or C₁-C₄, alkylcarbonyl, C₂-C₇ alkenyl, or C₁-C₇ alkyl optionally substituted with at least one substituent (e.g. one, two, three or four substituents independently) selected from carboxyl, hydroxyl, amino, C₁-C₆, or C₁-C₄, alkylamino, di-C₁-C₆, or C₁-C₄, alkylamino, NH(CH₂)₂OH, C₁-C₆, or C₁-C₄, alkoxy, C₁-C₆, or C₁-C₄, alkylthio, C₁-C₆, or C₁-C₄, alkoxycarbonyl, and a saturated or unsaturated 3- to 10-membered ring system which may comprise at least one ring heteroatom (e.g. one, two, three or four ring heteroatoms independently) selected from nitrogen, oxygen and sulphur, the ring system being optionally substituted by at least one substituent (e.g. one, two, three or four substituents independently) selected from halogen (e.g. fluorine, chlorine, bromine or iodine), hydroxyl, oxo, carboxyl, cyano, C₁-C₆, or C₁-C₄, alkyl, C₁-C₆, or C₁-C₄, hydroxyalkyl, —NR⁸R⁹, —(CH₂)ᵣNR¹⁰R¹¹ and —CONR¹²R¹³.

In an aspect of the invention, R⁶ and R⁷ each independently represent hydrogen, pyrrolidinyl, C₁-C₂ alkylcarbonyl, C₅-C₇ alkenyl, or C₁-C₇ alkyl optionally substituted with one or two substituents independently selected from carboxyl, hydroxyl, amino, C₁-C₂ alkylamino, di-C₁-C₂ alkylamino, —NH(CH₂)₂OH, C₁-C₂ alkoxy, C₁-C₂ alkylthio, C₁-C₂ alkoxycarbonyl, and a saturated or unsaturated 3- to 10-membered ring system which may comprise at least one ring heteroatom (e.g. one, two, three or four ring heteroatoms independently) selected from nitrogen, oxygen and sulphur, the ring system being optionally substituted by at least one substituent (e.g. one, two, three or four substituents independently) selected from fluorine, hydroxyl, oxo, carboxyl, cyano, C₁-C₂ alkyl, C₁-C₂ hydroxyalkyl, —NR⁸R⁹, —(CH₂)ᵣNR¹⁰R¹¹ and —CONR¹²R¹³.

In a further aspect, R⁶ and R⁷ each independently represent hydrogen, pyrrolidinyl, methylcarbonyl, C₇ alkenyl, or C₁-C₇ alkyl optionally substituted with one or two substituents independently selected from carboxyl, hydroxyl, methylamino, di-methylamino, —NH(CH₂)₂OH, methylthio, C₁-C₂ alkoxycarbonyl, and a saturated or unsaturated 3- to 10-membered ring system which may comprise one, two or three ring heteroatoms independently selected from nitrogen, oxygen and sulphur, the ring system being optionally substituted by one or two substituents independently selected from fluorine, hydroxyl, oxo, C₁-C₂ alkyl and hydroxymethyl.

The saturated or unsaturated 3- to 10-membered ring system defined above may be monocyclic or polycyclic (e.g. bicyclic) and may have alicyclic or aromatic properties. An unsaturated ring system will be partially or fully unsaturated. Examples of ring systems that may be used include cyclopropyl, cyclobutyl, cyclopentyl, cyclohexyl, cycloheptyl, cyclopentenyl, cyclohexenyl, bicyclo[2.2.1]hept-2-yl, bicyclo[2.2.1]hept-5-en-2-yl, phenyl, 3,4-dihydro-2H-pyranyl, pyrrolidinyl, piperidinyl, piperazinyl, phenyl, pyrazolyl, thiazolidinyl, indanyl, thienyl, isoxazolyl, thiadiazolyl, pyrrolyl, furyl, thiazolyl, indolyl, imidazolyl, benzimidazolyl, triazolyl, tetrazolyl and pyridinyl.

In one aspect, the saturated or unsaturated 3- to 10-membered ring system is selected from cyclopropyl, cyclohexenyl, phenyl, thienyl, pyridinyl, furyl, bicyclo[2.2.1]hept-5-en-2-yl, 3,4-dihydro-2H-pyranyl, thiazolyl, pyrrolyl, pyrazolyl, imidazolyl and thiadiazolyl.

In another embodiment, R⁶ and R⁷ together with the nitrogen atom to which they are attached may form a saturated six-membered heterocyclic Ting which may comprise a second ring heteroatom selected from nitrogen and oxygen, the ring being optionally substituted by at least one substituent (e.g. one, two, three or four substituents independently) selected from hydroxyl, halogen (e.g. fluorine, chlorine, bromine or iodine), C₁-C₆, or C₁-C₄, alkyl and C₁-C₆, or C₁-C₄, hydroxyalkyl. Examples of heterocyclic rings that may be formed include piperidinyl, piperazinyl and morpholinyl.

In one aspect, R⁶ and R⁷ together with the nitrogen atom to which they are attached may form a saturated six-membered heterocyclic ring which may comprise a second ring heteroatom selected from nitrogen and oxygen, the ring being optionally substituted by one or two substituents independently selected from C₁-C₂ alkyl and C₁-C₂ hydroxyalkyl.

R⁸ and R⁹ each independently represent a hydrogen atom or a C₁-C₆, or C₁-C₄, alkyl, C₂-C₆, or C₂-C₄, hydroxyalkyl or C₃-C₈, or C₅-C₆, cycloalkyl group, or R⁸ and R⁹ together with the nitrogen atom to which they are attached form a 3- to 8-membered saturated heterocyclic ring (e.g. pyrrolidinyl or piperidinyl).

R¹⁰ and R¹¹ each independently represent a hydrogen atom or a C₁-C₆, or C₁-C₄, alkyl, C₂-C₆, or C₂-C₄, hydroxyalkyl or C₃-C₈, or C₅-C₆, cycloalkyl group, or R¹² and R¹³ together with the nitrogen atom to which they are attached form a 3- to 8-membered saturated heterocyclic ring (e.g. pyrrolidinyl or piperidinyl).

R¹² and R¹³ each independently represent a hydrogen atom or a C₁-C₆, or C₁-C₄, alkyl, C₂-C₆, or C₂-C₄, hydroxyalkyl or C₃-C₈, or C₅-C₆, cycloalkyl group, or R¹² and R¹³ together with the nitrogen atom to which they are attached form a 3- to 8-membered saturated heterocyclic ring (e.g. pyrrolidinyl or piperidinyl).

Examples of compounds of the invention include
2-(1-Adamantyl)-N-(4-methylquinolin-5-yl)acetamide,
2-(1-Adamantyl)-N-(2-chloroquinolin-5-yl)acetamide,
2-(1-Adamantyl)-N-(6-methylquinolin-5-yl)acetamide,
2-(1-Adamantyl)-N-(2-chloro-6-methylquinolin-5-yl)acetamide,
2-(1-Adamantyl)-N-(6-chloroquinolin-5-yl)acetamide,
2-(1-Adamantyl)-N-{2-[(3-hydroxypropyl)amino]quinolin-5-yl}acetamide,
2-(1-Adamantyl)-N-(2-{[(2R)-2-hydroxypropyl]amino}quinolin-5-yl)acetamide,
2-(1-Adamantyl)-N-(2-{[(2S)-2-hydroxypropyl]amino}quinolin-5-yl)acetamide,
2-(1-Adamantyl)-N-{2-[(2-hydroxyethyl)amino]quinolin-5-yl}acetamide,
N-(1-Adamantyl)-N-(2-{[3-(4-methylpiperazin-1-yl)propyl]amino}quinolin-5-yl)acetamide,
2-(1-Adamantyl)-N-(2-{[(2S)-2,3-dihydroxypropyl]amino}quinolin-5-yl)acetamide,
2-(1-Adamantyl)-N-{2-[(3-hydroxypropyl)amino]-6-methylquinolin-5-yl}acetamide,
2-(1-Adamantyl)-N-{2-[(2-hydroxyethyl)amino]-6-methylquinolin-5-yl}acetamide,
2-(1-Adamantyl)-N-{2-[[2-(dimethylamino)ethyl](methyl)amino]-6-methylquinolin-5-yl}acetamide,
2-(1-Adamantyl)-N-{2-[(2-aminoethyl)amino]quinolin-5-yl}acetamide,
2-(1-Adamantyl)-N-{2-[(3-aminopropyl)amino]quinolin-5-yl}acetamide trifluoroacetate,
2-(1-Adamantyl)-N-[2-({2-[(2-hydroxyethyl)amino]ethyl}amino)quinolin-5-yl]acetamide dihydrochloride,
2-(1-Adamantyl)-N-{2-[(2-aminoethyl)(2-hydroxyethyl)amino]quinolin-5-yl}acetamide,
2-(1-Adamantyl)-N-[2-({2-[(cyclohex-3-en-1-ylmethyl)amino]ethyl}amino)quinolin-5-yl]acetamide,
2-(1-Adamantyl)-N-(2-{[2-(isobutylamino)ethyl]amino}quinolin-5-yl)acetamide,
2-(1-Adamantyl)-N-[2-({2-[(4-methylbenzyl)amino]ethyl}amino)quinolin-5-yl]acetamide,
{[2-({5-[(1-Adamantylacetyl)amino]quinolin-2-yl}amino)ethyl]amino}acetic acid,
2-(1-Adamantyl)-N-(2-{[2-(benzylamino)ethyl]amino}quinolin-5-yl)acetamide,
2-(1-Adamantyl)-N-(2-{[2-(hexylamino)ethyl]amino}quinolin-5-yl)acetamide,
2-(1-Adamantyl)-N-(2-{[2-(propylamino)ethyl]amino}quinolin-5-yl)acetamide,
2-(1-Adamantyl)-N-(2-{[2-(heptylamino)ethyl]amino}quinolin-5-yl)acetamide,
2-(1-Adamantyl)-N-[2-({2-[(thien-2-ylmethyl)amino]ethyl}amino)quinolin-5-yl]acetamide,
2-(1-Adamantyl)-N-[2-({2-[(pyridin-2-ylmethyl)amino]ethyl}amino)quinolin-5-yl]acetamide,
2-(1-Adamantyl)-N-[2-({2-[(3-hydroxybenzyl)amino]ethyl}amino)quinolin-5-yl]acetamide,
2-(1-Adamantyl)-N-{2-[(2-{[(5-methyl-2-furyl)methyl]amino}ethyl)amino]quinolin-5-yl}acetamide,
2-(1-Adamantyl)-N-{2-[(2-{[(3-methylthien-2-yl)methyl]amino}ethyl)-amino]quinolin-5-yl}acetamide,
2-(1-Adamantyl)-N-[2-({2-[(thien-3-ylmethyl)amino]ethyl}amino)quinolin-5-yl]acetamide,
2-(1-Adamantyl)-N-(2-{[2-(pentylamino)ethyl]amino}quinolin-5-yl)acetamide,
2-(1-Adamantyl)-N-(2-{[2-(isopentylamino)ethyl]amino}quinolin-5-yl)acetamide,
2-(1-Adamantyl)-N-(2-{[2-(butylamino)ethyl]amino}quinolin-5-yl)acetamide,
2-(1-Adamantyl)-N-[2-({2-[(3,3-dimethylbutyl)amino]ethyl}amino)quinolin-5-yl]acetamide,
2-(1-Adamantyl)-N-[2-({2-[(bicyclo[2.2.1]hept-5-en-2-ylmethyl)amino]-ethyl}amino)quinolin-5-yl]acetamide,
2-(1-Adamantyl)-N-[2-({2-[(3-methylbenzyl)amino]ethyl}amino)quinolin-5-yl]acetamide,
2-(1-Adamantyl)-N-[2-({2-[(2-furylmethyl)amino]ethyl}amino)quinolin-5-yl]acetamide,
2-(1-Adamantyl)-N-[2-({2-[(4-fluorobenzyl)amino]ethyl}amino)quinolin-5-yl]acetamide,
2-(1-Adamantyl)-N-[2-({2-[(3-fluorobenzyl)amino]ethyl}amino)quinolin-5-yl]acetamide,
2-(1-Adamantyl)-N-[2-({2-[(3-furylmethyl)amino]ethyl}amino)quinolin-5-yl]acetamide,
2-(1-Adamantyl)-N-[2-({2-[(2-hydroxybenzyl)amino]ethyl}amino)quinolin-5-yl]acetamide,
2-(1-Adamantyl)-N-[2-({2-[(2E)-hex-2-enylamino]ethyl}amino)quinolin-5-yl]acetamide,
2-(1-Adamantyl)-N-[2-({2-[(2-fluorobenzyl)amino]ethyl}amino)quinolin-5-yl]acetamide,
2-(1-Adamantyl)-N-[2-({2-[(cyclopropylmethyl)amino]ethyl}amino)quinolin-5-yl]acetamide,
2-(1-Adamantyl)-N-[2-({2-[(5-hydroxypentyl)amino]ethyl}amino)quinolin-5-yl]acetamide,
2-(1-Adamantyl)-N-{2-[(2-{[(6-methylpyridin-2-yl)methyl]amino}ethyl)-amino]quinolin-5-yl}acetamide,
2-(1-Adamantyl)-N-[2-({2-[(2-methylbenzyl)amino]ethyl}amino)quinolin-5-yl]acetamide,
2-(1-Adamantyl)-N-[2-({2-[(2-phenylethyl)amino]ethyl}amino)quinolin-5-yl]acetamide,
2-(1-Adamantyl)-N-{2-[(2-{[(5-methylthien-2-yl)methyl]amino}ethyl)-amino]quinolin-5-yl}acetamide,
2-(1-Adamantyl)-N-(2-{[2-({[5-(hydroxymethyl)-2-furyl]methyl}amino)-ethyl]amino}quinolin-5-yl)acetamide,
2-(1-Adamantyl)-N-{2-[(2-{[3-(methylthio)propyl]amino}ethyl)amino]quinolin-5-yl}acetamide,
2-(1-Adamantyl)-N-[2-({2-[(3,4-dihydro-2H-pyran-5-ylmethyl)amino]-ethyl}amino)quinolin-5-yl]acetamide,
2-(1-Adamantyl)-N-[2-({2-[(1,3-thiazol-2-ylmethyl)amino]ethyl}amino)quinolin-5-yl]acetamide,
2-(1-Adamantyl)-N-[2-({2-[(3-hydroxy-2,2-dimethylpropyl)amino]-ethyl}amino)quinolin-5-yl]acetamide,
2-(1-Adamantyl)-N-{2-[(2-{[3-(methylthio)butyl]amino}ethyl)amino]quinolin-5-yl}acetamide,
2-(1-Adamantyl)-N-[2-({2-[(2-ethylbutyl)amino]ethyl}amino)quinolin-5-yl]acetamide,
2-(1-Adamantyl)-N-{2-[(2-{[(2E)-2-methylbut-2-enyl]amino}ethyl)amino]quinolin-5-yl}acetamide,
2-(1-Adamantyl)-N-{2-[(2-{[(2E)-2-methylpent-2-enyl]amino}ethyl)amino]quinolin-5-yl}acetamide,
2-(1-Adamantyl)-N-{2-[(2-{[(1-methyl-1H-pyrrol-2-yl)methyl]amino}ethyl)amino]-quinolin-5-yl}acetamide,
2-(1-Adamantyl)-N-{2-[(2-{[(1-oxidopyridin-4-yl)methyl]amino}ethyl)-amino]quinolin-5-yl}acetamide,
2-(1-Adamantyl)-N-[2-({2-[(2-ethyl-3-methylbutyl)amino]ethyl}amino)quinolin-5-yl]acetamide,
2-(1-Adamantyl)-N-[2-({2-[(1H-pyrazol-3-ylmethyl)amino]ethyl}amino)quinolin-5-yl]acetamide,
Ethyl {[2-({5-[(1-adamantylacetyl)amino]quinolin-2-yl}amino)ethyl]amino}acetate,
2-(1-Adamantyl)-N-[2-({2-[(2,2-dimethylpent-4-enyl)amino]ethyl}amino)quinolin-5-yl]acetamide,
2-(1-Adamantyl)-N-{2-[(2-{[(1-methyl-1H-imidazol-2-yl)methyl]amino}ethyl)-amino]quinolin-5-yl}acetamide,
2-(1-Adamantyl)-N-{2-[(2-{[(2-ethyl-1H-imidazol-5-yl)methyl]amino}ethyl)amino]-quinolin-5-yl}acetamide,
2-(1-Adamantyl)-N-[2-({2-[(1,2,3-thiadiazol-4-ylmethyl)amino]ethyl}amino)quinolin-5-yl]acetamide,
2-(1-Adamantyl)-N-[2-({3-[(cyclohex-3-en-1-ylmethyl)amino]propyl}amino)quinolin-5-yl]acetamide,
2-(1-Adamantyl)-N-(2-{[3-(isobutylamino)propyl]amino}quinolin-5-yl)acetamide,
2-(1-Adamantyl)-N-[2-({3-[(4-methylbenzyl)amino]propyl}amino)quinolin-5-yl]acetamide,
{[3-({5-[(1-Adamantylacetyl)amino]quinolin-2-yl}amino)propyl]amino}acetic acid,
2-(1-Adamantyl)-N-(2-{[3-(benzylamino)propyl]amino}quinolin-5-yl)acetamide,
2-(1-Adamantyl)-N-(2-{[3-(hexylamino)propyl]amino}quinolin-5-yl)acetamide,
2-(1-Adamantyl)-N-(2-{[3-(propylamino)propyl]amino}quinolin-5-yl)acetamide,
2-(1-Adamantyl)-N-(2-{[3-(heptylamino)propyl]amino}quinolin-5-yl)acetamide,
2-(1-Adamantyl)-N-[2-({3-[(thien-2-ylmethyl)amino]propyl}amino)quinolin-5-yl]acetamide,
2-(1-Adamantyl)-N-[2-({3-[(pyridin-2-ylmethyl)amino]propyl}amino)quinolin-5-yl]acetamide,
2-(1-Adamantyl)-N-[2-({3-[(3-hydroxybenzyl)amino]propyl}amino)quinolin-5-yl]acetamide,
2-(1-Adamantyl)-N-{2-[(3-{[(5-methyl-2-furyl)methyl]amino}propyl)amino]quinolin-5-yl}acetamide,
2-(1-Adamantyl)-N-{2-[(3-{[(3-methylthien-2-yl)methyl]amino}propyl)amino]-quinolin-5-yl}acetamide,
2-(1-Adamantyl)-N-[2-({3-[(thien-3-ylmethyl)amino]propyl}amino)quinolin-5-yl]acetamide,
2-(1-Adamantyl)-N-(2-{[3-(pentylamino)propyl]amino}quinolin-5-yl)acetamide,
2-(1-Adamantyl)-N-(2-{[3-(isopentylamino)propyl]amino}quinolin-5-yl)acetamide,
2-(1-Adamantyl)-N-(2-{[3-(butylamino)propyl]amino}quinolin-5-yl)acetamide,
2-(1-Adamantyl)-N-[2-({3-[(3,3-dimethylbutyl)amino]propyl}amino)quinolin-5-yl]acetamide,
2-(1-Adamantyl)-N-[2-({3-[(bicyclo[2.2.1]hept-5-en-2-ylmethyl)amino]propyl}-amino)quinolin-5-yl]acetamide,
2-(1-Adamantyl)-N-[2-({3-[(3-methylbenzyl)amino]propyl}amino)quinolin-5-yl]acetamide,
2-(1-Adamantyl)-N-[2-({3-[(2-furylmethyl)amino]propyl}amino)quinolin-5-yl]acetamide,
2-(1-Adamantyl)-N-[2-({3-[(4-fluorobenzyl)amino]propyl}amino)quinolin-5-yl]acetamide,
2-(1-Adamantyl)-N-[2-({3-[(3-fluorobenzyl)amino]propyl}amino)quinolin-5-yl]acetamide,
2-(1-Adamantyl)-N-[2-({3-[(3-furylmethyl)amino]propyl}amino)quinolin-5-yl]acetamide,
2-(1-Adamantyl)-N-[2-({3-[(2-hydroxybenzyl)amino]propyl}amino)quinolin-5-yl]acetamide,
2-(1-Adamantyl)-N-[2-({3-[(2E)-hex-2-enylamino]propyl}aminoquinolin-5-yl]acetamide,
2-(1-Adamantyl)-N-[2-({3-[(2-fluorobenzyl)amino]propyl}amino)quinolin-5-yl]acetamide,
2-(1-Adamantyl)-N-[2-({3-[(cyclopropylmethyl)amino]propyl}amino)quinolin-5-yl]acetamide,
2-(1-Adamantyl)-N-[2-({3-[(1H-imidazol-2-ylmethyl)amino]propyl}amino)quinolin-5-yl]acetamide,
2-(1-Adamantyl)-N-[2-({3-[(5-hydroxypentyl)amino]propyl}amino)quinolin-5-yl]acetamide,
2-(1-Adamantyl)-N-{2-[(3-{[(6-methylpyridin-2-yl)methyl]amino}propyl)amino]-quinolin-5-yl}acetamide,
2-(1-Adamantyl)-N-[2-({3-[(2-methylbenzyl)amino]propyl}amino)quinolin-5-yl]acetamide,
2-(1-Adamantyl)-N-[2-({3-[(2-phenylethyl)amino]propyl}amino)quinolin-5-yl]acetamide,
2-(1-Adamantyl)-N-{2-[(3-{[(5-ethyl-2-furyl)methyl]amino}propyl)amino]quinolin-5-yl}acetamide,
2-(1-Adamantyl)-N-{2-[(3-{[(5-methylthien-2-yl)methyl]amino}propyl)amino]-quinolin-5-yl}acetamide,
2-(1-Adamantyl)-N-{2-[(3-{[3-(methylthio)propyl]amino}propyl)amino]quinolin-5-yl}acetamide,
2-(1-Adamantyl)-N-[2-({3-[(3,4-dihydro-2H-pyran-5-ylmethyl)amino]propyl}amino)-quinolin-5-yl]acetamide,
2-(1-Adamantyl)-N-[2-({3-[(1,3-thiazol-2-ylmethyl)amino]propyl}amino)quinolin-5-yl]acetamide,
2-(1-Adamantyl)-N-[2-({3-[(3-hydroxy-2,2-dimethylpropyl)amino]propyl}amino)-quinolin-5-yl]acetamide,
2-(1-Adamantyl)-N-{2-[(3-{[3-(methylthio)butyl]amino}propyl)amino]quinolin-5-yl}acetamide,
2-(1-Adamantyl)-N-{2-[(3-{[3-(dimethylamino)-2,2-dimethylpropyl]-amino}propyl)amino]quinolin-5-yl}acetamide,
2-(1-Adamantyl)-N-[2-({3-[(2-ethylbutyl)amino]propyl}amino)quinolin-5-yl]acetamide,
2-(1-Adamantyl)-N-{2-[(3-{[(2E)-2-methylbut-2-enyl]amino}propyl)amino]quinolin-5-yl}acetamide,
2-(1-Adamantyl)-N-{2-[(3-{[(2E)-2-methylpent-2-enyl]amino}propyl)-amino]quinolin-5-yl}acetamide,
2-(1-Adamantyl)-N-{2-[(3-{[(1-methyl-1H-pyrrol-2-yl)methyl]amino}propyl)amino]-quinolin-5-yl}acetamide,
2-(1-Adamantyl)-N-[2-({3-[(2-ethyl-3-methylbutyl)amino]propyl}amino)quinolin-5-yl]acetamide,
Ethyl {[3-({5-[(1-adamantylacetyl)amino]quinolin-2-yl}amino)propyl]amino}acetate,
2-(1-Adamantyl)-N-[2-({3-[(2,2-dimethylpent-4-enyl)amino]propyl}amino)quinolin-5-yl]acetamide,
2-(1-Adamantyl)-N-[2-({3-[(1,2,3-thiadiazol-4-ylmethyl)amino]propyl}amino)-quinolin-5-yl]acetamide,
2-(1-Adamantyl)-N-{2-[(4-hydroxybutyl)amino]quinolin-5-yl}acetamide,
Methyl 3-({5-[(1-adamantylacetyl)amino]quinolin-2-yl}amino)propanoate,
N-(2-{[2-(Acetylamino)ethyl]amino}quinolin-5-yl)-2-(1-adamantyl)acetamide,
2-(1-Adamantyl)-N-{2-[(1-benzyl-2-hydroxyethyl)amino]quinolin-5-yl}acetamide,
2-(1-Adamantyl)-N-(2-{[1-(hydroxymethyl)propyl]amino}quinolin-5-yl)acetamide,
2-(1-Adamantyl)-N-(2-{[(2S)-2-hydroxycyclohexyl]amino}quinolin-5-yl)acetamide,
2-(1-Adamantyl)-N-{2-[(2-morpholin-4-ylethyl)amino]quinolin-5-yl}acetamide,
2-(1-Adamantyl)-N-{2-[(2-hydroxy-2-phenylethyl)amino]quinolin-5-yl}acetamide,
2-(1-Adamantyl)-N-{2-[(2-hydroxy-1-methylethyl)amino]quinolin-5-yl}acetamide,
2-(1-Adamantyl)-N-{2-[(2-methoxyethyl)amino]quinolin-5-yl}acetamide,
2-(1-Adamantyl)-N-(2-{[2-(5-methoxy-1H-indol-3-yl)ethyl]amino}quinolin-5-yl)acetamide,
2-(1-Adamantyl)-N-(2-{[2-(4-hydroxyphenyl)ethyl]amino}quinolin-5-yl)acetamide,
2-(1-Adamantyl)-N-{2-[(2-hydroxy-1-phenylethyl)amino]quinolin-5-yl}acetamide,
2-(1-Adamantyl)-N-(2-{[1-(hydroxymethyl)-3-methylbutyl]amino}quinolin-5-yl)-acetamide,
2-(1-Adamantyl)-N-[2-(isobutylamino)quinolin-5-yl]acetamide,
2-(1-Adamantyl)-N-(2-{[1-(hydroxymethyl)propyl]amino}quinolin-5-yl)acetamide,
2-(1-Adamantyl)-N-{2-[(3-ethoxypropyl)amino]quinolin-5-yl}acetamide,
2-(1-Adamantyl)-N-{2-[(2-hydroxy-2,3-dihydro-1H-inden-1-yl)amino]quinolin-5-yl}acetamide,
2-(1-Adamantyl)-N-(2-{[2-(2-hydroxyethoxy)ethyl]amino}quinolin-5-yl)acetamide,
2-(1-Adamantyl)-N-[2-(cyclobutylamino)quinolin-5-yl]acetamide,
2-(1-Adamantyl)-N-(2-{[3-(2-oxopyrrolidin-1-yl)propyl]amino}quinolin-5-yl)-acetamide,
2-(1-Adamantyl)-N-{2-[(1-benzylpyrrolidin-3-yl)amino]quinolin-5-yl}acetamide,
2-(1-Adamantyl)-N-(2-{[2-(methylthio)ethyl]amino}quinolin-5-yl)acetamide,
2-(1-Adamantyl)-N-{2-[(3-methoxypropyl)amino]quinolin-5-yl}acetamide,
2-(1-Adamantyl)-N-{2-[(2-phenoxyethyl)amino]quinolin-5-yl}acetamide,
2-(1-Adamantyl)-N-(2-{[2-(1,3-benzodioxol-5-yl)ethyl]amino}quinolin-5-yl)-acetamide,
2-(1-Adamantyl)-N-(2-{[2-(4-phenoxyphenyl)ethyl]amino}quinolin-5-yl)acetamide,
2-(1-Adamantyl)-N-(2-{[2-(1H-indol-3-yl)ethyl]amino}quinolin-5-yl)acetamide,
2-(1-Adamantyl)-N-{2-[(2-piperidin-1-ylethyl)amino]quinolin-5-yl}acetamide,
2-(1-Adamantyl)-N-(2-{[2-hydroxy-1-(hydroxymethyl)ethyl]amino}quinolin-5-yl)acetamide,
2-(1-Adamantyl)-N-(2-{[(1R)-1-(hydroxymethyl)-2,2-dimethylpropyl]-amino}quinolin-5-yl)acetamide,
2-(1-Adamantyl)-N-(2-{[2-(3-hydroxyphenyl)ethyl]amino}quinolin-5-yl)acetamide,
2-(1-Adamantyl)-N-(2-{[(1S,3R,4R)-3-(hydroxymethyl)bicyclo[2.2.1]hept-2-yl]amino}quinolin-5-yl)acetamide,
2-(1-Adamantyl)-N-(2-{[(1R,3R,4S)-3-(hydroxymethyl)bicyclo[2.2.1]hept-2-yl]amino}quinolin-5-yl)acetamide,
2-(1-Adamantyl)-N-(2-{[2-(benzyloxy)-1-(hydroxymethyl)ethyl]amino}quinolin-5-yl)acetamide,
2-(1-Adamantyl)-N-{2-[(cyclopropylmethyl)amino]quinolin-5-yl}acetamide,
2-(1-Adamantyl)-N-(2-{[2-(4-chlorophenyl)-1-methylethyl]amino}quinolin-5-yl)-acetamide,
2-(1-Adamantyl)-N-(2-{[1-(hydroxymethyl)propyl]amino}quinolin-5-yl)acetamide,
2-(1-Adamantyl)-N-{2-[(2-{4-[(methylsulfonyl)amino]phenyl}ethyl)amino]quinolin-5-yl}acetamide,
2-(1-Adamantyl)-N-[2-({2-[bis(2-hydroxyethyl)amino]ethyl}amino)quinolin-5-yl]acetamide,
2-(1-Adamantyl)-N-quinolin-5-ylacetamide,
2-(1-Adamantyl)-N-isoquinolin-5-ylacetamide,
2-(1-Adamantyl)-N-[2-(3-{[(1R)-2-hydroxy-1-methylethyl]amino}propyl)quinolin-5-yl]acetamide dihydrochloride,
2-(1-Adamantyl)-N-(2-{2-[benzyl(2-hydroxyethyl)amino]ethoxy}quinolin-5-yl)acetamide,
2-(1-Adamantyl)-N-(2-{2-[(2-hydroxyethyl)amino]ethoxy}quinolin-5-yl)acetamide,
2-(1-Adamantyl)-N-{2-[bis(2-hydroxyethyl)amino]quinolin-5-yl}acetamide,
2-(1-Adamantyl)-N-[8-({2-[(2-hydroxyethyl)amino]ethyl}amino)quinolin-5-yl]acetamide trihydrochloride,
2-(1-Adamantyl)-N-{8-[(2-aminoethyl)thio]quinolin-5-yl}acetamide,
N-(1-Adamantylmethyl)-6-chloro-2-[3-(methylamino)propyl]quinoline-5-carboxamide sesquihydrochloride dihydrate,
N-(1-Adamantylmethyl)-2-{3-[(3-hydroxypropyl)amino]propyl}quinoline-4-carboxamide benzoic acid salt,
N-(1-Adamantylmethyl)-8-[3-(methylamino)propyl]quinoline-4-carboxamide dihydrochloride,
N-(1-Adamantylmethyl)-6-chloro-2-(piperazin-1-ylmethyl)quinoline-5-carboxamide hydrochloride,
N-(1-Adamantylmethyl)-quinoline-5-carboxamide trifluoroacetate,
N-(1-Adamantylmethyl)-2-{3-[(3-hydroxypropyl)amino]propyl}quinoline-5-carboxamide dihydrochloride,
N-(1-adamantylmethyl)-2-[3-(ethylamino)propyl]quinoline-5-carboxamide dihydrochloride,
2-(1-Adamantyl)-N-[2-({2-[(2-hydroxyethyl)amino]ethyl}amino)-6-methylquinolin-5-yl]acetamide hydrochloride,
2-(1-Adamantyl)-N-[2-({2-[(2-hydroxyethyl)amino]ethyl}amino)-6-chloroquinolin-5-yl]acetamide dihydrochloride,
2-(1-Adamantyl)-N-[2-({2-[(2-hydroxyethyl)amino]ethyl}amino)quinolin-5-yl]acetamide dihydrochloride,
2-(1-Adamantyl)-N-(2-{3-[(3-hydroxypropyl)amino]propyl}quinolin-5-yl)acetamide dihydrochloride,
2-(1-Adamantyl)-N-(6-methyl-2-piperazin-1-ylquinolin-5-yl)acetamide dihydrochloride,
2-(1-Adamantyl)-N-{2-[4-(2-hydroxyethyl)piperazin-1-yl]-6-methylquinolin-5-yl}acetamide dihydrochloride,
2-(1-Adamantyl)-N-[2-(4-aminopiperidin-1-yl)-6-methylquinolin-5-yl]acetamide dihydrochloride,
2-(1-Adamantyl)-N-(2-{4-[(2-hydroxyethyl)amino]piperidin-1-yl}-6-methylquinolin-5-yl)acetamide dihydrochloride,
2-(1-Adamantyl)-N-{2-[(35)-3-aminopyrrolidin-1-yl]-6-methylquinolin-5-yl}acetamide dihydrochloride,
(3S)—N-((3S)-1-{5-[(1-Adamantylacetyl)amino]-6-methylquinolin-2-yl}pyrrolidin-3-yl)-3-aminopyrrolidine-1-carboxamide dihydrochloride,
2-(1-Adamantyl)-N-{6-methyl-2-[(1-methylpiperidin-4-yl)amino]quinolin-5-yl}acetamide dihydrochloride,
2-(1-Adamantyl)-N-{6-methyl-2-[(3S)-3-(methylamino)pyrrolidin-1-yl]quinolin-5-yl}acetamide dihydrochloride,
2-(1-Adamantyl)-N-{2-[(3S)-3-(ethylamino)pyrrolidin-1-yl]-6-methylquinolin-5-yl}acetamide dihydrochloride,
2-(1-Adamantyl)-N-(2-{(3S)-3-[(2-hydroxyethyl)amino]pyrrolidin-1-yl}-6-methylquinolin-5-yl)acetamide dihydrochloride,
2-(1-Adamantyl)-N-(2-{(3S)-3-[(3-hydroxypropyl)amino]pyrrolidin-1-yl}-6-methylquinolin-5-yl)acetamide dihydrochloride,
2-(1-Adamantyl)-N-{6-chloro-2-[(3R)-3,4-dihydroxybutyl]quinolin-5-yl}acetamide hydrochloride,
2-(1-Adamantyl)-N-{6-chloro-2-[(3R)-3-hydroxy-4-(methylamino)butyl]quinolin-5-yl}acetamide dihydrochloride,
2-(1-Adamantyl)-N-{2-[(3R)-3-aminopyrrolidin-1-yl]-6-methylquinolin-5-yl}acetamide dihydrochloride,
2-(1-Adamantyl)-N-(6-chloro-2-{[2-(methylamino)ethyl]amino}quinolin-5-yl)acetamide dihydrochloride,
2-(1-Adamantyl)-N-(6-chloro-2-{methyl[3-(methylamino)propyl]amino}quinolin-5-yl)acetamide dihydrochloride,
2-(1-Adamantyl)-N-(6-chloro-2-{3-[(3-hydroxypropyl)amino]propyl}quinolin-5-yl)acetamide dihydrochloride,
2-(1-Adamantyl)-N-[6-chloro-2-({3-[(2-hydroxyethyl)amino]propyl}amino)quinolin-5-yl]acetamide dihydrochloride,
2-(1-Adamantyl)-N-(6-chloro-2-{[4-(2-hydroxyethyl)piperazin-1-yl]methyl}quinolin-5-yl)acetamide,
2-(1-Adamantyl)-N-[6-chloro-2-({[2-(methylamino)ethyl]amino}methyl)quinolin-5-yl]acetamide,
2-(1-Adamantyl)-N-{6-chloro-2-[({2-[(2-hydroxyethyl)amino]ethyl}amino)methyl]quinolin-5-yl}acetamide,
2-(1-Adamantyl)-N-[6-chloro-2-({[3-(methylamino)propyl]amino}methyl)quinolin-5-yl]acetamide bis(trifluoroacetate),
2-(1-Adamantyl)-N-(6-chloro-2-{[(3R)-pyrrolidin-3-ylamino]methyl}quinolin-5-yl)acetamide tris(trifluoroacetate),
2-(1-Adamantyl)-N-[2-({3-[(pyridin-2-ylmethyl)amino]propyl}amino)quinolin-5-yl]acetamide,
2-(1-Adamantyl)-N-[2-({2-[(2-hydroxyethyl)amino]propyl}amino)-6-methylquinolin-5-yl]acetamide hydrochloride,
N-(1-Adamantylmethyl)-2-[3-(methylamino)propyl]quinoline-5-carboxamide dihydrochloride,
2-(1-Adamantyl)-N-(6-methyl-2-{[3-(methylamino)propyl]amino}quinolin-5-yl)acetamide,
2-(1-Adamantyl)-N-(2-{2-[(3-hydroxypropyl)amino]ethyl}-6-methylquinolin-5-yl)acetamide dihydrochloride,
2-(1-Adamantyl)-N-[6-chloro-2-(piperazin-1-ylmethyl)quinolin-5-yl]acetamide trifluoroacetate,
2-(1-Adamantyl)-N-(6-chloro-2-piperazin-1-ylquinolin-5-yl)acetamide, and
N-(1-Adamantylmethyl)-6-chloro-2-{methyl[3-(methylamino)propyl]amino}quinoline-5-carboxamide.

The present invention further provides a process for the preparation of a compound of formula (I) as defined above, or a pharmaceutically acceptable salt or solvate thereof, which comprises:

(a) reacting a compound of formula

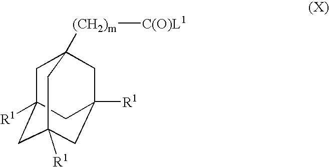

wherein L¹ represents a leaving group (e.g. hydroxyl or halogen) and m and R¹ are as defined in formula (I), with a compound of formula (XI), Ar—NH₂, wherein Ar is as defined in formula (I); or
(b) reacting a compound of formula

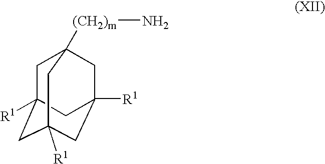

wherein m and R¹ are as defined in formula (I), with a compound of formula (XIII), Ar—C(O)-L², wherein L² represents a leaving group (e.g. hydroxyl or halogen) and Ar is as defined in formula (I); or
(c) when Ar represents a group

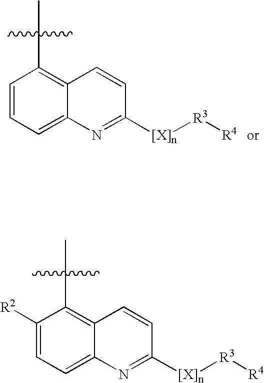

in which n is 1, X is >N—R⁵ and R² is other than a group of formula (III), reacting a compound of formula

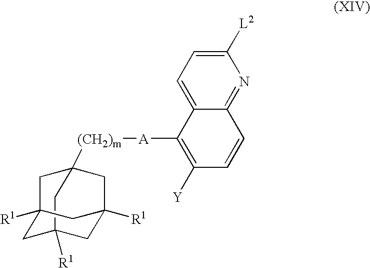

wherein L² is a leaving group (e.g. halogen, paratoluene sulphonate or methane sulphonate), Y is hydrogen or a group R^{2a} which represents halogen or C₁-C₆ alkyl optionally substituted by at least one substituent selected from hydroxyl, halogen and C₁-C₆ alkoxy, and m, A and R¹ are as defined in formula (I), with a compound of formula (XV), H—N(R⁵)-R³-R⁴, wherein R³, R⁴ and R⁵ are as defined in formula (I); or
(d) when Ar represents a group

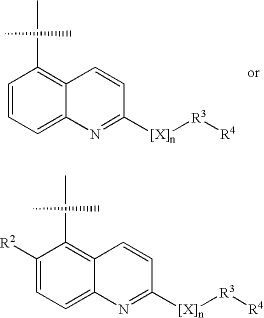

in which n is 0, R² is other than a group of formula (III) and R³ is an optionally substituted C₃-C₅ alkyl group, reacting a compound of formula (XIV) as defined in (c) above with a compound of formula

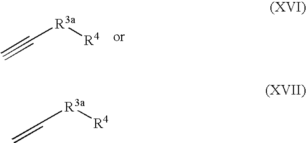

wherein R^{3a} represents a C₁-C₃ alkyl group optionally substituted as defined for R³ in formula (I) and R⁴ is as defined in formula (I), optionally followed by a hydrogenation reaction; or
(e) when Ar represents a group

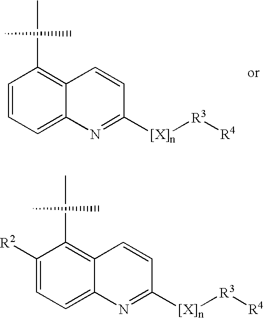

in which n is 0, R² is other than a group of formula (III), R³ is (CH₂)₂ and R⁴ is —NR⁶R⁷ reacting a compound of formula (XIV) as defined in (c) above with a compound of formula

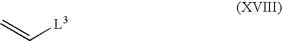

wherein L³ is a leaving group (eg. trialkyltin, dialkylboron or zinc), followed by reaction with a compound of formula (XIX), HNR⁶R⁷, wherein R⁶ and R⁷ are as defined in formula (I); or
(f) when Ar represents a group

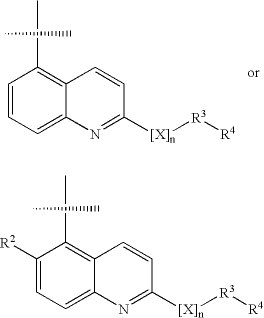

in which n is 0, R² is other than a group of formula (III), R³ is CH₂ and R⁴ is —NR⁶R⁷, reacting a compound of formula (XIV) as defined in (c) above with a compound of formula (XVIII) as defined in (e) above, followed by an oxidation reaction and then by reaction with a compound of formula (XIX) as defined in (e) above under reductive amination conditions;
and optionally after (a), (b), (c), (d), (e) or (f) carrying out one or more of the following:
converting the compound obtained to a further compound of the invention
forming a pharmaceutically acceptable salt or solvate of the compound.

In processes (a) and (b) the coupling reaction is conveniently carried out in an organic solvent such as dichloromethane, N,N-dimethylformamide or 1-methyl-2-pyrrolidinone. If L¹ or L² represent a hydroxyl group, it may be necessary or desirable to use a coupling agent such as bromo-tris-pyrrolidino-phosphonium hexafluorophosphate (PyBroP).

In process (c) the reaction may be performed in an organic solvent such as acetonitrile, N,N-dimethylformamide or 1-methyl-2-pyrrolidinone, and in the presence of a suitable base such as sodium hydride, triethylamine or potassium carbonate.

In process (d), if the compound of formula (XIV) is reacted with a compound of formula (XVI), then the reaction is conveniently carried out in an organic solvent such as acetonitrile, e.g. at ambient temperature (20° C.), in the presence of catalytic bistriphenylphosphine dichloride palladium(0), copper (I) iodide and a base (e.g. triethylamine). The subsequent hydrogenation reaction may use hydrogen gas with a catalyst such as 5% rhodium on carbon in a solvent, for example, ethyl acetate or ethanol, and at a pressure of 3 bar.

Alternatively, if the compound of formula (XIV) is reacted with a compound of formula (XVII), then it is preferred if the compound of formula (XVII) is pre-treated by reaction with a hydroborating reagent (e.g. 9-borabicyclo[3.3.1]nonane or catecholborane) in an organic solvent such as diethyl ether or tetrahydrofuran at a temperature in the range from, e.g. 0° C. to 80° C., in particular from 60° C. to 70° C., for about 2 to 3 hours. The pre-treated compound is then reacted with the compound of formula (XIV) in the presence of a suitable base (e.g. sodium hydroxide or tri-potassium orthophosphate) and a palladium catalyst (e.g. dichloro[1,1′-bis(diphenylphosphino)ferrocene]palladium (II) dichloromethane adduct), typically at a temperature in the range from 25° C. to 90° C., particularly from 60° C. to 70° C., for about 2 to 24 hours.

In process (e), the reaction with the vinyl compound of formula (XVIII) may conveniently be carried out in a solvent such as N,N-dimethylformamide and in the presence of catalytic dichlorobis(triphenylphosphine) palladium, at elevated temperature, e.g. at about 70° C. The subsequent addition reaction with the compound of formula (XIX) may be performed under acidic or basic conditions, for example, in acetic acid in a solvent such as methanol or isopropanol at elevated temperature, e.g. at about 100° C.

In process (f), the reaction of the vinyl compound of formula (XVIII) may be performed by procedures analogous to those outlined in the previous paragraph on process (e). The subsequent oxidation reaction may be carried out under standard conditions, for example, by using ozone followed by treatment with dimethylsulfide or triphenylphosphine in a suitable solvent such as dichloromethane, or, by using osmium tetroxide and sodium periodate in a suitable solvent such as 1,4-dioxane and water. The reductive amination step may be conveniently carried out in the presence of a reducing agent such as sodium cyanoborohydride, triacetoxyborohydride or sodium borohydride, in a polar solvent such as methanol, ethanol or dichloromethane either alone or in combination with acetic acid.

It will be appreciated that the processes (c), (d), (e) and (f) may be used to prepare other compounds of formula (I) comprising different isomeric forms of the group Ar, examples of which have previously been given.

Compounds of formulae (X), (XI), (XII), (XIII), (XIV), (XV), (XVI), (XVI), (XVIII) and (XIX) are either commercially available, are known in the literature or may be prepared using known techniques.

Compounds of formula (I) can be converted into further compounds of formula (I) using standard procedures. For example, compounds of formula (I) in which R² represents a halogen atom may be converted to a corresponding compound of formula (I) in which R² represents a C₁-C₆ alkyl group by reaction with an alkyl Grignard reagent (e.g. methyl magnesium bromide) in the presence of a catalyst such as [1,3-bis(diphenylphosphino)propane]dichloronickel (II) in a solvent such as tetrahydrofuran.

It will be appreciated by those skilled in the art that in the processes of the present invention certain functional groups such as hydroxyl or amino groups in the starting reagents or intermediate compounds may need to be protected by protecting groups. Thus, the preparation of the compounds of formula (I) may involve, at various stages, the addition and removal of one or more protecting groups.

The protection and deprotection of functional groups is described in ‘Protective Groups in Organic Chemistry’, edited by J. W. F. McOmie, Plenum Press (1973) and ‘Protective Groups in Organic Synthesis’, 2nd edition, T. W. Greene and P. G. M. Wuts, Wiley-Interscience (1991).

The compounds of formula (I) above may be converted to a pharmaceutically acceptable salt or solvate thereof, preferably an acid addition salt such as a hydrochloride, hydrobromide, phosphate, acetate, fumarate, maleate, tartrate, citrate, oxalate, methanesulphonate or p-toluenesulphonate, or an alkali metal salt such as a sodium or potassium salt.

Certain compounds of formula (I) are capable of existing in stereoisomeric forms. It will be understood that the invention encompasses all geometric and optical isomers of the compounds of formula (I) and mixtures thereof including racemates. Tautomers and mixtures thereof also form an aspect of the present invention.

The compounds of the present invention are advantageous in that they possess pharmacological activity. They are therefore indicated as pharmaceuticals for use in the treatment of rheumatoid arthritis, osteoarthritis, psoriasis, allergic dermatitis, asthma, chronic obstructive pulmonary disease (COPD), hyperresponsiveness of the airway, septic shock, glomerulonephritis, inflammatory bowel disease, Crohn's disease, ulcerative colitis, atherosclerosis, growth and metastases of malignant cells, myoblastic leukaemia, diabetes, Alzheimer's disease, meningitis, osteoporosis, burn injury, ischaemic heart disease, stroke, varicose veins, sarcoidosis, rhinitis, acute and chronic pain, multiple sclerosis, myeloma, bone loss associated with malignancy and inflammatory and neurodegenerative diseases of the eye such as seleritis, episcleritis, uveitis, Sjogrens syndrome-keratoconjuctivitis, sclerokeratitis, optic neuritis, diabetic retinopathy, retinitis pigmentosa, antimalarial-induced retinopathy.

Accordingly, the present invention provides a compound of formula (I), or a pharmaceutically acceptable salt or solvate thereof, as hereinbefore defined for use in therapy.

In another aspect, the invention provides the use of a compound of formula (I), or a pharmaceutically acceptable salt or solvate thereof, as hereinbefore defined in the manufacture of a medicament for use in therapy.

In the context of the present specification, the term “therapy” also includes “prophylaxis” unless there are specific indications to the contrary. The terms “therapeutic” and “therapeutically” should be construed accordingly.

The invention further provides a method of effecting immunosuppression (e.g. in the treatment of rheumatoid arthritis, osteoarthritis, irritable bowel disease, atherosclerosis or psoriasis) which comprises administering a therapeutically effective amount of a compound of formula (I), or a pharmaceutically acceptable salt or solvate thereof, as hereinbefore defined to a patient.

The invention also provides a method of treating an obstructive airways disease (e.g. asthma or COPD) which comprises administering to a patient a therapeutically effective amount of a compound of formula (I), or a pharmaceutically acceptable salt or solvate thereof, as hereinbefore defined to a patient.

For the above-mentioned therapeutic uses the dosage administered will, of course, vary with the compound employed, the mode of administration, the treatment desired and the disorder indicated. The daily dosage of the compound of formula (I)/salt/solvate (active ingredient) may be in the range from 0.001 mg/kg to 30 mg/kg.

The compounds of formula (I) and pharmaceutically acceptable salts and solvates thereof may be used on their own but will generally be administered in the form of a pharmaceutical composition in which the formula (I) compound/salt/solvate (active ingredient) is in association with a pharmaceutically acceptable adjuvant, diluent or carrier. Depending on the mode of administration, the pharmaceutical composition will preferably comprise from 0.05 to 99% w (percent by weight), more preferably from 0.10 to 70% w, of active ingredient, and, from 1 to 99.95% w, more preferably from 30 to 99.90% w, of a pharmaceutically acceptable adjuvant, diluent or carrier, all percentages by weight being based on total composition.

Thus, the present invention also provides a pharmaceutical composition comprising a compound of formula (I), or a pharmaceutically acceptable salt or solvate thereof, as hereinbefore defined in association with a pharmaceutically acceptable adjuvant, diluent or carrier.

The invention further provides a process for the preparation of a pharmaceutical composition of the invention which comprises mixing a compound of formula (I), or a pharmaceutically acceptable salt or solvate thereof, as hereinbefore defined with a pharmaceutically acceptable adjuvant, diluent or carrier.

The pharmaceutical composition of the invention may be administered topically (e.g. to the lung and/or airways or to the skin) in the form of solutions, suspensions, heptafluoroalkane aerosols and dry powder formulations; or systemically, e.g. by oral administration in the form of tablets, capsules, syrups, powders or granules, or by parenteral administration in the form of solutions or suspensions, or by subcutaneous administration or by rectal administration in the form of suppositories or transdermally.

The present invention will now be further explained by reference to the following illustrative examples.

### EXAMPLE 1

### 2-(1-Adamantyl)-N-(4-methylquinolin-5-yl)acetamide

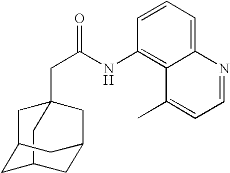

1-Adamantylacetic acid (233 mg) was dissolved in dichloromethane (15 mL) with dimethylformamide (50 μL). The solution was cooled to 0° C. and oxalyl chloride (158 μL) was slowly added. After 10 minutes stirring under nitrogen, the reaction was allowed to warm up slowly to room temperature and stirred for 2 hours under nitrogen. The solvent was evaporated under vacuum to give an oily residue that was triturated with toluene (15 mL) then concentrated down to an oil. This operation was repeated once. The residue was dissolved in dichloromethane (10 mL) and slowly added to a cold solution of 5-amino-4-methylquinoline (160 mg) in dichloromethane (5 mL) over an ice/water bath. The reaction was stirred under nitrogen for 10 minutes. A bright orange suspension developed. The reaction was treated by slow addition of triethylamine (420 μL) to give an ocre solution. This solution was stirred under nitrogen overnight, then partitioned with saturated aqueous sodium bicarbonate (30 mL). The dichloromethane phase was further washed with water. The organic phase was dried over magnesium sulphate, filtered and evaporated to give a yellow foam that was purified on silica gel using 2.5% methanol in dichloromethane. The column was washed with methanol and the collected fractions were evaporated then triturated with ether until a cream solid crystallized. This solid was filtered and dried to give 56 mg of the titled compound.

¹H NMR (400 MHz, DMSO-d₆) δ 9.78 (s, 1H); 8.72 (d, J=4.4 Hz, 1H); 7.94 (dd, J=8.5, 1.0 Hz, 1H); 7.72 (t, J=7.8 Hz, 1H); 7.38 (d, J=7.2 Hz, 1H); 7.32 (d, J=4.9 Hz, 1H); 2.80 (s, 3H); 2.14 (s, 2H); 1.97 (s, 3H); 1.67 (q, J=12.3 Hz, 6H); 1.72 (s, 6H).

MS: APCI(+ve) 335 (M+1).

### EXAMPLE 2

### 2-(1-Adamantyl)-N-(2-chloroquinolin-5-yl)acetamide

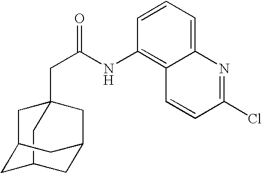

to 2-Chloroquinolin-5-amine (304 mg) in dichloromethane (5 mL) was slowly added to a solution of 1-adamantylacetyl chloride (330 mg) in dichloromethane (20 mL) that was prepared following the procedure described in Example 1. Work-up, isolation and purification were carried out following the procedure described in Example 1 to give 222 mg of solid.

¹H NMR (400 MHz, DMSO-d₆) δ 9.97 (s, 1H); 8.52 (d, J=9.1 Hz, 1H); 7.86-7.74 (m, 3H); 7.66 (d, J=9.0 Hz, 1H); 2.24 (s, 2H); 1.96 (s, 3H); 1.70 (s, 6H); 1.65 (q, J=12.9 Hz, 6H).

MS: APCI(+ve) 355/357(M+1).

### EXAMPLE 3

### 2-(1-Adamantyl)-N-(6-methylquinolin-5-yl)acetamide

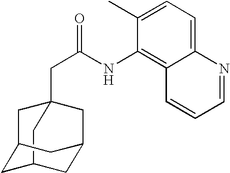

6-Methylquinolin-5-amine (0.120 g), 1-adamantylacetyl chloride (0.220 g) and triethylamine (0.35 mL) in dichloromethane (20 mL) were reacted together by the procedure given in Example 1 to afford the title compound as a white solid (0.071 g).

¹H NMR (400 MHz, CDCl₃) δ 9.01 (1H, s); 8.84 (1H, dd); 8.27 (1H, dt); 7.93 (1H, d); 7.60 (1H, d); 7.41 (1H, dd); 2.45 (3H, s); 2.32 (2H, s); 2.02 (3H, m); 1.84 (6H, m); 1.75 (6H, m).

MS: APCI(+ve) 335 (M+1)

MP: 204-295° C.

### EXAMPLE 4

### 2-(1-Adamantyl)-N-(2-chloro-6-methylquinolin-5-yl)acetamide

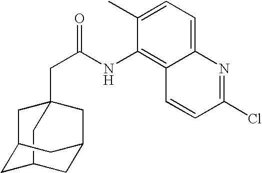

2-Chloro-6-methylquinolin-5-amine hydrochloride (0.450 g), 1-adamantylacetyl chloride (0.620 g) and triethylamine (1 mL) in dichloromethane (20 mL) were reacted together by the procedure given in Example 1 to afford the title compound as a white solid (0.60 g).

MS: APCI(+ve) 369 (M+1)

### EXAMPLE 5

### 2-(1-Adamantyl)-N-(6-chloroquinolin-5-yl)acetamide

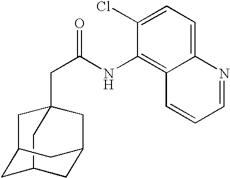

1-Adamantylacetyl chloride (0.120 g) in dichloromethane was added to a stirred suspension of 6-chloroquinolin-5-amine (0.089 g) and sodium hydride (60% dispersion in oil, 0.076 g) in dimethylformamide (2 mL). The mixture was stirred at room temperature for 24 hours, was poured into brine (5 mL) and was extracted into ethyl acetate (3×20 mL). The combined extracts were dried over anhydrous magnesium sulfate and concentrated to afford the titled compound as a white solid (0.039 g).

¹H NMR (400 MHz, DMSO-d₆) δ 9.98 (1H, s); 9.6 (1H, dd); 8.31 (1H, ddd); 8.05 (1H, d); 7.93 (1H, d); 7.70 (1H, dd); 2.32 (2H, s); 2.04 (3H, m); 1.84-1.59 (12H, m).

MS: APCI(+ve) 355/357 (M+1)

MP: 219-220° C.

### EXAMPLE 6

### 2-(1-Adamantyl)-N-{2-[(3-hydroxypropyl)amino]quinolin-5-yl}acetamide

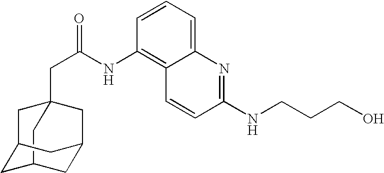

In a sealed tube, a solution of 2-(1-adamantyl)-N-{2-chloro-quinolin-5-yl}acetamide (Example 2) (75 mg), 3-aminopropan-1-ol (47 mg) in 1-methyl-2-pyrrolidinone (2 mL) was treated with potassium carbonate (29 mg) and the resulting suspension was heated to 130° C. for 36 hours. The solvent was then evaporated under vacuum and the dry residue was partitioned between dichloromethane (10 mL) and water (10 mL). The aqueous phase was further extracted with dichloromethane (10 mL) and the combined organic phases were washed with brine (15 mL), dried over magnesium sulfate, evaporated and the residue was purified by preparative reversed phase column chromatography (Xterra, acetonitrile/0.1% aqueous solution of ammonia at 7N in methanol) to give 15 mg of solid.

¹H NMR (400 MHz, DMSO-d₆) δ 9.60 (s, 1H); 7.95 (d, J=9.2 Hz, 1H); 7.40 (t, J=7.9 Hz, 1H); 7.28 (d, J=7.4 Hz, 2H); 7.02 (s, 1H); 6.76 (d, J=9.2 Hz, 1H); 4.68 (s, 1H); 3.50 (t, J=5.8 Hz, 2H); 3.43 (q, J=6.3 Hz, 2H); 2.17 (s, 2H); 1.96 (s, 3H); 1.76-1.60 (m, 14H).

MS: APCI(+ve) 394/395 (M+1).

### EXAMPLE 7

### 2-(1-Adamantyl)-N-(2-{[(2R)-2-hydroxypropyl]amino}quinolin-5-yl)acetamide

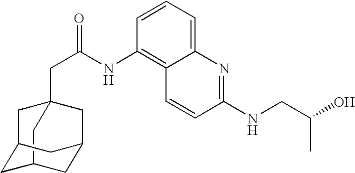

A solution of 2-(1-adamantyl)-N-{2-chloro-quinolin-5-yl}acetamide (Example 2) (75 mg) in 1-methyl-2-pyrrolidinone (2 mL) was treated with (2R)-1-aminopropan-2-ol (47 mg) and potassium carbonate (29 mg) following the procedure outlined in Example 6 to give 19 mg of a solid.

¹H NMR (400 MHz, DMSO-d₆) δ 9.59 (s, 1H); 7.94 (d, J=9.2 Hz, 1H); 7.38 (t, J=7.9 Hz, 1H); 7.27 (d, J=7.9 Hz, 2H); 6.99 (t, J=5.4 Hz, 1H); 6.82 (d, J=9.2 Hz, 1H); 4.98 (s, 1H); 3.84 (q, J=5.7 Hz, 1H); 3.41-3.27 (m, 2H); 2.16 (s, 2H); 1.94 (s, 3H); 1.67-1.59 (m, 12H); 1.10 (d, J=6.4 Hz, 3H).

MS: APCI(+ve) 394/395 (M+1).

### EXAMPLE 8

### 2-(1-Adamantyl)-N-(2-{[(2S)-2-hydroxypropyl]amino}quinolin-5-yl)acetamide

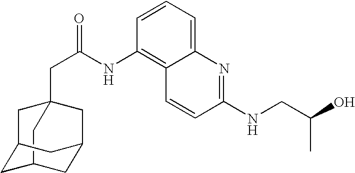

A solution of 2-(1-adamantyl)-N-{2-chloro-quinolin-5-yl}acetamide (Example 2) (75 mg) in 1-methyl-2-pyrrolidinone (2 mL) was treated with (2S)-1-aminopropan-2-ol (47 mg) and potassium carbonate (29 mg) following the procedure outlined in Example 6 to give 12 mg of a solid.

¹H NMR (400 MHz, DMSO-d₆) δ 9.59 (s, 1H); 7.94 (d, J=9.2 Hz, 1H); 7.38 (t, J=7.9 Hz, 1H); 7.27 (d, J=7.9 Hz, 2H); 6.99 (t, J=5.4 Hz, 1H); 6.82 (d, J=9.2 Hz, 1H); 4.98 (s, 1H); 3.84 (q, J=5.7 Hz, 1H); 3.41-3.27 (m, 2H); 2.16 (s, 2H); 1.94 (s, 3H); 1.67-1.59 (m, 12H); 1.10 (d, J=6.4 Hz, 3H).

MS: APCI(+ve) 394/395 (M+1).

### EXAMPLE 9

### 2-(1-Adamantyl)-N-{2-[(2-hydroxyethyl)amino]quinolin-5-yl}acetamide

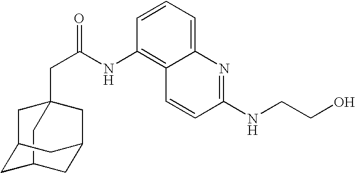

A solution of 2-(1-adamantyl)-N-{2-chloro-quinolin-5-yl}acetamide (Example 2) (75 mg) in 1-methyl-2-pyrrolidinone (2 mL) was treated with ethanolamine (38 mg) and potassium carbonate (29 mg) following the procedure outlined above in Example 6 to give 15 mg of a solid.

¹H NMR (400 MHz, DMSO-d₆) δ 9.60 (s, 1H); 7.95 (d, J=9.2 Hz, 1H); 7.40 (dd, J=9.0, 8.5 Hz, 1H); 7.29 (d, J=8.5 Hz, 2H); 7.03 (t, J=5.4 Hz, 1H); 6.80 (d, J=9.2 Hz, 1H); 3.59 (t, J=5.9 Hz, 2H); 3.47 (q, J=5.6 Hz, 2H); 2.17 (s, 2H); 1.96 (s, 3H); 1.72-1.57 (m, 12H).

MS: APCI(+ve) 380 (M+1).

### EXAMPLE 10

### N-(1-Adamantyl)-N-(2-{[3-(4-methylpiperazin-1-yl)propyl]amino}quinolin-5-yl)acetamide

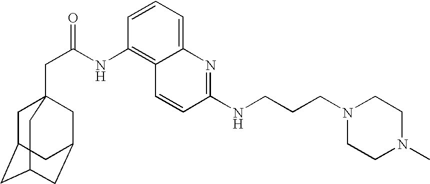

A solution of 2-(1-adamantyl)-N-{2-chloro-quinolin-5-yl}acetamide (Example 2) (75 mg) in 1-methyl-2-pyrrolidinone (2 mL) was treated with 3-(4-methylpiperazin-1-yl)propylamine (99 mg) and potassium carbonate (29 mg) following the procedure outlined in Example 6 to give 55 mg of a solid.

¹H NMR (400 MHz, DMSO-d₆) δ 9.59 (s, 1H); 7.94 (d, J=9.0 Hz, 1H); 7.39 (t, J=7.8 Hz, 1H); 7.29 (d, J=4.4 Hz, 1H); 7.27 (d, J=3.6 Hz, 1H); 7.00 (t, J=5.4 Hz, 1H); 6.75 (d, J=9.2 Hz, 1H); 3.39 (q, J=6.4 Hz, 2H); 3.17 (d, J=4.4 Hz, 2H); 2.37 (t, J=7.0 Hz, 8H); 2.17 (s, 2H); 2.16 (s, 3H); 1.96 (s, 3H); 1.74 (q, J=7.2 Hz, 2H); 1.71-1.57 (m, 12H).

MS: APCI(+ve) 476/477 (M+1).

### EXAMPLE 11

### 2-(1-Adamantyl)-N-(2-{[(2S)-2,3-dihydroxypropyl]amino}quinolin-5-yl)acetamide

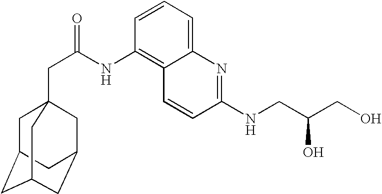

A solution of 2-(1-adamantyl)-N-{2-chloro-quinolin-5-yl}acetamide (Example 2) (75 mg) in 1-methyl-2-pyrrolidinone (2 mL) was treated with (2S)-3-aminopropane-1,2-diol (57 mg) and potassium carbonate (29 mg) following the procedure outlined in Example 6 to give 17 mg of a solid.

¹H NMR (400 MHz, DMSO-d₆) δ 9.64 (s, 1H); 7.98 (d, J=9.2 Hz, 1H); 7.43 (t, J=7.9 Hz, 1H); 7.29 (q, J=7.2 Hz, 2H); 6.86 (d, J=9.2 Hz, 1H); 3.67 (quintet, J=5.5 Hz, 1H); 3.54 (dt, J=13.3, 5.3 Hz, 1H); 3.44-3.26 (m, 5H); 2.18 (s, 2H); 1.96 (s, 3H); 1.73-1.57 (m, 12H).

MS: APCI(+ve) 410 (M+1).

### EXAMPLE 12

### 2-(1-Adamantyl)-N-{2-[(3-hydroxypropyl)amino]-6-methylquinolin-5-yl}acetamide

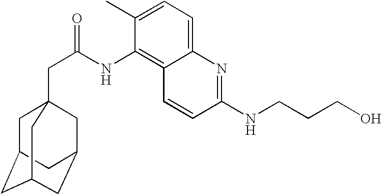

A solution of 2-(1-adamantyl)-N-(2-chloro-6-methylquinolin-5-yl)acetamide (Example 4) (100 mg) in 1-methyl-2-pyrrolidinone (2 mL) was treated with 1-amino-propan-3-ol (398 mg) and potassium carbonate (250 mg) following the procedure outlined in Example 6 to give 40 mg of a solid.

¹H NMR (400 MHz, DMSO-d₆) δ 9.44 (1H, s); 7.74 (1H, d); 7.33 (2H, s); 6.92 (1H, brs); 6.74 (1H, d); 4.68 (1H, brs); 3.49 (2H, t); 3.42 (2H, q); 2.22 (3H, s); 2.18 (2H, s); 1.97 (3H, m); 1.75-1.67 (12H, m); 1.57-1.66 (2H, m).

MS: APCI(+ve) 408 (M+1)

MP: 168-174° C.

### EXAMPLE 13

### 2-(1-Adamantyl)-N-{2-[(2-hydroxyethyl)amino]-6-methylquinolin-5-yl}acetamide

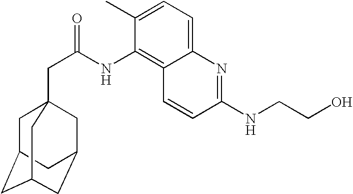

A solution of 2-(1-adamantyl)-N-(2-chloro-6-methylquinolin-5-yl)acetamide (200 mg) in 1-methyl-2-pyrrolidinone (3 mL) was treated with 2-aminoethanol (0.6 mL) and triethylamine (0.4 mL) following the procedure outlined in Example 6 to give 90 mg of a solid isolated as the trifluoroacetic acid salt after purification by reverse phase hplc eluting with 0.1M aqueous trifluoroacetic acid in methanol.

¹H NMR (400 MHz, DMSO-d₆ at 90° C.) δ 9.46 (1H, s); 8.08 (1H, d); 7.59-7.69 (2H, m); 7.15 (1H, d); 3.76-3.68 (2H, m); 3.66-3.58 (2H, m); 2.80 (3H, s); 2.23 (2H, s); 1.98 (3H, m); 1.75-1.55 (12H, m).

MS: APCI(+ve) 394 (M+1)

MP: 103-107° C.

### EXAMPLE 14

### 2-(1-Adamantyl)-N-{2-[[2-(dimethylamino)ethyl] (methyl)amino]-6-methylquinolin-5-yl}acetamide

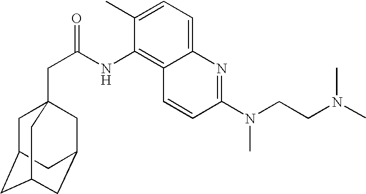

A solution of 2-(1-adamantyl)-N-(2-chloro-6-methylquinolin-5-yl)acetamide (Example 4) (150 mg) in 1-methyl-2-pyrrolidinone (4 mL) was treated with N,N,N′-trimethyl ethylaminediamine (0.2 mL) and potassium carbonate (0.3 g) following the procedure outlined in Example 6 to give 28 mg of a solid isolated as the trifluoroacetic acid salt after purification by reverse phase high pressure liquid chromatography (hplc) eluting with 0.1M aqueous trifluoroacetic acid in methanol.

¹H NMR (400 MHz, DMSO-d₆) δ 9.25 (1H, s); 8.0 (1H, d); 7.44 (2H, m); 7.10 (1H, d); 4.00 (2H, t); 3.38 (2H, t); 3.16 (3H, s); 2.93 (6H, s); 2.83 (2H, s); 2.27 (3H, s); 1.99-1.90 (3H, m); 1.80-1.60 (12H, m).

MS: APCI(+ve) 435 (M+1)

MP: 193-195° C.

### EXAMPLE 15

### 2-(1-Adamantyl)-N-{2-[(2-aminoethyl)amino]quinolin-5-yl}acetamide

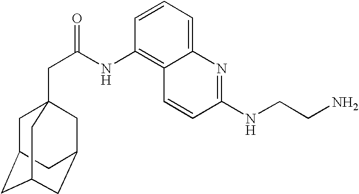

A solution of 2-(1-adamantyl)-N-{2-chloro-quinolin-5-yl}acetamide (Example 2) (71 mg) in ethylenediamine (2 mL) and potassium carbonate (86 mg) was heated to reflux under nitrogen for 2 hours. The solution was concentrated under vacuum and the residue was partitioned between water and dichloromethane. The aqueous was further extracted with dichloromethane and the combined organics were washed with brine, dried over magnesium sulphate, filtered and concentrated to an opalescent residue. This residue was taken into a minimum amount of dichloromethane and ether was added until a cloudy solution appeared then hexane until a precipitate formed. The solid was filtered and dried in a vacuum oven to afford 45 mg of the title compound.

¹H NMR (300 MHz, DMSO-d₆) δ 9.59 (s, 1H); 7.95 (d, J=9.2 Hz, 1H); 7.40 (dd, J=8.3, 7.3 Hz, 1H); 7.29 (dd, J=7.1, 5.2 Hz, 2H); 6.99 (t, J=5.3 Hz, 1H); 6.77 (d, J=9.2 Hz, 1H); 3.39 (q, J=5.9 Hz, 2H); 2.76 (t, J=6.3 Hz, 2H); 2.17 (s, 2H); 1.96 (s, 3H); 1.74-1.55 (m, 12H).

MS: APCI(+ve) 379/380 (M+1).

### EXAMPLE 16

### 2-(1-Adamantyl)-N-{2-[(3-aminopropyl)amino]quinolin-5-yl}acetamide trifluoroacetate

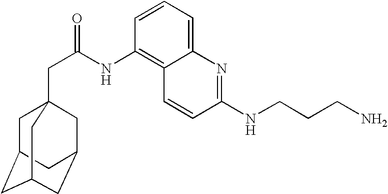

A solution of 2-(1-adamantyl)-N-{2-chloro-quinolin-5-yl}acetamide (Example 2) (75 mg) in 1-methyl-2-pyrrolidinone (2 mL) was treated with propane-1,3-diamine (47 mg) and potassium carbonate (29 mg) and heated to 140° C. under nitrogen for 8 hours in a sealed tube. Water was added to the reaction mixture and the resulting suspension was extracted with dichloromethane (30 mL). The suspension was filtered and the collected solid was boiled in methanol (50 mL) then hot filtered. The filtrate was concentrated under vacuum to give a white yellow solid. This solid was sonicated in ether, filtered, collected by filtration and dried in a vacuum oven at 50° C. to give 30 mg of a solid.

¹H NMR (400 MHz, DMSO-d₆) δ 9.62 (s, 1H); 7.96 (d, J=9.2 Hz, 1H); 7.40 (t, J=7.8 Hz, 1H); 7.30 (dd, J=11.7, 10.5 Hz, 2H); 7.22 (s, 1H); 6.77 (d, J=9.2 Hz, 1H); 3.45 (t, J=6.5 Hz, 2H); 2.76 (t, J=7.0 Hz, 2H); 2.17 (s, 2H); 1.96 (s, 3H); 1.79 (quintet, J=6.3 Hz, 2H); 1.72-1.58 (m, 12H).

MS: APCI(+ve) 393/394 (M+1).

### EXAMPLE 17

### 2-(1-Adamantyl)-N-[2-({2-[(2-hydroxyethyl)amino]ethyl}amino)quinolin-5-yl]acetamide dihydrochloride

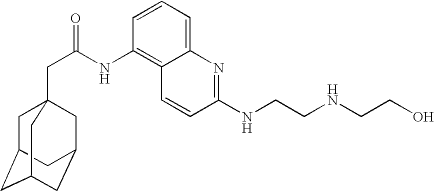

2-(1-Adamantyl)-N-{2-chloro-quinolin-5-yl}acetamide (Example 2) (400 mg) was heated to 130° C. in 1-methyl-2-pyrrolidinone (3 mL) potassium carbonate (156 mg) and 2-[(2-aminoethyl)amino]ethanol (500 μL) for 24 hours. The reaction was cooled to room temperature then partitioned with ethyl acetate (10 mL) and water (10 mL). The aqueous phase was further extracted with ethyl acetate (10 mL) and the combined organic phases were washed with water (20 mL) then brine (20 mL), dried over magnesium sulphate and evaporated to give an orange oil. This oily residue was dissolved in dichloromethane (10 mL), di-(tert-butyl)dicarbonate (500 mg) was added and the solution was stirred for 2 hours under nitrogen. The reaction was concentrated under vacuum to an oil that was purified on silica eluting with methanol in dichloromethane at 0% to 10% in stepwise increments to obtain a white/beige solid. The solid was dissolved in dichloromethane and deprotected with hydrochloric acid at 4M in 1,4-dioxane (700 μL). The solution was stirred for 1 hour under nitrogen, evaporated to dryness, dissolved in the minimum hot methanol and ethyl acetate was added until a precipitate started to form. The cloudy solution was left to stand for 1 hour until a white granular solid had formed. This solid was collected to give 120 mg of the title compound.

¹H NMR (400 MHz, DMSO-d₆) δ 9.80 (s, 1H); 8.37 (d, J=9.5 Hz, 1H); 8.01 (d, J=7.9 Hz, 1H); 7.66 (t, J=8.1 Hz, 1H); 7.58 (d, J=7.5 Hz, 1H); 7.21 (d, J=9.5 Hz, 1H); 4.09 (t, J=6.2 Hz, 2H); 3.74 (t, J=5.4 Hz, 2H); 3.33 (t, J=6.2 Hz, 2H); 3.11 (t, J=5.4 Hz, 2H); 2.24 (s, 2H); 1.96 (s, 3H); 1.71 (s, 6H); 1.67 (q, J=12.6 Hz, 6H).

MS: APCI(+ve) 423 (M+1).

### EXAMPLE 18

### 2-(1-Adamantyl)-N-{2-[(2-aminoethyl)(2-hydroxyethyl)amino]quinolin-5-yl}acetamide dihydrochloride

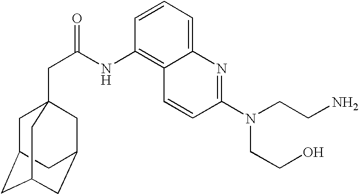

From the reaction described in Example 17 was isolated 20 mg of a second product that was characterised as being 2-(1-adamantyl)-N-{2-[(2-aminoethyl)(2-hydroxyethyl)amino]quinolin-5-yl}acetamide.

¹H NMR (300 MHz, DMSO-d₆) δ 10.17 (s, 1H); 8.55-8.18 (m, 4H); 7.74 (t, J=8.1 Hz, 3H); 7.64 (d, J=7.7 Hz, 2H); 4.19 (s, 2H); 3.98 (s, 2H); 3.73 (s, 2H); 3.20 (s, 2H); 2.25 (s, 2H); 1.96 (s, 3H); 1.77-1.53 (m, 12H).

MS: APCI(+ve) 423/424 (M+1).

### EXAMPLES 19 TO 69

A series of compound were prepared in a combinatorial chemistry format as follows.

A box of selected starting aldehyde (0.1 μmol) was dissolved in 1-methyl-2-pyrrolidinone (1 mL in each well). 55 μl were transferred to a new box previously loaded with 2-(1-adamantyl)-N-{2-[(2-aminoethyl)amino]quinolin-5-yl}acetamide (Example 15) (1.89 mg in each well) dissolved in N-methylpyrrolidinone (20 μL in each well). Acetic acid (4 μL in each well) was added and the box was gently shaken for two hours. Sodium cyanoborohydride (in excess) was added and the box was gently shaken for a further 12 hours. Isopropylamine (100 μL in each well) was then added and the solvents were evaporated in vacuum in a Genevac HT-8 Atlas Evaporator. The residues were dissolved in dimethyl sulfoxide (100 μL in each well) and purified by mass directed purification.

| | | | Theoretical | (M + H) |
|---|---|---|---|---|
| Example No./Structure | | Name | mol. wt. | collected |
| 19 | 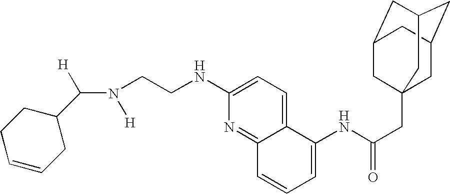 | 2-(1-Adamantyl)-N-[2-({2-{(cyclohex-3-en-1-ylmethyl)amino]ethyl}-amino)quinolin-5-yl]acetamide | 472.3202 | 473.422 |
| 20 | 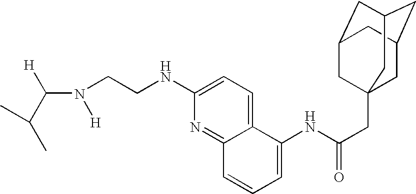 | 2-(1-Adamantyl)-N-(2-{[2-(isobutylamino)ethyl]-amino}quinolin-5-yl)acetamide | 434.3045 | 435.441 |
| 21 | 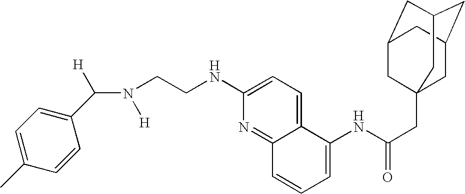 | 2-(1-Adamantyl)-N-[2-({2-[(4-methylbenzyl)-amino]ethyl}amino)-quinolin-5-yl]acetamide | 482.3045 | 483.412 |
| 22 | 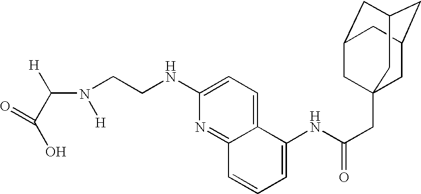 | {[2-({5-[(1-adamantylacetyl)amino]quinolin-2-yl}amino)ethyl]amino)acetic acid | 436.2474 | 437.37 |
| 23 | 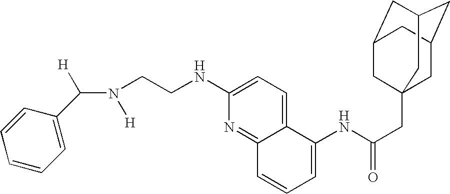 | 2-(1-Adamantyl)-N-(2-{[2-(benzylamino)ethyl]-amino}quinolin-5-yl)acetamide | 468.2889 | 469.4 |
| 24 | 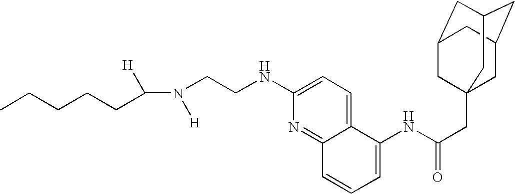 | 2-(1-Adamantyl)-N-(2-{[2-(hexylamino)ethyl]-amino}quinolin-5-yl)acetamide | 462.3358 | 463.448 |
| 25 | 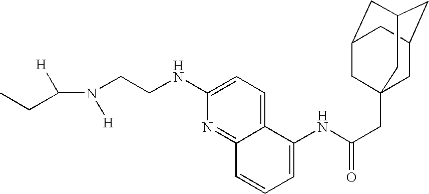 | 2-(1-Adamantyl)-N-(2-{[2-(propylamino)ethyl]-amino}quinolin-5-yl)acetamide | 420.2889 | 421.399 |
| 26 | 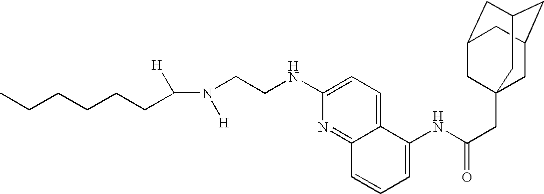 | 2-(1-Adamantyl)-N-(2-{[2-(heptylamino)ethyl]-amino}quinolin-5-yl)acetamide | 476.3515 | 477.452 |
| 27 | 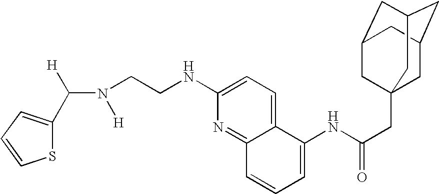 | 2-(1-Adamantyl)-N-[2-({2-[(thien-2-ylmethyl)amino]-ethyl}amino)quinolin-5-yl]acetamide | 474.2453 | 473.351 |
| 28 | 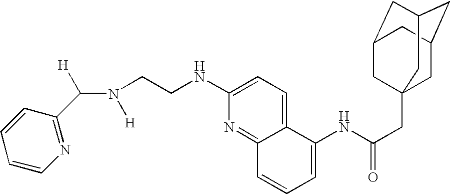 | 2-(1-Adamantyl)-N-[2-({2-[(pyridin-2-ylmethyl)amino]-ethyl}amino)quinolin-5-yl]acetamide | 469.2841 | 470.4 |
| 29 | 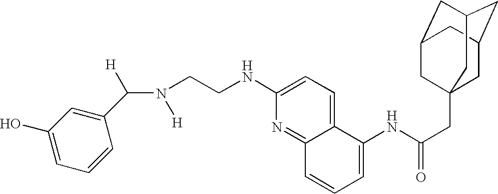 | 2-(1-Adamantyl)-N-[2-({2-[(3-hydroxybenzyl)-amino]ethyl}amino)-quinolin-5-yl]acetamide | 484.2838 | 485.232 |
| 30 | 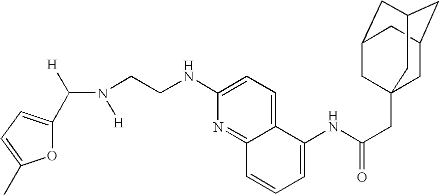 | 2-(1-Adamantyl)-N-{2-[(2-{[(5-methyl-2-furyl)methyl]amino}ethyl)amino]quinolin-5-yl}acetamide | 472.2838 | 473.391 |
| 31 | 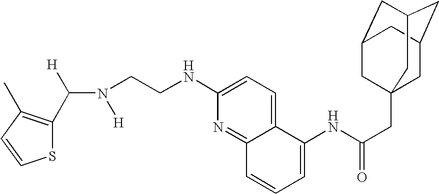 | 2-(1-Adamantyl)-N-{2-[(2-{[(3-methylthien-2-yl)methyl]amino}ethyl)-amino]quinolin-5-yl}acetamide | 488.261 | 489.363 |
| 32 | 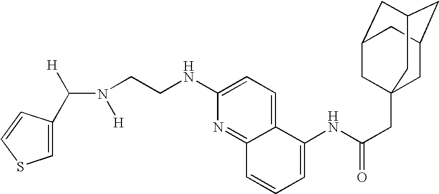 | 2-(1-Adamantyl)-N-[2-({2-[(thien-3-ylmethyl)-amino]ethyl}amino)-quinolin-5-yl]acetamide | 474.2453 | 473.351 |
| 33 | 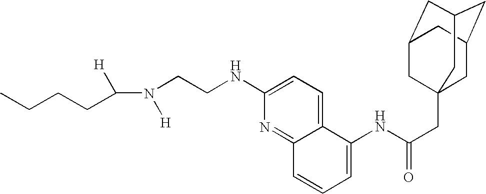 | 2-(1-Adamantyl)-N-(2-{[2-(pentylamino)ethyl]-amino}quinolin-5-yl)acetamide | 448.3202 | 449.445 |
| 34 | 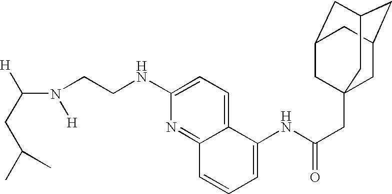 | 2-(1-Adamantyl)-N-(2-{[2-(isopentylamino)ethyl]-amino}quinolin-5-yl)acetamide | 448.3202 | 449.421 |
| 35 | 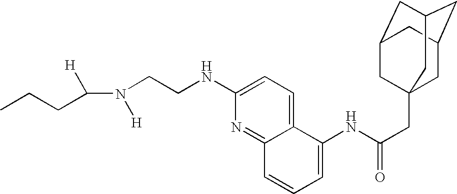 | 2-(1-Adamantyl)-N-(2-{[2-(butylamino)ethyl]-amino}quinolin-5-yl)acetamide | 434.3045 | 435.426 |
| 36 | 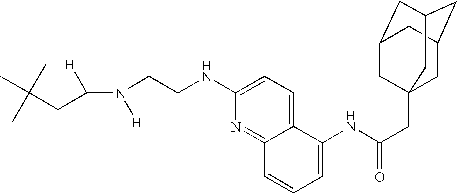 | 2-(1-Adamantyl)-N-[2-({2-[(3,3-dimethylbutyl)-amino]ethyl}amino)-quinolin-5-yl]acetamide | 462.3358 | 463.448 |
| 37 | 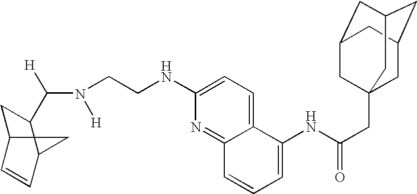 | 2-(1-Adamantyl)-N-[2-({2-[(bicyclo[2.2.1]hept-5-en-2-ylmethyl)amino]-ethyl}amino)quinolin-5-yl]acetamide | 484.3202 | 485.419 |
| 38 | 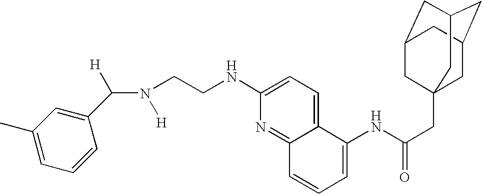 | 2-(1-Adamantyl)-N-[2-({2-[(3-methylbenzyl)-amino]ethyl}amino)-quinolin-5-yl]acetamide | 482.3045 | 483.404 |
| 39 | 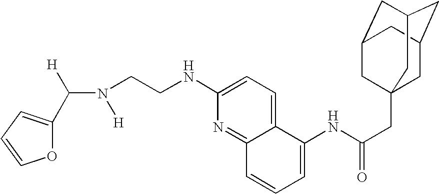 | 2-(1-Adamantyl)-N-[2-({2-[(2-furylmethyl)-amino]ethyl}amino)-quinolin-5-yl]acetamide | 458.2682 | 459.387 |
| 40 | 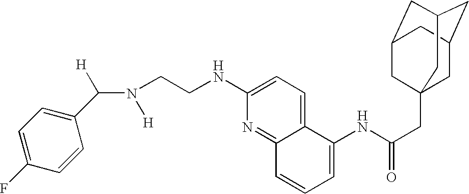 | 2-(1-Adamantyl)-N-[2-({2-[(4-fluorobenzyl)-amino]ethyl}amino)-quinolin-5-yl]acetamide | 486.2795 | 487.38 |
| 41 | 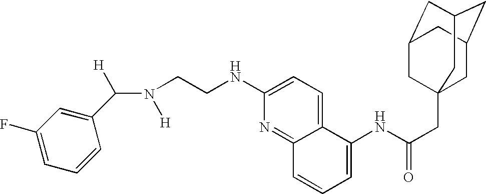 | 2-(1-Adamantyl)-N-[2-({2-[(3-fluorobenzyl)-amino]ethyl}amino)-quinolin-5-yl]acetamide | 486.2795 | 487.38 |
| 42 | 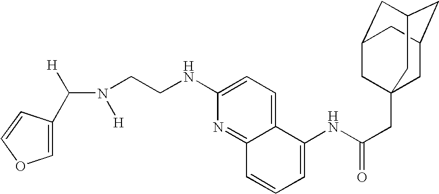 | 2-(1-Adamantyl)-N-[2-({2-[(3-furylmethyl)-amino]ethyl}amino)-quinolin-5-yl]acetamide | 458.2682 | 459.387 |
| 43 | 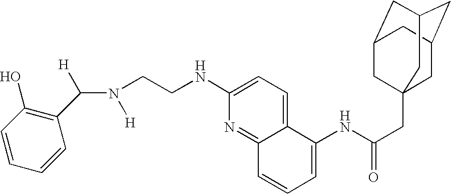 | 2-(1-Adamantyl)-N-[2-({2-[(2-hydroxybenzyl)-amino]ethyl}amino)-quinolin-5-yl]acetamide | 484.2838 | 485.372 |
| 44 | 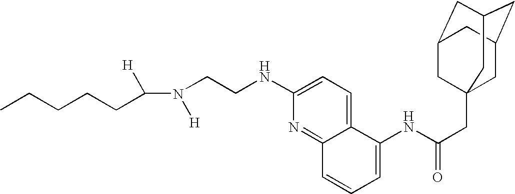 | 2-(1-Adamantyl)-N-[2-({2-[(2E)-hex-2-enylamino]ethyl}amino)-quinolin-5-yl]acetamide | 460.3202 | 461.426 |
| 45 | 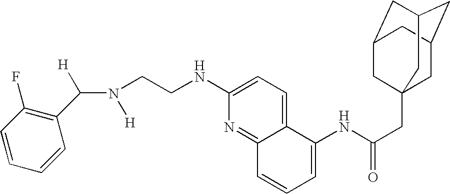 | 2-(1-Adamantyl)-N-[2-({2-[(2-fluorobenzyl)-amino]ethyl}amino)-quinolin-5-yl]acetamide | 486.2795 | 487.387 |
| 46 | 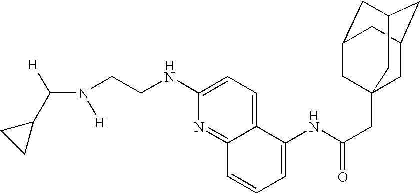 | 2-(1-Adamantyl)-N-[2-({2-[(cyclopropylmethyl)-amino]ethyl}amino)-quinolin-5-yl]acetamide | 432.2889 | 433.403 |
| 47 | 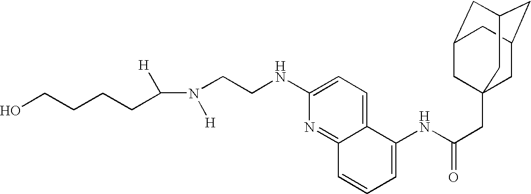 | 2-(1-Adamantyl)-N-[2-({2-[(5-hydroxypentyl)-amino]ethyl}amino)-quinolin-5-yl]acetamide | 464.3151 | 465.424 |
| 48 | 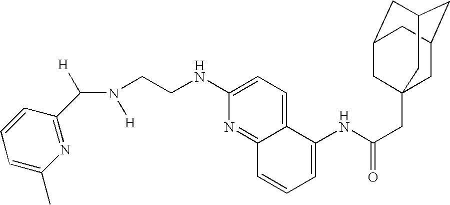 | 2-(1-Adamantyl)-N-{2-[(2-{[(6-methylpyridin-2-yl)methyl]amino}ethyl)-amino]quinolin-5-yl}acetamide | 483.2998 | 484.404 |
| 49 | 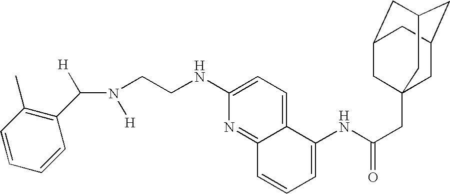 | 2-(1-Adamantyl)-N-[2-({2-[(2-methylbenzyl)amino]ethyl}amino)quinolin-5-yl]acetamide | 482.3045 | 483.412 |
| 50 | 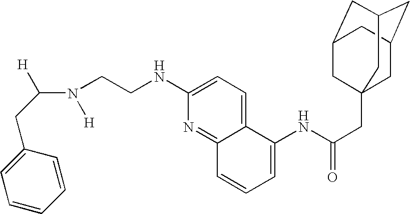 | 2-(1-Adamantyl)-N-[2-({2-[(2-phenylethyl)-amino]ethyl}amino)-quinolin-5-yl]acetamide | 482.3045 | 483.404 |
| 51 | 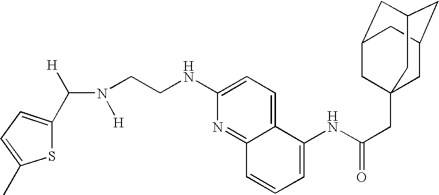 | 2-(1-Adamantyl)-N-{2-[(2-{[(5-methylthien-2-yl)methyl]amino}ethyl)-amino]quinolin-5-yl}acetamide | 488.261 | 489.223 |
| 52 | 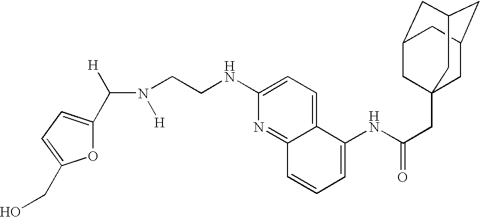 | 2-(1-Adamantyl)-N-(2-{[2-({[5-(hydroxymethyl)-2-furyl]methyl}amino)-ethyl]amino)quinolin-5-yl)acetamide | 488.2787 | 489.371 |
| 53 | 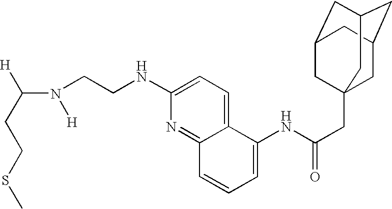 | 2-(1-Adamantyl)-N-{2-[(2-{[3-(methylthio)propyl]-amino}ethyl)amino]-quinolin-5-yl}acetamide | 466.2766 | 467.377 |
| 54 | 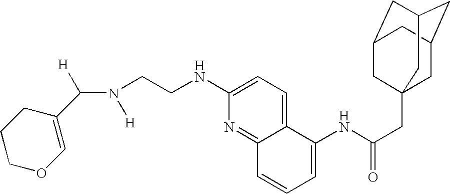 | 2-(1-Adamantyl)-N-[2-({2-[(3,4-dihydro-2H-pyran-5-ylmethyl)amino]-ethyl}amino)quinolin-5-yl]acetamide | 474.2994 | 475.406 |
| 55 | 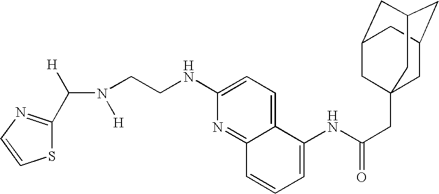 | 2-(1-Adamantyl)-N-[2-({2-[(1,3-thiazol-2-ylmethyl)amino]ethyl}-amino)quinolin-5-yl]acetamide | 475.2406 | 476.335 |
| 56 | 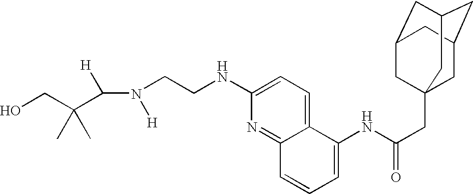 | 2-(1-Adamantyl)-N-[2-({2-[(3-hydroxy-2,2-dimethylpropyl)amino]-ethyl}amino)quinolin-5-yl]acetamide | 464.3151 | 465.432 |
| 57 | 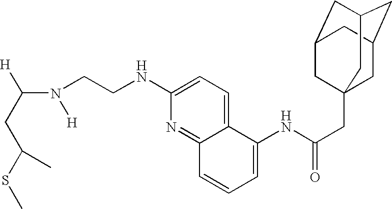 | 2-(1-Adamantyl)-N-{2-[(2-{[3-(methylthio)-butyl]amino}ethyl)-amino]quinolin-5-yl}acetamide | 480.2923 | 481.397 |
| 58 | 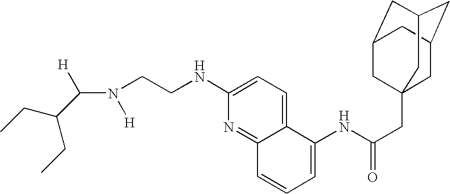 | 2-(1-Adamantyl)-N-[2-({2-[(2-ethylbutyl)amino]-ethyl}amino)quinolin-5-yl]acetamide | 462.3358 | 463.441 |
| 59 | 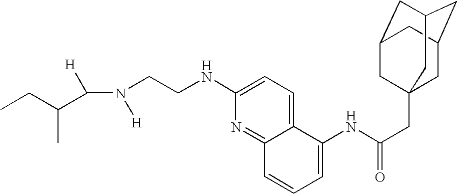 | 2-(1-Adamantyl)-N-{2-[(2-{[(2E)-2-methylbut-2-enyl)amino}ethyl)amino]-quinolin-5-yl}acetamide | 446.3045 | 447.422 |
| 60 | 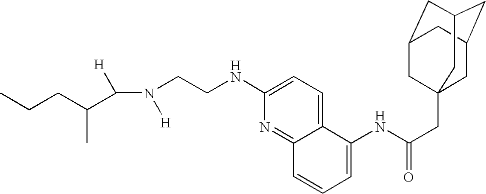 | 2-(1-Adamantyl)-N-{2-[(2-{[(2E)-2-methylpent-2-enyl]amino}ethyl)amino]-quinolin-5-yl}acetamide | 460.3202 | 461.441 |
| 61 | 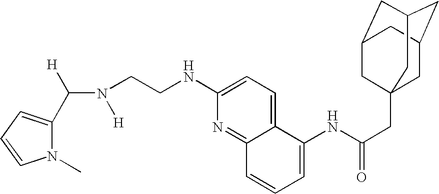 | 2-(1-Adamantyl)-N-{2-[(2-{[(1-methyl-1H-pyrrol-2-yl)methyl]-amino}ethyl)amino]-quinolin-5-yl}acetamide | 471.2998 | 472.407 |
| 62 | 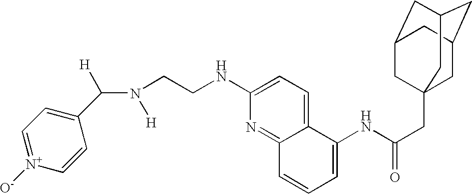 | 2-(1-Adamantyl)-N-{2-[(2-{[(1-oxipyridin-4-yl)methyl]amino}ethyl)-amino]quinolin-5-yl}acetamide | 485.2791 | 486.38 |
| 63 | 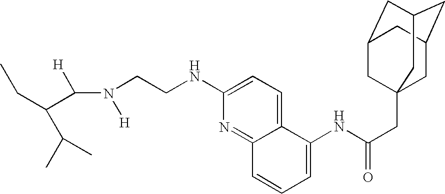 | 2-(1-Adamantyl)-N-[2-({2-[(2-ethyl-3-methylbutyl)amino]ethyl}-amino)quinolin-5-yl]acetamide | 476.3515 | 477.46 |
| 64 | 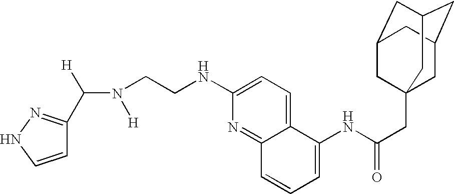 | 2-(1-Adamantyl)-N-[2-({2-(1H-pyrazol-3-ylmethyl)amino]ethyl}-amino)quinolin-5-yl]acetamide | 458.2794 | 459.387 |
| 65 | 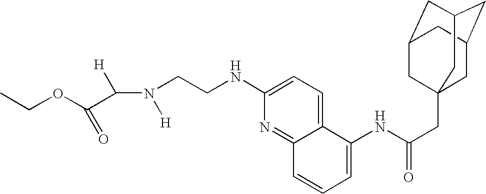 | Ethyl{[2-({5-[(1-adamantylacetyl)amino]-quinolin-2-yl}amino)-ethyl]amino}acetate | 464.2787 | 465.385 |
| 66 | 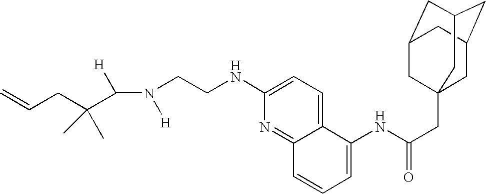 | 2-(1-Adamantyl)-N-[2-({2-[(2,2-dimethylpent-4-enyl)amino]ethyl}amino)-quinolin-5-yl]acetamide | 474.3358 | 475.429 |
| 67 | 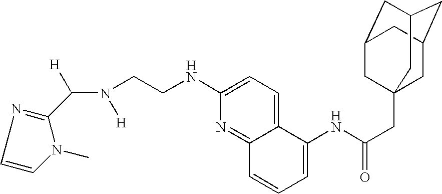 | 2-(1-Adamantyl)-N-{2-[(2-{[(1-methyl-1H-imidazol-2-yl)methyl]-amino}ethyl)amino]-quinolin-5-yl}acetamide | 472.295 | 473.407 |
| 68 | 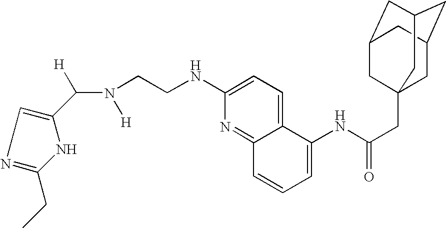 | 2-(1-Adamantyl)-N-{2-[(2-{[(2-ethyl-1H-imidazol-5-yl)methyl]-amino}ethyl)amino]-quinolin-5-yl}acetamide | 486.3107 | 487.419 |
| 69 | 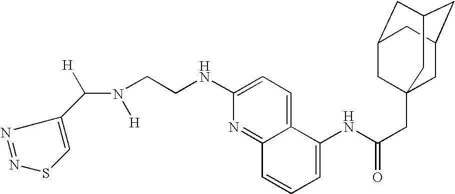 | 2-(1-Adamantyl)-N-[2-({2-[(1,2,3-thiadiazol-4-ylmethyl)amino]ethyl}-amino)quinolin-5-yl]acetamide | 476.2358 | 477.335 |

### EXAMPLES 70 TO 118

A series of compound were prepared in a combinatorial chemistry format as follows. A box of selected starting aldehyde (0.1 mmol) was dissolved in N-methylpyrrolidinone (1 mL in each well). 55 μL were transferred to a new box previously loaded with 2-(1-adamantyl)-N-{2-[(3-aminopropyl)amino]quinolin-5-yl}acetamide (Example 16) (1.96 mg in each well) dissolved in N-methylpyrrolidinone (20 μL in each well). Acetic acid (4 μL in each well) was added and the box was gently shaken for two hours. Sodium cyanoborohydride (in excess) was added and the box was gently shaken for a further 12 hours. Isopropylamine (100 μL in each well) was then added and the solvents were evaporated in vacuum in a Genevac HT-8 Atlas Evaporator. The residues were dissolved in dimethyl sulfoxide (100 μL in each well) and purified by mass directed purification.

| | | | Theoretical | (M + H) |
|---|---|---|---|---|
| Example No./Structure | | Name | mol. wt. | collected |
| 70 | 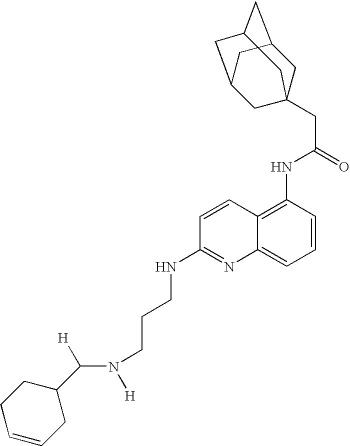 | 2-(1-Adamantyl)-N-[2-({3-[(cyclohex-3-en-1-ylmethyl)amino]propyl}-amino)quinolin-5-yl]acetamide | 486.3358 | 487.403 |
| 71 | 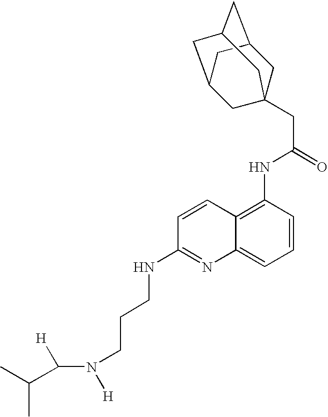 | 2-(1-Adamantyl)-N-(2-{[3-(isobutylamino)-propyl]amino}quinolin-5-yl)acetamide | 448.3202 | 449.398 |
| 72 | 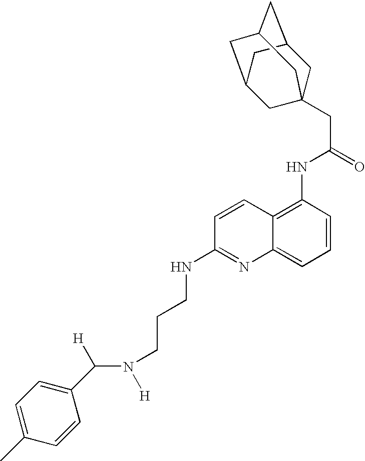 | 2-(1-Adamantyl)-N-[2-({3-[(4-methylbenzyl)-amino]propyl}-amino)quinolin-5-yl]acetamide | 496.3202 | 497.408 |
| 73 | 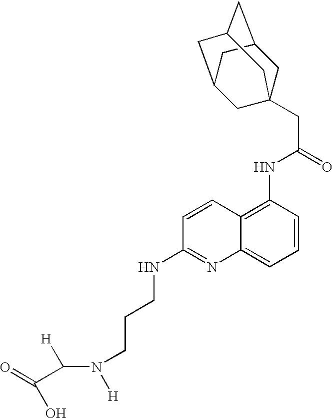 | {[3-({5-[(1-Adamantylacetyl)-amino]quinolin-2-yl}amino)propyl]-amino}acetic acid | 450.2631 | 451.358 |
| 74 | 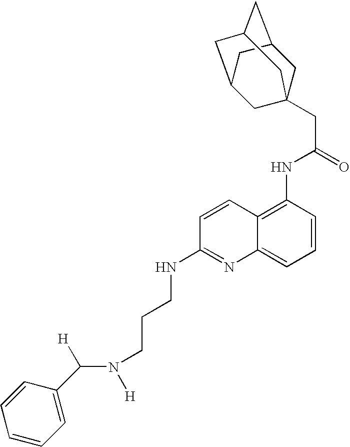 | 2-(1-Adamantyl)-N-(2-{[3-(benzylamino)-propyl]amino}quinolin-5-yl)acetamide | 482.3045 | 483.388 |
| 75 | 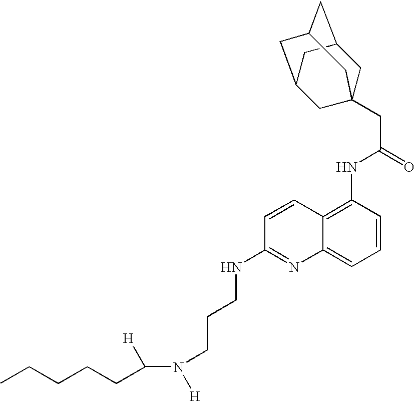 | 2-(1-Adamantyl)-N-(2-{[3-(hexylamino)-propyl]amino}quinolin-5-yl)acetamide | 476.3515 | 477.429 |
| 76 | 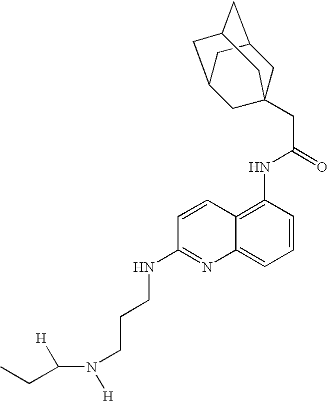 | 2-(1-Adamantyl)-N-(2-{[3-(propylamino)-propyl]amino}quinolin-5-yl)acetamide | 434.3045 | 435.402 |
| 77 | 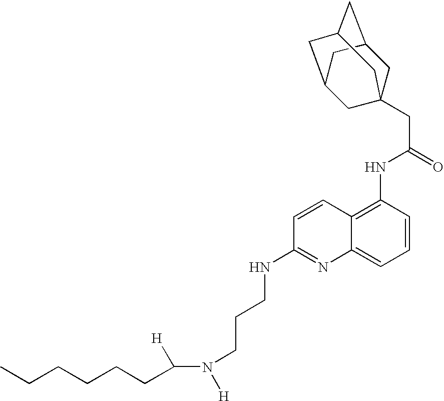 | 2-(1-Adamantyl)-N-(2-{[3-(heptylamino)-propyl]amino}quinolin-5-yl)acetamide | 490.3671 | 491.449 |
| 78 | 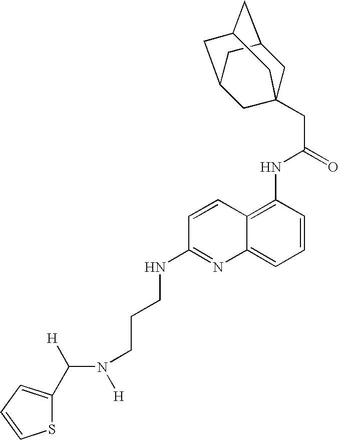 | 2-(1-Adamantyl)-N-[2-({3-[(thien-2-ylmethyl)amino]-propyl}amino)quinolin-5-yl]acetamide | 488.261 | 489.348 |
| 79 | 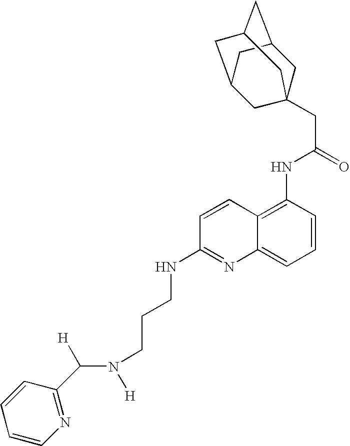 | 2-(1-Adamantyl)-N-[2-({3-[(pyridin-2-ylmethyl)amino]-propyl)amino)quinolin-5-yl]acetamide | 483.2998 | 484.388 |
| 80 | 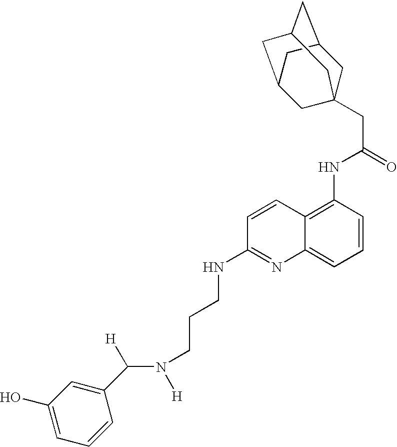 | 2-(1-Adamantyl)-N-[2-({3-[(3-hydroxybenzyl)-amino]propyl}-amino)quinolin-5-yl]acetamide | 498.2994 | 499.369 |
| 81 | 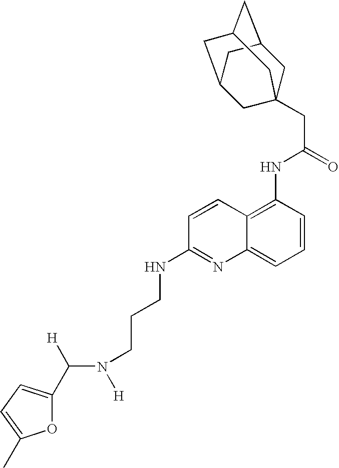 | 2-(1-Adamantyl)-N-{2-[(3-{[(5-methyl-2-furyl)methyl]-amino}propyl)-amino]quinolin-5-yl}acetamide | 486.2994 | 487.387 |
| 82 | 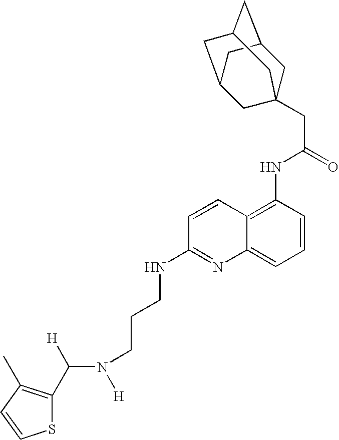 | 2-(1-Adamantyl)-N-{2-[(3- {[(3-methylthien-2-yl)methyl]amino}-propyl)amino]quinolin-5-yl}acetamide | 502.2766 | 503.352 |
| 83 | 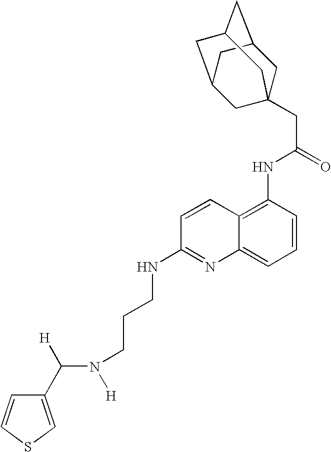 | 2-(1-Adamantyl)-N-[2-({3-[(thien-3-ylmethyl)amino]-propyl}amino)quinolin-5-yl]acetamide | 488.261 | 489.34 |
| 84 | 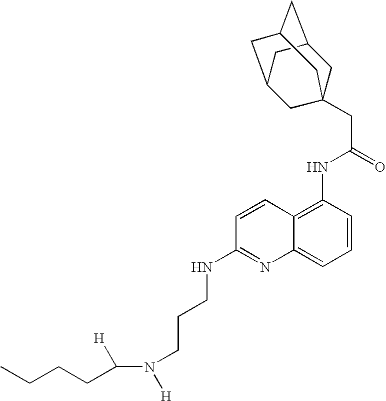 | 2-(1-Adamantyl)-N-(2-{[3-(pentylamino)-propyl]amino)quinolin-5-yl)acetamide | 462.3358 | 463.425 |
| 85 | 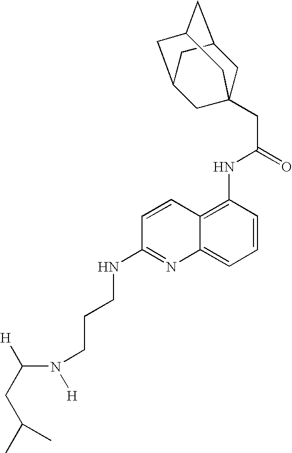 | 2-(1-Adamantyl)-N-(2-{[3-(isopentylamino)-propyl]amino}quinolin-5-yl)acetamide | 462.3358 | 463.409 |
| 86 | 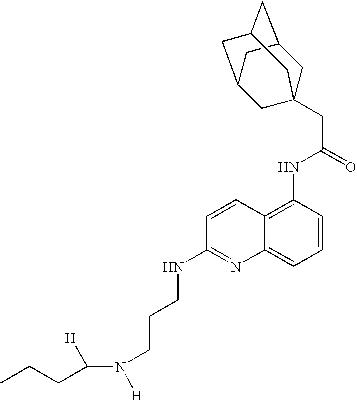 | 2-(1-Adamantyl)-N-(2-{[3-(butylamino)propyl]-amino}quinolin-5-yl)acetamide | 448.3202 | 449.406 |
| 87 | 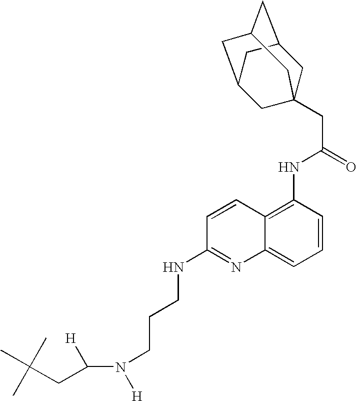 | 2-(1-Adamantyl)-N-[2-({3-[(3,3-dimethylbutyl)-amino]propyl}-amino)quinolin-5-yl]acetamide | 476.3515 | 477.437 |
| 88 | 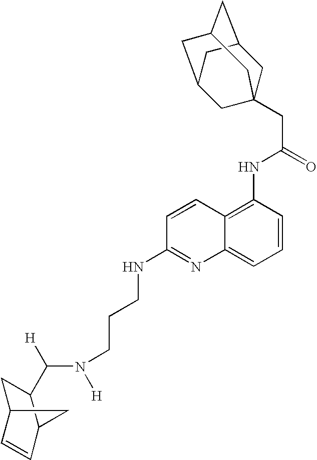 | 2-(1-Adamantyl)-N-[2-({3-[(bicyclo[2.2.1]hept-5-en-2-ylmethyl)-amino]propyl}-amino)quinolin-5-yl]acetamide | 498.3358 | 499.416 |
| 89 | 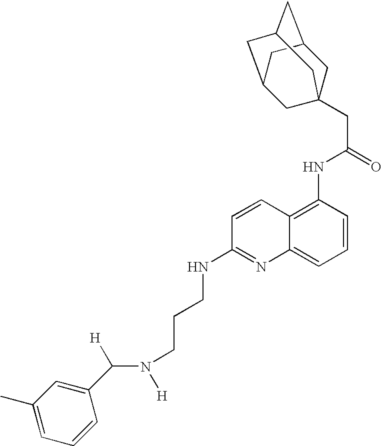 | 2-(1-Adamantyl)-N-[2-({3-[(3-methylbenzyl)-amino]propyl}-amino)quinolin-5-yl]acetamide | 496.3202 | 497.401 |
| 90 | 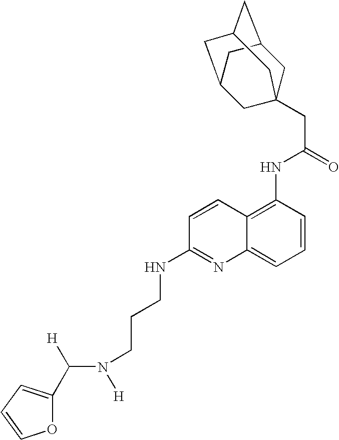 | 2-(1-Adamantyl)-N-[2-({3-[(2-furylmethyl)-amino]propyl}amino)-quinolin-5-yl]acetamide | 472.2838 | 473.391 |
| 91 | 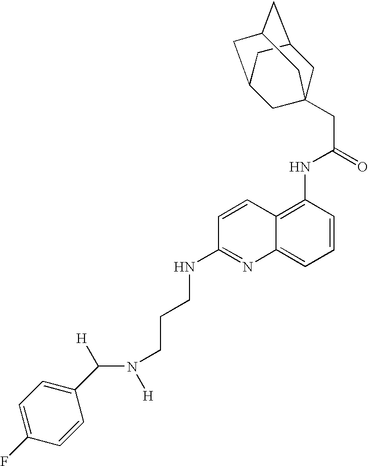 | 2-(1-Adamantyl)-N-[2-({3-[(4-fluorobenzyl)-amino]propyl}amino)-quinolin-5-yl]acetamide | 500.2951 | 501.376 |
| 92 | 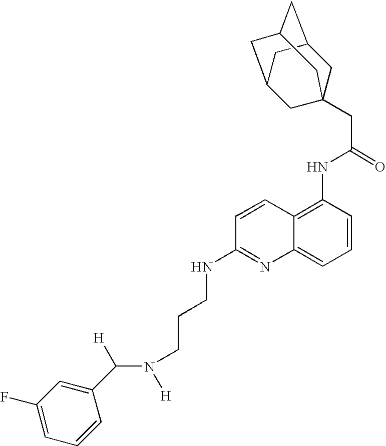 | 2-(1-Adamantyl)-N-[2-({3-[(3-fluorobenzyl)-amino]propyl}amino)-quinolin-5-yl]acetamide | 500.2951 | 501.376 |
| 93 | 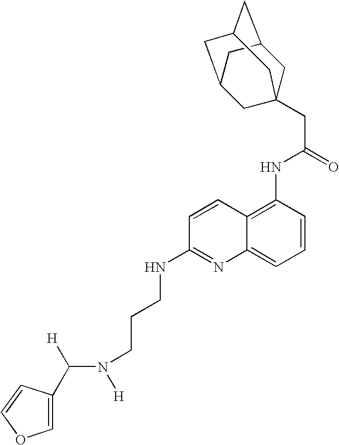 | 2-(1-Adamantyl)-N-[2-({3-[(3-furylmethyl)-amino]propyl}amino)-quinolin-5-yl]acetamide | 472.2838 | 473.36 |
| 94 | 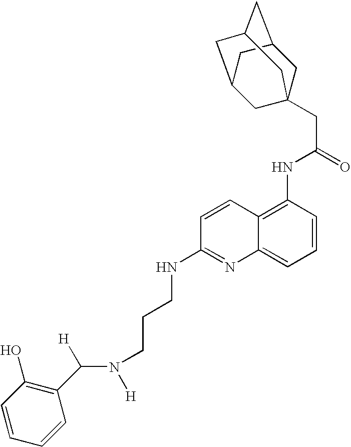 | 2-(1-Adamantyl)-N-[2-({3-[(2-hydroxybenzyl)-amino]propyl}-amino)quinolin-5-yl]acetamide | 498.2994 | 499.353 |
| 95 | 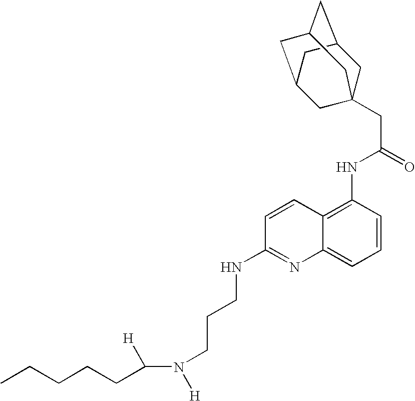 | 2-(1-Adamantyl)-N-[2-({3-[(2E)-hex-2-enylamino]propyl}-amino)quinolin-5-yl]acetamide | 474.3358 | 477.437 |
| 96 | 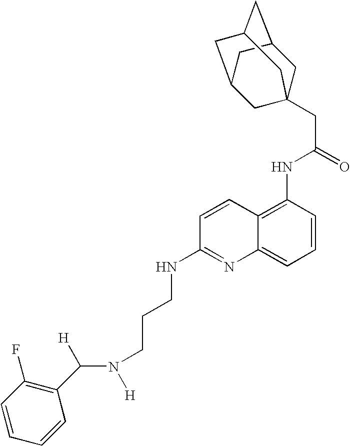 | 2-(1-Adamantyl)-N-[2-({3-[(2-fluorobenzyl)-amino]propyl}amino)-quinolin-5-yl]acetamide | 500.2951 | 501.361 |
| 97 | 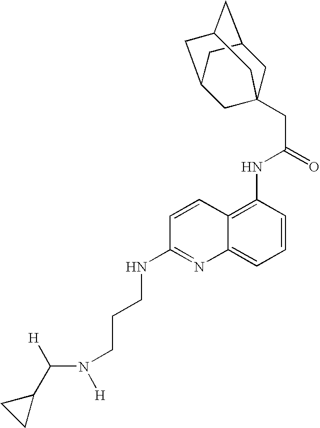 | 2-(1-Adamantyl)-N-[2-({3-[(cyclopropylmethyl)-amino]propyl}-amino)quinolin-5-yl]acetamide | 446.3045 | 447.414 |
| 98 | 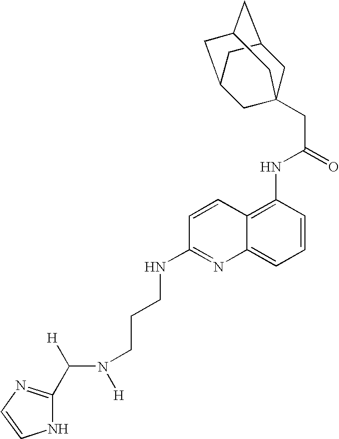 | 2-(1-Adamantyl)-N-[2-({3-(1H-imidazol-2-ylmethyl)amino]-propyl}amino)quinolin-5-yl]acetamide | 472.295 | 473.203 |
| 99 | 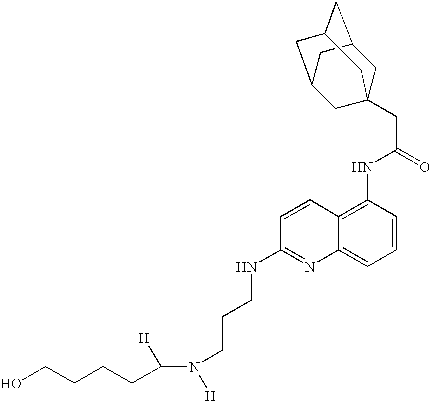 | 2-(1-Adamantyl)-N-[2-({3-[(5-hydroxypentyl)-amino]propyl}amino)-quinolin-5-yl]acetamide | 478.3307 | 479.413 |
| 100 | 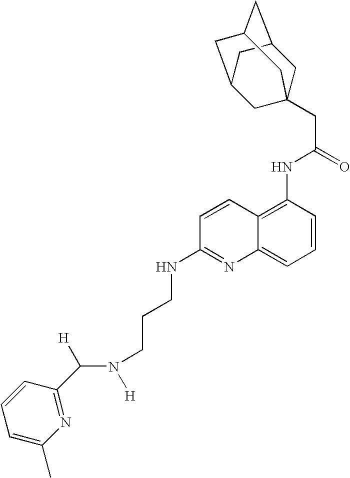 | 2-(1-Adamantyl)-N-{2-[(3-{[(6-methylpyridin-2-yl)methyl]amino}-propyl)amino]quinolin-5-yl}acetamide | 497.3154 | 498.385 |
| 101 | 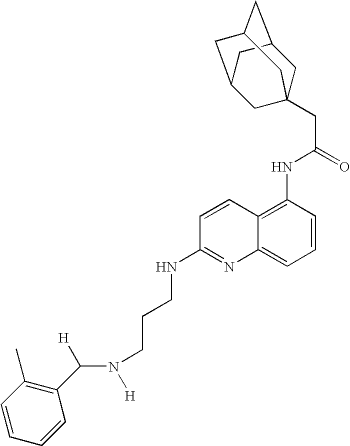 | 2-(1-Adamantyl)-N-[2-({3-[(2-methylbenzyl)-amino]propyl}amino)-quinolin-5-yl]acetamide | 496.3202 | 497.385 |
| 102 | 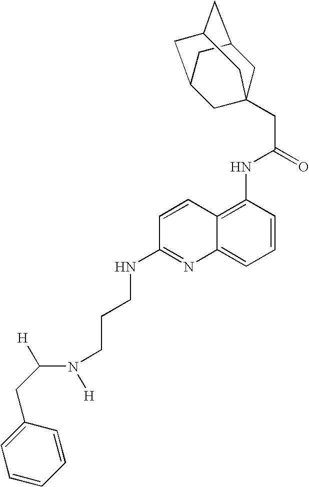 | 2-(1-Adamantyl)-N-[2-({3-[(2-phenylethyl)-amino]propyl}amino)-quinolin-5-yl]acetamide | 496.3202 | 497.393 |
| 103 | 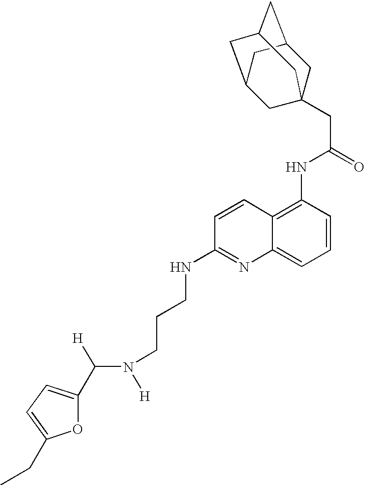 | 2-(1-Adamantyl)-N-{2-[(3-{[(5-ethyl-2-furyl)methyl]amino}-propyl)amino]quinolin-5-yl}acetamide | 500.3151 | 501.384 |
| 104 | 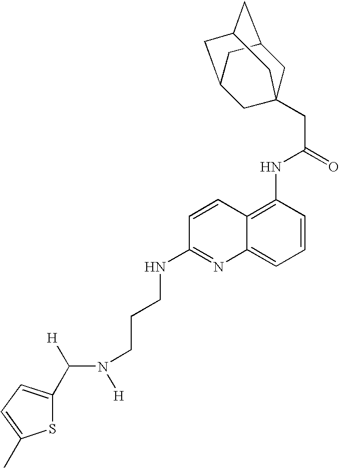 | 2-(1-Adamantyl)-N-{2-[(3-{[(5-methylthien-2-yl)methyl]amino}-propyl)amino]quinolin-5-yl}acetamide | 502.2766 | 503.149 |
| 105 | 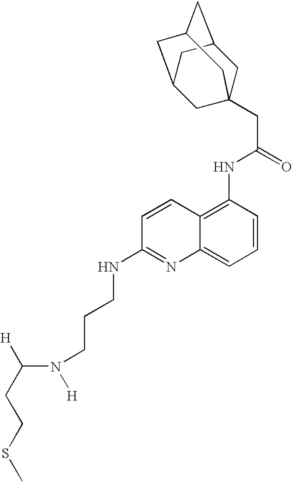 | 2-(1-Adamantyl)-N-{2-[(3-{[3-(methylthio)-propyl]amino}propyl)-amino]quinolin-5-yl}acetamide | 480.2923 | 481.365 |
| 106 | 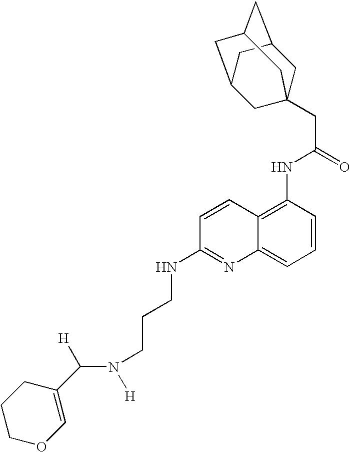 | 2-(1-Adamantyl)-N-[2-({3-[(3,4-dihydro-2H-pyran-5-ylmethyl)-amino]propyl}amino)-quinolin-5-yl]acetamide | 488.3151 | 489.395 |
| 107 | 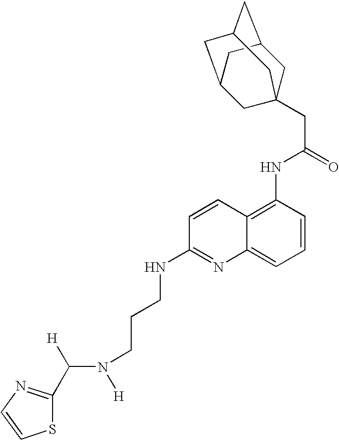 | 2-(1-Adamantyl)-N-[2-({3-[(1,3-thiazol-2-ylmethyl)amino]-propyl]amino)quinolin-5-yl]acetamide | 489.2562 | 490.324 |
| 108 | 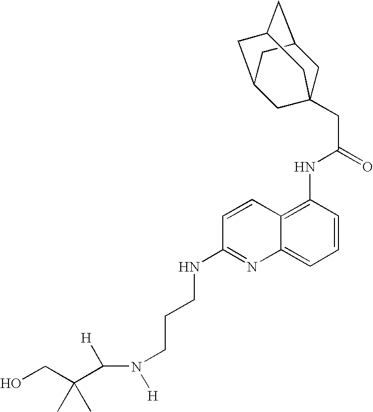 | 2-(1-Adamantyl)-N-[2-({3-[(3-hydroxy-2,2-dimethylpropyl)-amino]propyl}amino)-quinolin-5-yl]acetamide | 478.3307 | 479.413 |
| 109 | 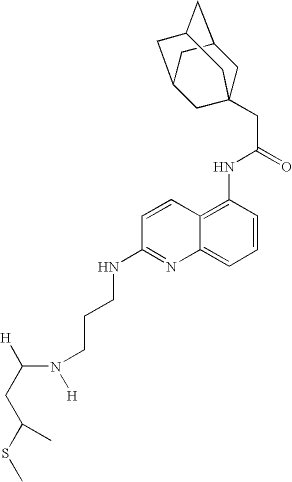 | 2-(1-Adamantyl)-N-{2-[(3-{[3-(methylthio)-butyl]amino}propyl)-amino]quinolin-5-yl}acetamide | 494.3079 | 495.393 |
| 110 | 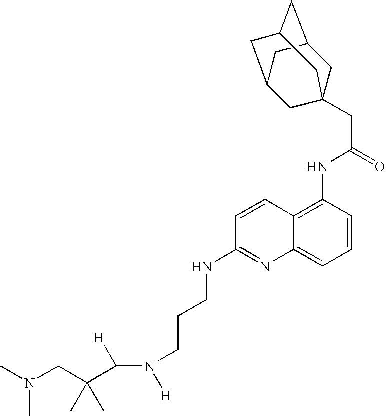 | 2-(1-Adamantyl)-N-{2-[(3-{(3-(dimethylamino)-2,2-dimethylpropyl]-amino}propyl)amino]-quinolin-5-yl}acetamide | 505.378 | 506.446 |
| 111 | 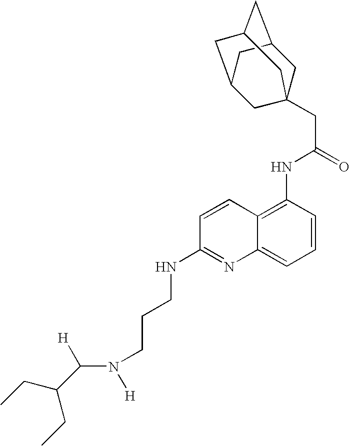 | 2-(1-Adamantyl)-N-[2-({3-[(2-ethylbutyl)-amino]propyl}amino)-quinolin-5-yl]acetamide | 476.3515 | 477.437 |
| 112 | 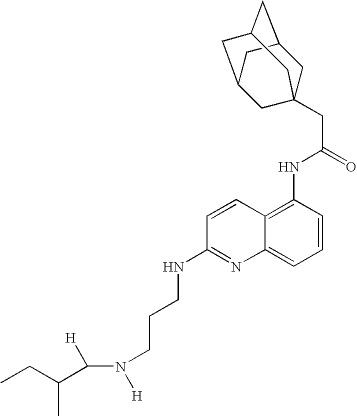 | 2-(1-Adamantyl)-N-{2-[(3-{[(2E)-2-methylbut-2-enyl]amino}propyl)-amino]quinolin-5-yl}acetamide | 460.3202 | 461.41 |
| 113 | 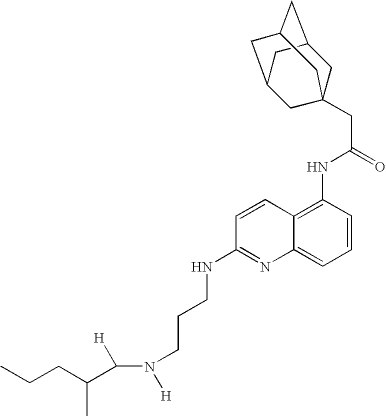 | 2-(1-Adamantyl)-N-{2-[(3-{[(2E)-2-methylpent-2-enyl]amino}propyl)-amino]quinolin-5-yl}acetamide | 474.3358 | 475.414 |
| 114 | 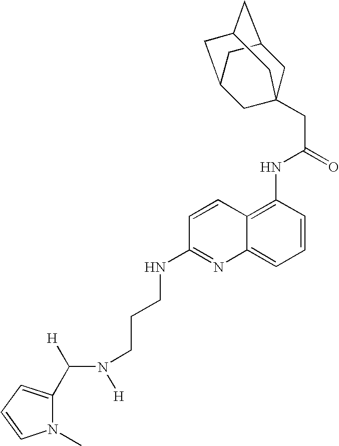 | 2-(1-Adamantyl}-N-{2-[(3-{[(1-methyl-1H-pyrrol-2-yl)methyl]-amino}propyl)amino]-quinolin-5-yl}acetamide | 485.3154 | 486.388 |
| 115 | 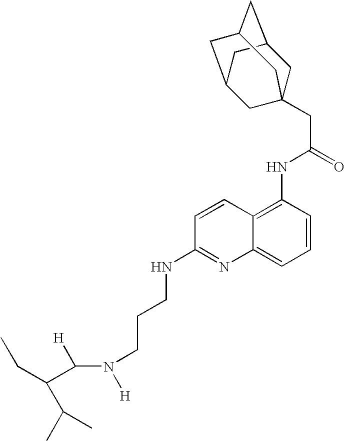 | 2-(1-Adamantyl)-N-[2-({3-[(2-ethyl-3-methylbutyl)amino]-propyl}amino)quinolin-5-yl]acetamide | 490.3671 | 491.457 |
| 116 | 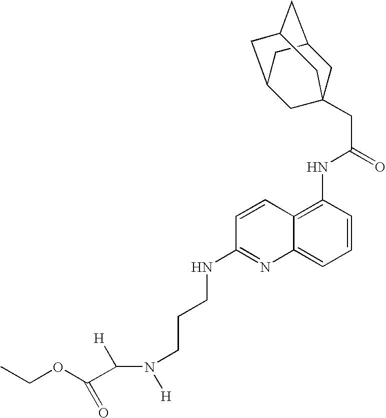 | Ethyl {[3-({5-[(1-adamantylacetyl)-amino]quinolin-2-yl}amino)propyl]-amino}acetate | 478.2944 | 479.374 |
| 117 | 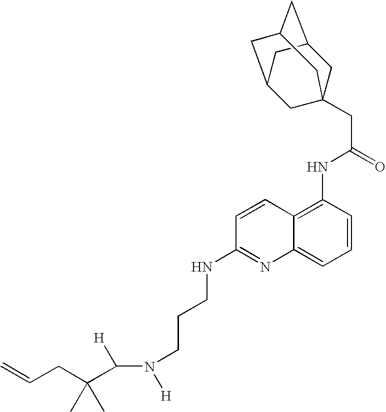 | 2-(1-Adamantyl)-N-[2-({3-[(2,2-dimethylpent-4-enyl)amino]propyl}-amino)quinolin-5-yl]acetamide | 488.3515 | 489.223 |
| 118 | 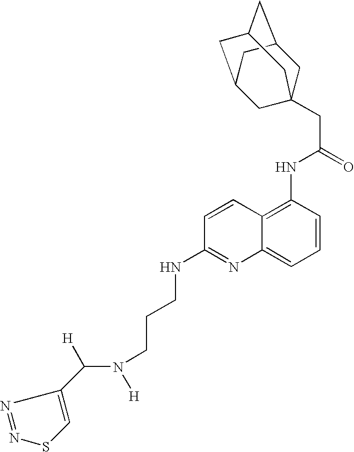 | 2-(1-Adamantyl)-N-[2-({3-[(1,2,3-thiadiazol-4-ylmethyl)amino]-propyl}amino)quinolin-5-yl]acetamide | 490.2515 | 491.316 |

### EXAMPLES 119 TO 157

A series of compound were prepared in a combinatorial chemistry format as follows. 2-(1-Adamantyl)-N-(2-chloroquinolin-5-yl)acetamide (Example 2) (1.42 mg in each well) dissolved in N-methylpyrrolidinone (50 μL in each well) was treated with the amine (8.10⁻⁶ mol in each well), then potassium carbonate (8.10⁶ mol in each well) and potassium iodide (catalytic amount). The reaction mixture was heated up to 120° C. for 36 hours then a further two equivalent of amine were added, reaction heated at 120° C. for 48 hours. A further addition of amine (8.10⁻⁶ mol in each well) and heated up to 120° C. for 72 hours. Each well's content was dissolved in dimethyl sulfoxide (200 μL), shaken, filtered over a porvair box, and the collected solid was washed with dimethyl sulfoxide (200 μL). The filtered content was purified by mass directed purification.

| | | | Theoretical | (M + H) |
|---|---|---|---|---|
| Example No./Structure | | Name | mol. wt. | collected |
| 119 | 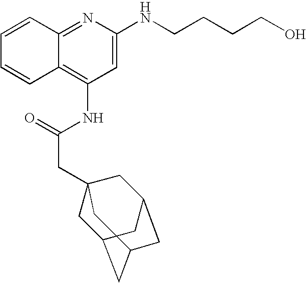 | 2-(1-Adamantyl)-N-{2-[(4-hydroxybutyl)-amino]quinolin-5-yl}acetamide | 407.2573 | 408.295 |
| 120 | 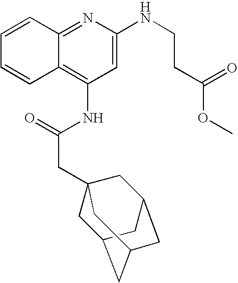 | Methyl 3-({5-[(1-adamantylacetyl)-amino]quinolin-2-yl}amino)propanoate | 421.2365 | 422.297 |
| 121 | 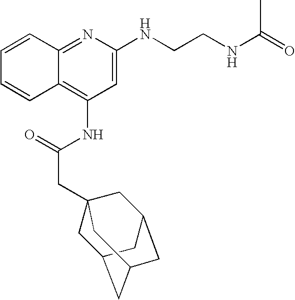 | N-(2-{[2-(Acetylamino)ethyl]-amino}quinolin-5-yl)-2-(1-adamantyl)-acetamide | 420.2525 | 421.321 |
| 122 | 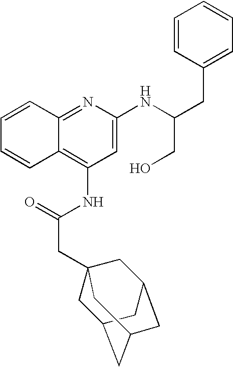 | 2-(1-Adamantyl)-N-{2-[(1-benzyl-2-hydroxyethyl)amino]-quinolin-5-yl}acetamide | 469.2729 | 470.329 |
| 123 | 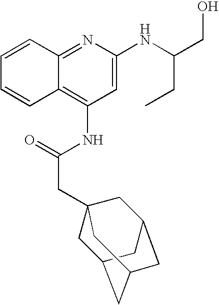 | 2-(1-Adamantyl)-N-(2-{[1-(hydroxymethyl)-propyl]amino}quinolin-5-yl)acetamide | 407.2573 | 408.349 |
| 124 | 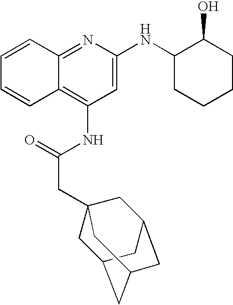 | 2-(1-Adamantyl)-N-(2-{[(2S)-2-hydroxycyclohexyl]-amino}quinolin-5-yl)acetamide | 433.2729 | 434.348 |
| 125 | 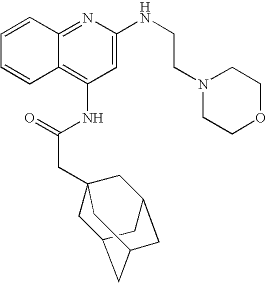 | 2-(1-Adamantyl)-N-{2-[(2-morpholin-4-ylethyl)amino]-quinolin-5-yl}acetamide | 448.2838 | 449.21 |
| 126 | 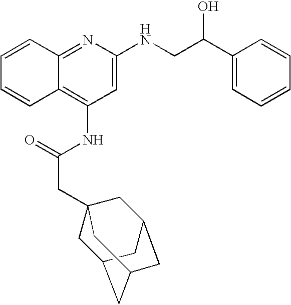 | 2-(1-Adamantyl)-N-{2-[(2-hydroxy-2-phenylethyl)amino]-quinolin-5-yl}acetamide | 455.2573 | 456.31 |
| 127 | 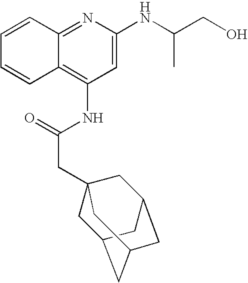 | 2-(1-Adamantyl)-N-{2-[(2-hydroxy-1-methylethyl)amino]-quinolin-5-yl}acetamide | 393.2416 | 394.324 |
| 128 | 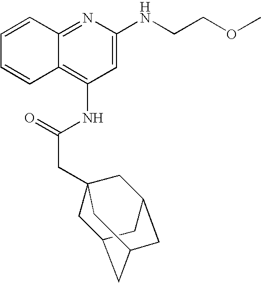 | 2-(1-Adamantyl)-N-{2-[(2-methoxyethyl)-amino]quinolin-5-yl}acetamide | 393.2416 | 394.331 |
| 129 | 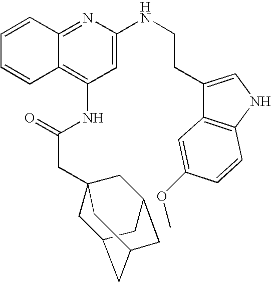 | 2-(1-Adamantyl)-N-(2-{[2-(5-methoxy-1H-indol-3-yl)ethyl}-amino}quinolin-5-yl)acetamide | 508.2838 | 509.32 |
| 130 | 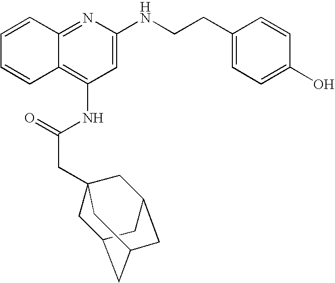 | 2-(1-Adamantyl)-N-(2-{[2-(4-hydroxyphenyl)-ethyl]amino}quinolin-5-yl)acetamide | 455.2573 | 456.348 |
| 131 | 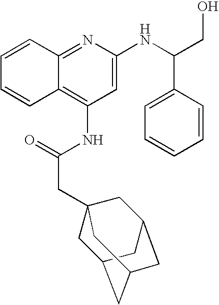 | 2-(1-Adamantyl)-N-{2-[(2-hydroxy-1-phenylethyl)amino]-quinolin-5-yl}acetamide | 455.2573 | 456.279 |
| 132 | 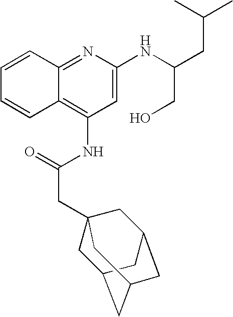 | 2-(1-Adamantyl)-N-(2-{[1-(hydroxymethyl)-3-methylbutyl]amino}-quinolin-5-yl)-acetamide | 435.2885 | 436.371 |
| 133 | 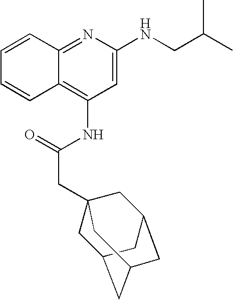 | 2-(1-Adamantyl)-N-[2-(isobutylamino)-quinolin-5-yl]-acetamide | 391.2624 | 392.348 |
| 134 | 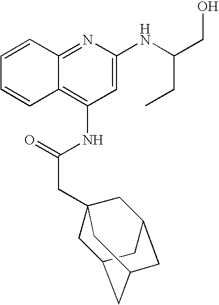 | 2-(1-Adamantyl)-N-(2-{[1-(hydroxymethyl)-propyl]amino}-quinolin-5-yl)-acetamide | 407.2573 | 408.349 |
| 135 | 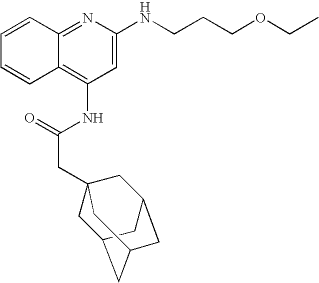 | 2-(1-Adamantyl)-N-{2-[(3-ethoxypropyl)-amino]quinolin-5-yl}acetamide | 421.2729 | 422.258 |
| 136 | 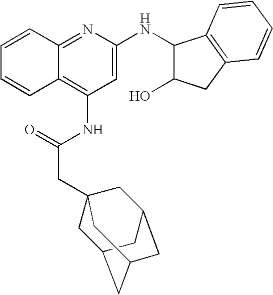 | 2-(1-Adamantyl)-N-{2-[(2-hydroxy-2,3-dihydro-1H-inden-1-yl)amino]quinolin-5-yl}acetamide | 467.2573 | 468.314 |
| 137 | 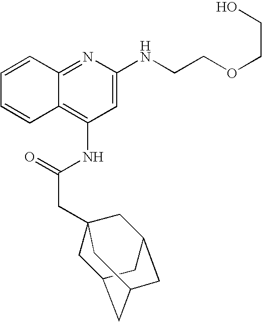 | 2-(1-Adamantyl)-N-(2-{[2-(2-hydroxyethoxy)-ethyl]amino}quinolin-5-yl)acetamide | 423.2522 | 424.328 |
| 138 | 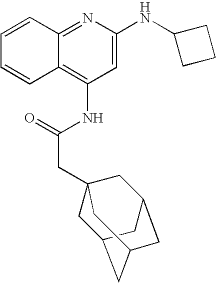 | 2-(1-Adamantyl)-N-[2-(cyclobutylamino)-quinolin-5-yl]-acetamide | 389.2467 | 390.349 |
| 139 | 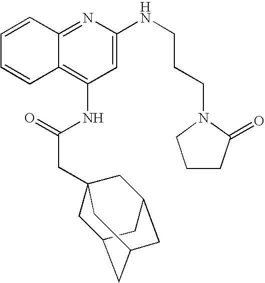 | 2-(1-Adamantyl)-N-(2-{[3-(2-oxopyrrolidin-1-yl)propyl]amino}-quinolin-5-yl)-acetamide | 460.2838 | 461.347 |
| 140 | 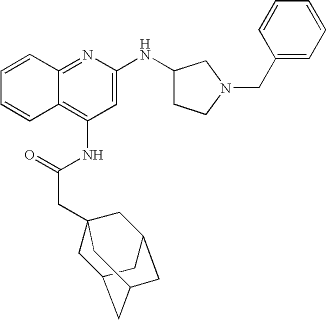 | 2-(1-Adamantyl)-N-{2-[(1-benzylpyrrolidin-3-yl)amino]quinolin-5-yl}acetamide | 494.3045 | 495.237 |
| 141 | 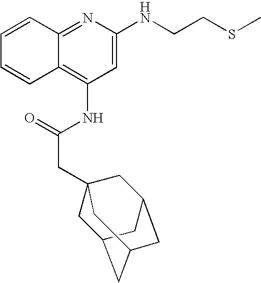 | 2-(1-Adamantyl)-N-(2-{[2-(methylthio)ethyl]-amino}quinolin-5-yl)acetamide | 409.2188 | 410.278 |
| 142 | 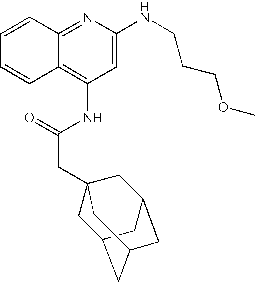 | 2-(1-Adamantyl)-N-{2-[(3-methoxypropyl)-amino]quinolin-5-yl}acetamide | 407.2573 | 408.342 |
| 143 | 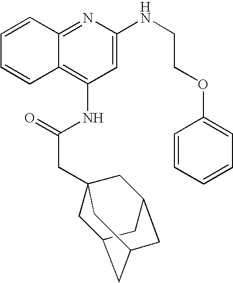 | 2-(1-Adamantyl)-N-{2-[(2-phenoxyethyl)-amino]quinolin-5-yl}acetamide | 455.2573 | 456.31 |
| 144 | 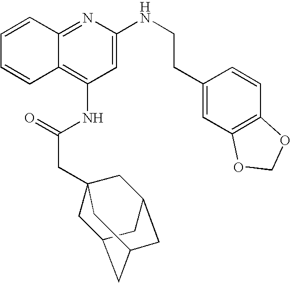 | 2-(1-Adamantyl)-N-(2-{[2-(1,3-benzodioxol-5-yl)ethyl]amino}-qquinolin-5-yl)-acetamide | 483.2522 | 484.302 |
| 145 | 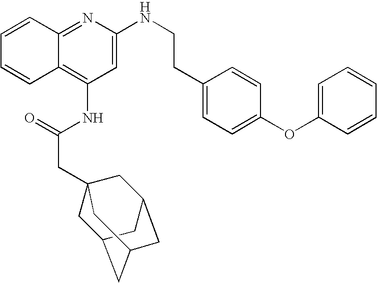 | 2-(1-Adamantyl)-N-(2-{[2-(4-phenoxyphenyl)-ethyl]amino}quinolin-5-yl)acetamide | 531.2886 | 532.339 |
| 146 | 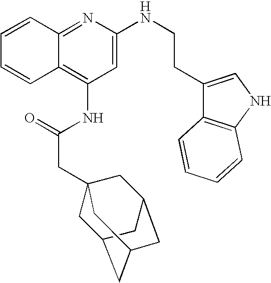 | 2-(1-Adamantyl)-N-(2-{[2-(1H-indol-3-yl)ethyl]amino}-quinolin-5-yl)acetamide | 478.2733 | 479.327 |
| 147 | 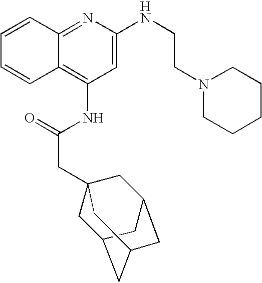 | 2-(1-Adamantyl)-N-{2-[(2-piperidin-1-ylethyl)amino]-quinolin-5-yl}-acetamide | 446.3045 | 447.273 |
| 148 | 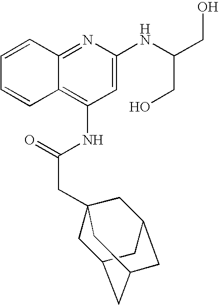 | 2-(1-Adamantyl)-N-(2-{[2-hydroxy-1-(hydroxymethyl)-ethyl]amino}quinolin-5-yl)acetamide | 409.2365 | 410.325 |
| 149 | 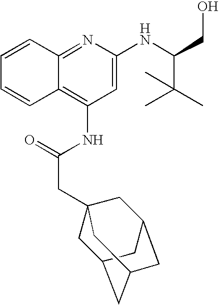 | 2-(1-Adamantyl)-N-(2-{[(1R)-1-(hydroxymethyl)-2,2-dimethylpropyl]-amino}quinolin-5-yl)acetamide | 435.2885 | 436.347 |
| 150 | 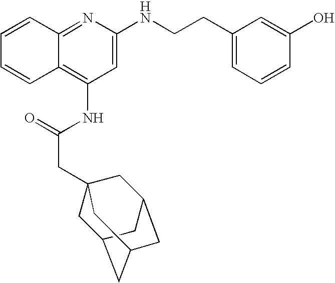 | 2-(1-Adamantyl)-N-(2-{[(2-(3-hydroxyphenyl)-ethyl]-dimethylpropyl]-amino}quinolin-5-yl)acetamide | 455.2573 | 456.31 |
| 151 | 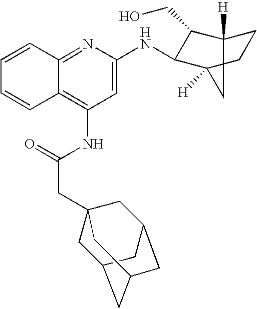 | 2-(1-Adamantyl)-N-(2-{[(1S,3R,4R)-3-(hydroxymethyl)-bicyclo[2.2.1]hept-2-yl]amino}quinolin-5-yl)acetamide | 459.2885 | 460.27 |
| 152 | 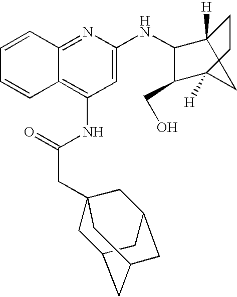 | 2-(1-Adamantyl)-N-(2-{[(1R,3R,4S)-3-(hydroxymethyl)-bicyclo[2.2.1]hept-2-yl]amino}quinolin-5-yl)acetamide | 459.2885 | 460.324 |
| 153 | 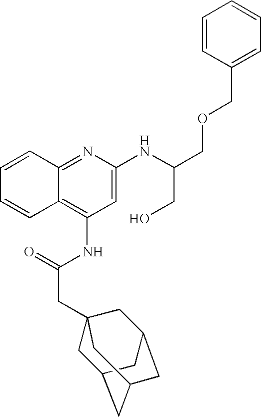 | 2-(1-Adamantyl)-N-(2-{[2-(benzyloxy)-1-(hydroxymethyl)ethyl]-amino}quinolin-5-yl)acetamide | 499.2835 | 500.314 |
| 154 | 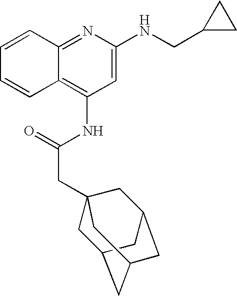 | 2-(1-Adamantyl)-N-{2-[(cyclopropylmethyl)-amino]quinolin-5-yl}acetamide | 389.2467 | 390.333 |
| 155 | 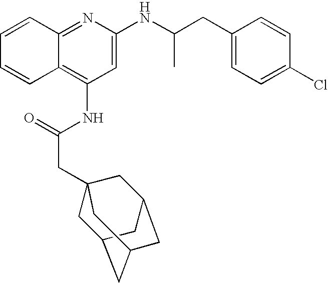 | 2-(1-Adamantyl)-N-(2-{[2-(4-chlorophenyl)-1-methylethyl]amino}-quinolin-5-yl)-acetamide | 487.239 | 488.278 |
| 156 | 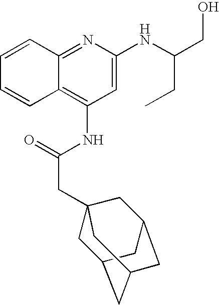 | 2-(1-Adamantyl)-N-(2-{[1-(hydroxymethyl)-propyl]amino}quinolin-5-yl)acetamide | 407.2573 | 408.357 |
| 157 | 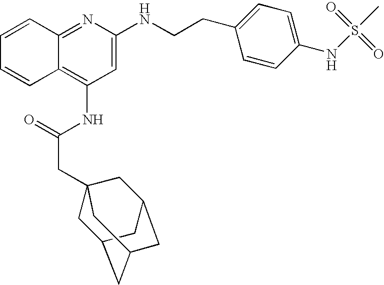 | 2-(1-Adamantyl)-N-{2-[(2-{4-[(methylsulfonyl)-amino]phenyl}ethyl)-amino]quinolin-5-yl}acetamide | 532.2508 | 533.276 |

### EXAMPLE 158

### 2-(1-Adamantyl)-N-[2-({2-[bis(2-hydroxyethyl)amino]ethyl}amino)quinolin-5-yl]acetamide

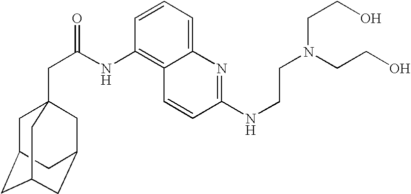

A suspension of 2-(1-adamantyl)-N-{2-[(2-aminoethyl)amino]quinolin-5-yl}acetamide (Example 15) (250 mg) in 1-methyl-2-pyrrolidinone (3 mL) and methanol (5 mL) was treated with glycoaldehyde dimer (86 mg). The mixture was stirred under nitrogen for 5 minutes then 4 drops of acetic acid were added and the solution stirred under nitrogen for a further 30 minutes. Sodium cyanoborohydride (63 mg) was added and the reaction stirred for 2 hours. The reaction was partitioned with water and dichloromethane. The aqueous was further extracted with dichloromethane and the combined organic phases were washed with brine, dried over magnesium sulphate, filtered and evaporated. The residue was purified by column chromatography on silica gel using methanol in dichloromethane at 0% gradually increased to 30% then 7N ammonia in methanol at 30% in dichloromethane. The fractions of interest were combined, concentrated to dryness and the residue, dissolved in minimum amount of dichloromethane was treated with hydrochloric acid at 4M in dioxane. The obtained cloudy solution was fully dissolved in methanol and flushed on SCX column. The column was flushed with methanol then 0.07N ammonia in methanol. The fractions of interest were concentrated to give 26 mg of a cream solid.

¹H NMR (399.978 MHz, CDCl₃) δ 9.73 (s, 1H); 9.51 (s, 1H); 9.35 (s, 1H); 7.72 (d, J=17.6 Hz, 2H); 7.05 (d, J=6.9 Hz, 2H); 6.68 (s, 1H); 3.81 (s, 2H); 3.35 (t, J=4.4 Hz, 4H); 3.17 (s, 2H); 2.91 (s, 4H); 2.70 (s, 1H); 1.65 (s, 2H); 1.39 (s, 3H); 1.19-1.01 (m, 12H).

MS: APCI(+ve) 467/468 (M+1).

### EXAMPLE 159

### 2-(1-Adamantyl)-N-quinolin-5-ylacetamide

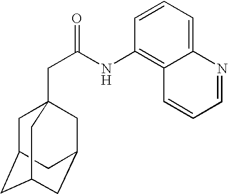

Following the same procedure described in Example 1, 1-adamantylacetic acid (2 g) in dichloromethane (50 mL) and dimethylformamide (50 μL) was reacted with oxalyl chloride (1.01 mL) followed by reaction of the intermediate with 5-aminoquinoline (1.8 g) dissolved in dichloromethane (50 mL) and treatment of the reaction mixture with triethylamine (3 mL) to give 2.38 g of a beige solid.

¹H NMR (400 MHz, DMSO-d₆) δ 9.89 (s, 1H); 8.91 (dd, J=4.1, 1.5 Hz, 1H); 8.47 (dq, J=8.6, 0.8 Hz, 1H); 7.84 (d, J=8.2 Hz, 1H); 7.79 (d, J=6.4 Hz, 1H); 7.73 (t, J=7.8 Hz, 1H); 7.58 (dd, J=8.7, 4.1 Hz, 1H); 2.24 (s, 2H); 1.97 (s, 3H); 1.71 (s, 6H); 1.66 (dd, J=26.8, 12.4 Hz, 6H).

MS: APCI(+ve) 321/322 (M+1).

### EXAMPLE 160

### 2-(1-Adamantyl)-N-isoquinolin-5-ylacetamide

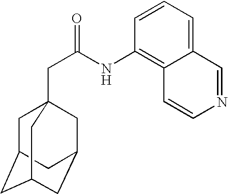

Following the same procedure described in Example 1, 1-adamantylacetic acid (6.94 g) in dichloromethane (70 mL) and dimethylformamide (50 μL) was reacted with oxalyl chloride (4.68 mL) followed by reaction of the intermediate with 5-aminoisoquinoline (5.15 g) dissolved in dichloromethane (50 mL) and treatment of the reaction mixture with triethylamine (10 mL) to give after isolation and purification 8.84 g of a white solid.

¹H NMR (400 MHz, DMSO-d₆) δ 9.87 (s, 1H); 9.31 (s, 1H); 8.55 (d, J=5.9 Hz, 1H); 8.03 (d, J=7.7 Hz, 1H); 7.94 (t, J=8.5 Hz, 2H); 7.66 (t, J=7.8 Hz, 1H); 2.26 (s, 2H); 1.96 (s, 3H); 1.75-1.57 (m, 12H).

MS: APCI(+ve) 321/322 (M+1).

### EXAMPLE 161

### 2-(1-Adamantyl)-N-[2-(3-{[(1R)-2-hydroxy-1-methylethyl]amino}propyl)quinolin-5-yl]acetamide dihydrochloride

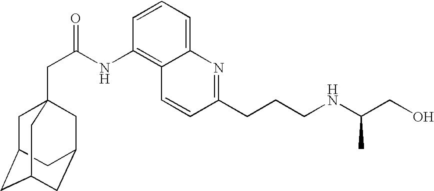

### (i) tert-Butyl 3-{5-[(1-adamantylacetyl)amino]quinolin-2-yl}propyl((1R)-2-{[tert-butyl(dimethyl)silyl]oxy}-1-methylethyl)carbamate

tert-Butyl allyl((1R)-2-{[tert-butyl(dimethyl)silyl]oxy}-1-methylethyl)carbamate (728 mg) was added to a solution of 9-borabicyclo[3.3.1]nonane at 0.5 M in tetrahydrofuran (7 mL) and the solution was heated to reflux for 12 hours. The reaction was cooled to room temperature and a solution of potassium phosphate (1 g) in water (2 mL) was added slowly to the reaction under vigorous stirring condition. A warn solution of 2-(1-adamantyl)-N-(2-chloroquinolin-5-yl)acetamide (Example 2) (500 mg) in dimethylformamide (2 mL) was then added followed by [1,1′-bis(diphenylphosphino)ferrocene]palladium(II)chloride complex (32 mg). The solution was heated to 70° C. for 2 hours, allowed to cool to room temperature then partitioned between ethyl acetate (20 mL) and water (2×20 mL). The aqueous phase was further extracted with ethyl acetate and the combined organics were washed with brine, dried over magnesium sulphate, filtered and evaporated under vacuum. The residue was dissolved in ethyl acetate and washed with water (2×30 mL), then brine (30 mL), dried over magnesium sulphate, filtered and evaporated. The yellow oil was purified by column chromatography on silica eluting with methanol in dichloromethane at 0.5% gradually increased to 2% to give 522 mg of a white solid.

¹H NMR (300 MHz, DMSO-d₆) δ 9.84 (s, 1H); 8.39 (d, J=8.7 Hz, 1H); 7.78-7.71 (m, 2H); 7.68 (t, J=7.7 Hz, 1H); 7.47 (d, J=8.7 Hz, 1H); 3.96-3.79 (m, 1H); 3.61 (dd, J=10.4, 7.3 Hz, 1H); 3.50 (dd, J=10.0, 5.4 Hz, 1H); 3.15 (s, 2H); 2.90 (t, J=7.4 Hz, 2H); 2.25 (s, 2H); 1.97 (s, 4H); 1.72 (s, 6H); 1.67 (dd, J=21.7, 11.9 Hz, 6H); 1.46-1.30 (m, 9H); 1.08 (s, 3H); 0.82 (s, 9H); 0.00 (s, 6H).

MS: APCI(+ve) 650/651 (M+1).

### (ii) 2-(1-Adamantyl)-N-[2-(3-{([(1R)-2-hydroxy-1-methylethyl]amino}propyl)quinolin-5-yl]acetamide dihydrochloride

tert-Butyl 3-{5-[(1-adamantylacetyl)amino]quinolin-2-yl}propyl((1R)-2-{[tert-butyl(dimethyl)silyl]oxy}-1-methylethyl)carbamate (522 mg) was treated with hydrochloric acid at 4M in 1,4-dioxane and stirred for 30 minutes under nitrogen, then evaporated to a yellow foam. The residue was dissolved in minimum amount of methanol at reflux then allowed to cool to room temperature. The clear solution was treated with ethyl acetate until a yellow precipitate developed. The cloudy solution was sonicated, filtered. The resulting solid was dried in a vacuum oven at 40° C. to give 220 mg of the title compound.

¹H NMR (400 MHz, DMSO-d₆) δ 10.30 (s, 1H); 8.91 (d, J=26.7 Hz, 2H); 8.72 (s, 1H); 8.17-7.83 (m, 4H); 3.65 (dd, J=11.7, 4.0 Hz, 1H); 3.51 (dd, J=11.8, 5.4 Hz, 1H); 3.36-3.19 (m, 3H); 3.04 (t, J=6.0 Hz, 2H); 2.30 (s, 2H); 2.28-2.19 (m, 2H); 1.96 (s, 3H); 1.71 (s, 6H); 1.66 (dd, J=28.0, 11.8 Hz, 6H); 1.21 (d, J=6.7 Hz, 3H).

MS: APCI(+ve) 436/437 (M+1).

### EXAMPLE 162

### 2-(1-Adamantyl)-N-(2-{2-[benzyl(2-hydroxyethyl)amino]ethoxy}quinolin-5-yl)acetamide

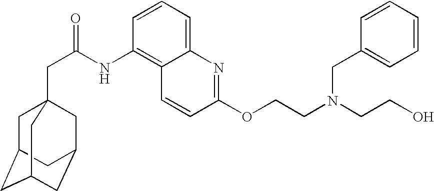

Sodium hydride at 60% in mineral oil (72 mg) was vigorously stirred in hexane (5 mL) for 3 minutes, left to settle for 10 minutes and the solvent was decanted off. The operation was repeated and a solution of 2-[benzyl-(2-hydroxy-ethyl)-amino]-ethanol (330 mg) in 1-methyl-2-pyrrolidinone (2 mL) was added slowly with vigorous stirring for 10 minutes. 2-(1-Adamantyl)-N-(2-chloroquinolin-5-yl)acetamide (Example 2) (300 mg) was added portion wise to give a bright yellow solution. The mixture was subjected to microwave radiation for 15 minutes at 150° C. and 300 W. Ether was added to the black reaction mixture and the precipitate obtained was filtered. Iso-hexane was added to the filtrate, which was left to stand for 5 minutes to allow the formation of a denser brown oil. The supernatant was concentrated under vacuum and the oily residue was purified by column chromatography on silica gel using methanol in dichloromethane from 0% to 5% to afford 234 mg of the title product.

¹H NMR (400 MHz, DMSO-d₆) δ 9.81 (s, 1H); 8.32 (d, J=9.2 Hz, 1H); 7.61-7.53 (m, 3H); 7.35-7.18 (m, 5H); 7.02 (d, J=9.2 Hz, 1H); 4.50 (t, J=6.2 Hz, 2H); 4.37 (t, J=5.4 Hz, 1H); 3.74 (s, 2H); 3.50 (q, J=6.2 Hz, 2H); 2.91 (t, J=6.3 Hz, 2H); 2.64 (t, J=6.4 Hz, 2H); 2.21 (s, 2H); 1.96 (s, 3H); 1.70 (d, J=2.1 Hz, 6H); 1.65 (dd, J=26.6, 12.0 Hz, 6H).

MS: APCI(+ve) 514 (M+1).

### EXAMPLE 163

### 2-(1-Adamantyl)-N-(2-{2-[(2-hydroxyethyl)amino]ethoxy}quinolin-5-yl)acetamide

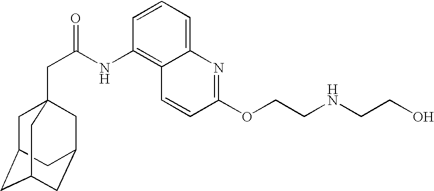

Palladium (10% on charcoal, 20 mg) was moistened with water, diluted with 15 mL of a 20% solution of formic acid in methanol and 2-(1-adamantyl)-N-(2-{2-[benzyl(2-hydroxyethyl)amino]ethoxy}quinolin-5-yl)acetamide (Example 162) (234 mg) was added.

The mixture was submitted to 2.5 relative bars of hydrogen for 3 hours. The suspension was filtered over celite, washed with methanol and the filtrate was concentrated down to an oil. The crude was purified on silica gel. The residue obtained was dissolved in 1:1 mixture of methanol and dichloromethane and treated with hydrochloric acid at 4M in 1,4-dioxane (500 μL). The obtained suspension was filtered and the solid was dissolved in the minimum amount of methanol and treated with ethyl acetate until a solid precipitated out. This solid was collected by filtration and dried in an vacuum oven at 60° C. to give 54 mg of the title compound.

Is ¹H NMR (400 MHz, DMSO-d₆) δ 9.93 (s, 1 μl); 9.05 (s, 2H); 8.40 (d, J=9.2 Hz, 1H); 7.68-7.58 (m, 3H); 7.10 (d, J=9.2 Hz, 1H); 4.80 (s, 1H); 4.71 (t, J=5.3 Hz, 2H); 3.71 (t, J=5.3 Hz, 2H); 3.45 (quintet, J=5.5 Hz, 2H); 3.12 (quintet, J=5.5 Hz, 2H); 2.23 (s, 2H); 1.96 (s, 3H); 1.70 (s, 6H); 1.65 (dd, J=28.0, 11.8 Hz, 6H).

MS: APCI(+ve) 424/425 (M+1)

### EXAMPLE 164

### 2-(1-Adamantyl)-N-{2-[bis(2-hydroxyethyl)amino]quinolin-5-yl}acetamide

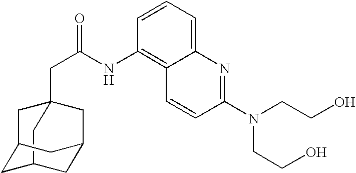

During the purification step in the reaction outlined above (Example 163), there was isolated a second product that was characterized as being the isomer drawn above.

¹H NMR (400 MHz, CD₃OD) δ 8.07 (d, J=9.5 Hz, 1H); 7.53-7.43 (m, 2H); 7.26 (d, J=6.9 Hz, 1H); 7.17-7.07 (m, 1H); 5.43 (d, 1H); 3.84 (d, J=1.8 Hz, 8H); 2.24 (s, 2H); 2.02 (s, 3H); 1.83-1.66 (m, 12H).

MS: APCI(+ve) 424/425 (M+1).

### EXAMPLE 165

### 2-(1-Adamantyl)-N-[8-({2-[(2-hydroxyethyl)amino]ethyl}amino)quinolin-5-yl]acetamide

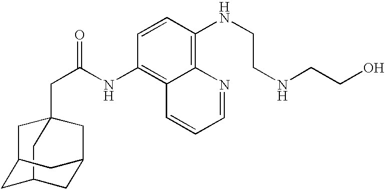

### (i) tert-Butyl 2-[(tert-butoxycarbonyl)(2-hydroxyethyl)amino]ethyl(5-nitroquinolin-8-yl)carbamate

di-(tert-Butyl)dicarbonate (790 mg) was added to a solution of 2-({2-[(5-nitroquinolin-8-yl)amino]ethyl}amino)ethan-1-ol (500 mg) in dichloromethane (20 mL). The solution was stirred for 10 minutes and triethylamine (250 μL) was added. The obtained yellow solution was heated at reflux for 14 hours. A further 2 equivalent of di-(tert-butyl)dicarbonate was added and the solution heated to reflux for 2 hours. 4-Dimethylaminopyridine (220 mg) was added and the reaction was refluxed for 2 hours. The reaction was concentrated under vacuum and purified by flash column chromatography on silica gel eluting with dichloromethane to give 512 mg of the sub-title compound.

¹H NMR (300 MHz, DMSO-d₆) δ 9.30 (d, J=8.3 Hz, 1H); 8.84 (s, 1H); 8.54 (d, J=9.2 Hz, 1H); 8.34 (t, J=5.4 Hz, 1H); 7.83 (t, J=3.9 Hz, 1H); 6.81 (d, J=9.2 Hz, 1H); 4.11 (t, J=4.8 Hz, 2H); 3.61 (q, J=5.7 Hz, 2H); 3.53 (t, J=5.4 Hz, 2H); 1.38 (s, 18H).

MS: APCI(+ve) 477/478 (M+1).

### (ii) tert-Butyl 5-aminoquinolin-8-yl{2-[(tert-butoxycarbonyl)(2-hydroxyethyl)amino]ethyl}carbamate

tert-Butyl 2-[(tert-butoxycarbonyl)(2-hydroxyethyl)amino]ethyl(5-nitroquinolin-8-yl)carbamate (160 mg), iron powder (Example 165 step (i)) (160 mg) and ammonium chloride (160 mg) in a 1:1 mixture of ethanol in water (20 mL) were heated to 60° C. for 1.5 hours under nitrogen. The reaction was allowed to cool to room temperature then filtered over celite, washed with ethanol (20 mL) then ethyl acetate (30 mL). The filtrate was evaporated to give an aqueous residue that was extracted with dichloromethane (20 mL). The aqueous was further extracted with dichloromethane (20 mL) and the combined organics were washed with brine (30 mL), dried over magnesium sulphate, filtered and evaporated in vacuo to give a brown oil. The residue was purified by flash column chromatography on silica gel using a mixture of methanol and dichloromethane from 0% gradually increased to 10%. Yield: 115 mg.

MS: APCI (+ve) 447/448 (M+1).

### (iii) tert-Butyl 5-[(1-adamantylacetyl)amino]quinolin-8-yl{2-[(tert-butoxycarbonyl)(2-hydroxyethyl)amino]ethyl}carbamate

tert-Butyl 5-aminoquinolin-8-yl{2-[(tert-butoxycarbonyl)(2-hydroxyethyl)amino]-ethyl}carbamate (Example 165 step (ii)) (100 mg) in 1-methyl-2-pyrrolidinone (2 mL) was treated with 1-adamantylacetic acid (40 mg) then bromo-tris-pyrrolidino-phosphonium hexafluorophosphate (186 mg) and the solution was stirred for 15 minutes under nitrogen. Triethylamine (56 μL) was added and the reaction was stirred for a further 16 hours at room temperature under nitrogen. The solution was partitioned between water and ethyl acetate. The aqueous phase was further extracted with ethyl acetate and the combined organic phases were washed with water then brine, dried over magnesium sulphate, filtered and evaporated to a dark red-orange oil. The residue was purified by RPHPLC (0.2% aqueous 7N methanolic ammonia and acetonitrile from 45% organic to 95%) to give 83 mg of the sub-title compound as a yellow solid.

¹H NMR (400 MHz, DMSO-d₆, 90° C.) δ 9.22 (s, 1H); 8.70 (dd, J=4.1, 1.8 Hz, 1H); 8.22 (dd, J=8.6, 1.7 Hz, 1H); 7.50 (dd, J=8.5, 4.1 Hz, 1H); 7.34 (d, J=8.2 Hz, 1H); 6.68 (d, J=8.2 Hz, 1H); 6.48 (t, J=5.9 Hz, 1H); 4.12 (t, J=5.8 Hz, 2H); 3.51 (t, J=6.2 Hz, 2H); 3.48-3.40 (m, 4H); 2.15 (s, 2H); 1.96 (s, 3H); 1.72 (s, 6H); 1.67 (dd, J=26.0, 11.5 Hz, 6H); 1.41 (s, 9H); 1.39 (s, 9H).

MS: APCI(+ve) 623/624 (M+1).

### (iv) 2-(1-Adamantyl)-N-[8-{2-[(2-hydroxyethyl)amino]ethyl}amino)quinolin-5-yl]acetamide trihydrochloride

tert-butyl 5-[(1-adamantylacetyl)amino]quinolin-8-yl{2-[(tert-butoxycarbonyl)(2-hydroxyethyl)amino]ethyl}carbamate (Example 165 step (iii)) (55 mg) in chloroform (12 mL) was treated with hydrochloric acid at 4M in dioxan and stirred overnight under nitrogen at room temperature. The resulting orange suspension was sonicated and filtered to leave a red solid that was washed with ether, dried in a vacuum oven at 40° C. to afford 30 mg of the title compound as an orange solid.

¹H NMR (400 MHz, DMSO-d₆) δ9.63 (s, 1H); 8.96 (s, 2H); 8.82 (dd, J=4.2, 1.7 Hz, 1H); 8.34 (dd, J=8.5, 1.5 Hz, 1H); 7.63 (dd, J=8.6, 4.2 Hz, 1H); 7.41 (dd, J=−8.2, 3.6 Hz, 1H); 6.86 (d, J=8.2 Hz, 1H); 3.70 (d, J=5.1 Hz, 2H); 3.67 (d, J=7.4 Hz, 2H); 3.23 (quintet, J=5.3 Hz, 2H); 3.06 (quintet, J=5.0 Hz, 2H); 2.17 (s, 2H); 1.97 (s, 3H); 1.75-1.58 (m, 12H).

MS: APCI(+ve) 423/424 (M+1).

### EXAMPLE 166

### 2-(1-Adamantyl)-N-{8-[(2-aminoethyl)thio]quinolin-5-yl}acetamide

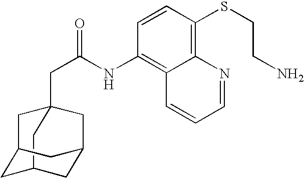

Cysteamine (87 mg) dissolved in 1-methyl-2-pyrrolidinone (2 mL) was treated under vigorous stirring with sodium hydride at 60% in mineral oil (45 mg) in a nitrogen atmosphere and stirred for 16 hours. 2-(1-Adamantyl)-N-{2-chloro-quinolin-5-yl}acetamide (200 mg) was added and the reaction was subjected to 300 W microwave radiation at 150° C. for 15 minutes. The reaction mixture was partitioned between dichloromethane (20 mL), brine (10 mL) and 2M aqueous hydrochloric acid (10 mL). The dichloromethane was further washed with brine (20 mL) and 2M aqueous hydrochloric acid (10 mL). The combined aqueous phases were basified with 2M aqueous sodium hydroxide (30 mL) and extracted with dichloromethane (60 mL). The isolated organic phase was concentrated in vacuo and the residue was partitioned between ethyl acetate (10 mL) and saturated aqueous sodium bicarbonate (10 mL). The ethyl acetate phase was dried over magnesium sulphate, filtered and evaporated to a yellow oil, which was purified by flash column chromatography on silica gel and eluted with a solvent mixture of methanol in dichloromethane at 0% gradually increased to 5% to give 32 mg of a beige solid.

¹H NMR (400 MHz, DMSO-d₆) δ 9.85 (s, 1H); 8.23 (d, J=9.0 Hz, 1H); 7.66 (s, 3H); 7.42 (d, J=9.0 Hz, 1H); 3.32 (t, J=6.8 Hz, 2H); 3.32 (s, 2H); 2.87 (t, J=6.8 Hz, 2H); 2.22 (s, 2H); 1.96 (s, 3H); 1.70-1.60 (m, 12H).

MS: APCI(+ve) 396/397 (M+1).

### EXAMPLE 167

### N-(1-Adamantylmethyl)-6-chloro-2-[3-(methylamino)propyl]quinoline-5-carboxamide sesquihydrochloride

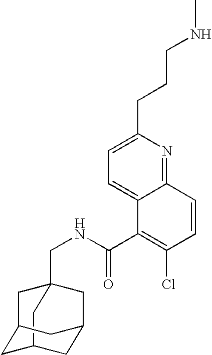

### (i) 2-Chloro-5-{[3-ethoxyprop-2-enoyl]amino}benzoic acid

A solution of 3-ethoxyprop-2-enoyl chloride (1.34 g) in anhydrous tetrahydrofuran (10 mL) was added dropwise to a suspension of 5-amino-2-chlorobenzoic acid (3.79 g) in anhydrous tetrahydrofuran (25 mL). The mixture was heated at 40° C. for 6 hours, diluted with ethyl acetate (25 mL) and washed with 2M aqueous hydrochloric acid solution (25 mL). The organic phase was dried over anhydrous sodium sulphate, filtered and concentrated to give the sub-titled compound (2.5 g) as a yellow oil.

MS: APCI (+ve) 270/272 (M+1)

### (ii) N-(1-Adamantylmethyl)-2,6-dichloroquinoline-5-carboxamide

A mixture of 2-chloro-5-{[3-ethoxyprop-2-enoyl]amino}benzoic acid (Example 167 (i)) (2.5 g) and concentrated sulphuric acid (25 mL) was heated at 60° C. for 3 hours. The mixture was cooled to room temperature, poured on to ice/water (200 mL) and filtered. The pH of the filtrate was adjusted to 4 by the addition of potassium hydroxide. The resultant precipitate was removed by filtration and the filtrate was neutralised by the addition of 2M aqueous hydrochloric acid. This solution was concentrated and the residue dried by repeated azeotropic removal of water using a 1:1 toluene/acetonitrile mixture. The residue was suspended in phosphoryl chloride (50 mL) and the mixture heated at reflux for 3 hours. The reaction mixture was concentrated and the residue was suspended in dichloromethane (50 mL) and filtered. The filtrate was treated dropwise with a solution of 1-adamantylmethylamine (1.53 g) and triethylamine (2.6 mL) in dichloromethane (10 mL) and stirred for 1 hour. The reaction mixture was washed with water (25 mL), dried over anhydrous sodium sulphate, filtered and concentrated. The residue was triturated with diethyl ether and filtered. The filtrate was concentrated and the residue was purified by chromatography on silica gel eluting with iso-hexane:ethyl acetate (6:1 to 3:1) to give the sub-title compound (0.099 g).

MS: APCI (+ve) 389/391 (M+1).

### (iii) N-(1-Adamantylmethyl)-6-chloro-2-[3-(methylamino)propyl]quinoline-5-carboxamide sesquihydrochloride

A solution of tert-butyl allyl(methyl)carbamate (0.050 g) in 9-boroabicyclo[3.3.1]nonane (1.14 mL of a 0.5M solution in tetrahydrofuran) was heated at reflux under nitrogen for 4 hours. The solution was cooled to room temperature and potassium phosphate (0.28 mL of a 3M solution in water) was added. The mixture was stirred for 15 minutes and a solution of N-(1-adamantylmethyl)-2,6-dichloroquinoline-5-carboxamide (Example 167 (ii)) (0.093 g) and tetrakis(triphenylphosphine)palladium (II) (0.005 g) in anhydrous N,N-dimethylformamide (3 mL) was added. The mixture was heated at 60° C. for 3 hours, diluted with saturated brine (25 ml) and extracted into ethyl acetate (3×25 ml). The combined extracts were dried over anhydrous sodium sulphate, filtered and concentrated. The residue was purified by chromatography on silica gel eluting with iso-hexane:ethyl acetate (9:1 to 1:1). The isolated material was dissolved in a solution of hydrogen chloride in dioxane (10 ml of a 4M solution) and concentrated; the resultant solid was recrystallised from ethyl acetate/methanol and the solid collected by filtration to afford the title compound (0.052 g) as a colourless powder.

¹H NMR (400 MHz, DMSO-d₆) δ 8.89 (2H, broad); 8.67 (1H, t); 8.15 (1H, d); 8.07 (1H, d); 7.84 (1H, d); 7.66 (1H, d); 3.10-2.90 (6H, m); 2.15 (2H, quintet); 1.97 (3H, s); 1.75-1.55 (12H, m).

MS: APCI (+ve) 426/428 (M+1).

MP: 184-185° C.

### EXAMPLE 168

### N-(1-Adamantylmethyl)-2-[3-{(3-hydroxypropyl)amino]propyl}quinoline-4-carboxamide benzoic acid salt

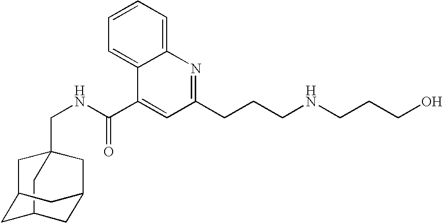

### (i) N-(1-Adamantylmethyl)-2-bromoquinoline-4-carboxamide

2-Hydroxyquinoline-4-carboxylic acid (1.89 g) was added to phosphorus oxybromide (8.61 g) and toluene (10 mL) and the resulting suspension was stirred at 150° C. for 3 hours, cooled and concentrated. The residue was suspended in ethyl acetate (100 mL) and cooled to 5° C. A solution of 1-adamantylmethylamine (1.65 g) and triethylamine (2.52 g) in ethyl acetate (20 mL) was added dropwise maintaining the temperature of the reaction below 10° C. After complete addition the mixture was stirred for 2 hours, poured into 1N hydrochloric acid and was extracted into ethyl acetate (2×50 mL). The combined extracts were dried over anhydrous magnesium sulfate, filtered and concentrated. The residue was purified by chromatography on silica gel eluting with ethyl acetate/iso-hexane (1/3) to afford the sub-title compound as a white solid (1.81 g).

¹H NMR (300 MHz, CDCl₃) δ 8.17 (1H, d); 8.06 (1H, d); 7.77 (1H, t); 7.63 (1H, t); 7.55 (1H, s); 6.05 (1H, t); 3.25 (2H, d); 2.04 (3H, s); 1.76 (3H, d); 1.66 (3H, d); 1.59 (6H, s).

MS: APCI(+ve) 400/401 (M+1)

MP: 196-197° C. (dec.)

### (ii) N-(1-Adamantylmethyl)-2-{3-[(3-hydroxypropyl)amino]propyl}quinoline-4-carboxamide benzoic acid salt

A solution of tert-butyl allyl(3-{[tert-butyl(dimethyl)silyl]oxy}propyl)carbamate (0.493 g) in 9-boroabicyclo[3.3.1]nonane (6 ml of a 0.5M solution in tetrahydrofuran) was heated at reflux under nitrogen for 3 hours. The solution was cooled to room temperature and potassium phosphate (2 ml of a 3M solution in water) was added. The mixture was stirred for 15 minutes and a solution of N-(1-adamantylmethyl)-2-bromoquinoline-4-carboxamide (0.400 g) and dichloro[1,1′-bis(diphenylphosphino)ferrocenyl]palladium (II) (0.022 g) in anhydrous N,N-dimethylformamide (3 ml) was added. The mixture was stirred for 6 hours, diluted with saturated brine (25 ml) and extracted into ethyl acetate (3×25 ml). The combined extracts were dried over anhydrous magnesium sulphate, filtered and concentrated. The residue was purified by chromatography on silica gel eluting with iso-hexane:ethyl acetate (4:1 to 2:1). The isolated material was dissolved in a solution of hydrogen chloride in dioxane (10 ml of a 4M solution) and concentrated; the resultant hygroscopic solid was dissolved in water (10 ml), basified with 1N sodium hydroxide solution and extracted into ethyl acetate (3×25 ml). The combined extracts were dried over anhydrous magnesium sulphate, filtered and concentrated. The residue was dissolved in ethyl acetate (10 ml) and benzoic acid (0.25 g) was added. The resulting precipitate was filtered and was re-crystallised from ethyl acetate to afford the title compound as a white solid (0.083 g).

¹H NMR (300 MHz, CDCl₃) δ 8.19 (1H, d); 8.06 (1H, d); 7.97 (2H, dd); 7.71 (1H, dt); 7.56 (1H, dd); 7.46 (1H, t); 7.42 (1H, s); 7.36 (2H, d); 6.91 (1H, t); 3.74 (2H, t); 3.20 (2H, d); 3.12 (2H, t); 3.05 (2H, t); 2.97 (2H, t); 2.31 (2H, m); 2.04 (3H, s); 1.87 (2H, m); 1.76 (3H, d); 1.66 (3H, d); 1.59 (6H, s).

MS: APCI(+ve) 436/437 (M+1)

MP: 147-148° C. (dec.)

### EXAMPLE 169

### N-(1-Adamantylmethyl)-8-[3-(methylamino)propyl]quinoline-4-carboxamide dihydrochloride

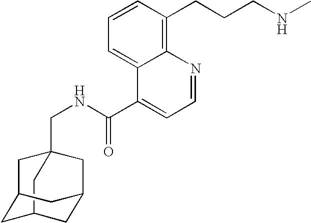

### (i) N-(1-Adamantylmethyl)-8-bromoquinoline-4-carboxamide

To a stirred suspension of 8-bromoquinoline-4-carboxylic acid (2.52 g) in dichloromethane (20 mL) was added oxalyl chloride (1.9 g) and the resulting mixture was stirred for 5 hours and was then concentrated. The residues was suspended in ethyl acetate (100 mL) and cooled to 5° C. A solution of adamantylmethylamine (1.65 g) and triethylamine (3.5 mL) in ethyl acetate (20 mL) was added dropwise maintaining the temperature of the reaction below 10° C. After complete addition the mixture was stirred for 2 hours, poured into 1N hydrochloric acid and the resulting suspension was filtered to afford the sub-title compound as a brown solid (2.30 g).

¹H NMR (400 MHz, DMSO-d₆) δ 9.08 (1H, d); 8.69 (1H, t); 8.20 (1H, dd); 8.10 (1H, dd); is 7.64 (1H, d); 7.58 (1H, dd); 3.06 (2H, d); 1.97 (3H, s); 1.70 (3H, d); 1.66 (3H, d); 1.56 (6H, s).

MS: APCI(+ve) 400/401 (M+1)

MP: 240-242° C. (dec.)

### (ii) N-(1-Adamantylmethyl)-8-[3-(methylamino)propyl]quinoline-4-carboxamide dihydrochloride

By the method outlined in Example 161 step (i), a solution of tert-butyl allyl(methyl)carbamate (0.256 g) in 9-boroabicyclo[3.3.1]nonane (6 ml of a 0.5M solution in tetrahydrofuran) was heated at reflux under nitrogen for 3 hours. The solution was cooled to room temperature and potassium phosphate (2 ml of a 3M solution in water) was added. The mixture was stirred for 15 minutes and a solution of N-(1-adamantylmethyl)-8-bromoquinoline-4-carboxamide (0.399 g) and dichloro[1,1′-bis(diphenylphosphino)ferrocenyl]palladium (II) (0.020 g) in anhydrous N,N-dimethylformamide (3 ml) was added. The mixture was heated to 60° C., stirred for 2 hours, diluted with saturated brine (25 ml) and extracted into ethyl acetate (3×25 ml). The combined extracts were dried over anhydrous magnesium sulphate, filtered and concentrated. The residue was purified by chromatography on silica gel eluting with iso-hexane:ethyl acetate (4:1 to 2:1). The isolated material was dissolved in a solution of hydrogen chloride in dioxane (10 ml of a 4M solution) and concentrated; the resultant solid was re-crystallised from methanol-ethyl acetate to afford the title compound as a white solid (0.183 g).

¹H NMR (400 MHz, DMSO-d₆) δ 9.01 (1H, d); 8.75 (2H, br); 8.65 (1H, t); 7.98 (1H, dd); 7.71 (1H, dd); 7.62 (1H, dd); 7.56 (1H, d); 3.29 (2H, t); 3.06 (2H, d); 2.95-2.88 (2H, m); 2.54 (3H, s); 2.08-1.97 (2H, m); 1.97 (3H, s); 1.70 (3H, d); 1.66 (3H, d); 1.56 (6H, s).

MS: APCI(+ve) 392/393 (M+1) N: 243-246° C. (dec.)

### EXAMPLE 170

### N-(1-Adamantylmethyl)-6-chloro-2-(piperazin-1-ylmethyl)quinoline-5-carboxamide hydrochloride

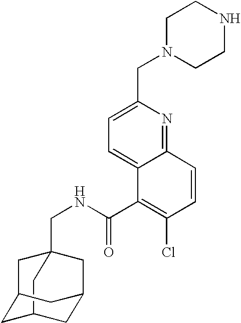

### (i) 6-Chloro-2-methylquinoline-5-carboxylic acid

Crotonaldehyde (1.50 mL) was added dropwise over a period of 1 hour to a mixture of 5-amino-2-chlorobenzoic acid (1.72 g), ferrous sulphate heptahydrate (0.77 g), sodium m-nitrobenzenesulphonate (1.23 g) and concentrated hydrochloric acid (11 mL) at 95° C. The reaction mixture was heated for a further 15 minutes then filtered whilst still hot. The collected solid was extracted with boiling 2M aqueous hydrochloric acid solution (20 mL) and the extract combined with the filtrate. Ammonium acetate was then added to give a solution of pH 4, which was cooled in ice and the resultant precipitate collected by filtration and washed with water. The solid was dried in vacuo to give the sub-title compound (0.5 g) as a brown powder.

MS: APCI(+ve) 222/224 (M+1)

### (ii) N-(1-Adamantylmethyl)-6-chloro-2-methylquinoline-5-carboxamide

Oxalyl chloride (0.30 mL) was added dropwise to a suspension of 6-chloro-2-methylquinoline-5-carboxylic acid (0.50 g) and N,N-dimethylformamide (1 drop) in dichloromethane (15 mL). The reaction mixture was stirred for 1 hour then treated dropwise with a solution of 1-adamantylmethylamine (0.37 g) and triethylamine (0.63 mL) in dichloromethane (10 mL). The mixture was stirred for 16 hours and washed with saturated aqueous sodium bicarbonate solution (25 mL), 1:1 water:acetic acid (25 mL) and water (25 mL). The organic layer was dried over anhydrous sodium sulphate, filtered and concentrated to give the sub-title compound (0.6 g).

¹H NMR (300 MHz, CD₃OD) δ 8.86 (1H, m); 8.49 (1H, d); 8.09 (1H, d); 8.00 (1H, d); 7.80 (1H, d); 3.22 (2H, d); 2.89 (3H, s); 2.03 (3H, s); 1.90-1.62 (12H, m).

MS: APCI(+ve) 369/371 (M+1)

### (iii) N-(1-Adamantylmethyl)-6-chloro-2-(hydroxymethyl)quinoline-5-carboxamide

A solution of m-chloroperoxybenzoic acid (0.25 g) and N-(1-adamantylmethyl)-6-chloro-2-methylquinoline-5-carboxamide (Example 170 step (ii)) (0.37 g) in dichloromethane (15 mL) was stirred for 1 hour. The solution was washed with saturated aqueous sodium bicarbonate solution (25 mL), dried over anhydrous sodium sulphate, filtered and concentrated. The residue was dissolved in acetic anhydride (7 mL) and the solution was heated at 140° C. for 5 minutes under nitrogen. The solution was concentrated and the residue was suspended in 1:1 methanol:2M aqueous sodium hydroxide solution (20 mL) and stirred for 2 hours. The solution was concentrated and the residue dissolved in ethyl acetate and washed with water (2×25 mL). The organic layer was dried over anhydrous magnesium sulphate, filtered and concentrated to give the sub-title compound (0.41 g).

MS: APCI(+ve) 385/387 (M+1)

### (iv) N-(1-Adamantylmethyl)-6-chloro-2-(piperazin-1-ylmethyl)quinoline-5-carboxamide hydrochloride

A solution of N-(1-adamantylmethyl)-6-chloro-2-(hydroxyethyl)quinoline-5-carboxamide (Example 170 step (iii)) (0.38 g) in dichloromethane (15 mL) was treated in one portion with activated manganese dioxide (0.86 g). The mixture was stirred for 2 hours and filtered through Celite. To the filtrate was added piperazine (0.101 g), powdered 4A molecular sieves (0.20 g), sodium triacetoxyborohydride (0.25 g) and finally acetic acid (0.030 mL). The mixture was stirred for 4 hours, filtered and washed with 2M aqueous sodium hydroxide solution (25 mL) and saturated brine solution (25 mL). The organic layer was dried over anhydrous magnesium sulphate, filtered and concentrated. The residue was purified by chromatography on silica gel eluting with dichloromethane:methanol:concentrated aqueous ammonia (96:4:1). The isolated material was dissolved in a solution of hydrogen chloride in dioxane (10 ml of a 4M solution) and concentrated; the resultant solid was triturated with diethyl ether and the solid collected by filtration to afford the title compound (0.014 g) as a colourless powder.

¹H NMR (400 MHz, DMSO-d₆) δ 9.21 (1H, m); 8.68 (1H, t); 8.17 (1H, d); 8.07 (1H, d); 7.85 (1H, d); 7.80 (1H, d); 3.59 (4H, m); 3.29 (4H, m); 3.08 (2H, d); 1.97 (3H, s); 1.80-1.55 (12H, m).

MS: APCI(+ve) 453/455 (M+1)

MP: 208° C. (dec.)

### EXAMPLE 171

### N-(1-Adamantylmethyl)-quinoline-5-carboxamide trifluoroacetate

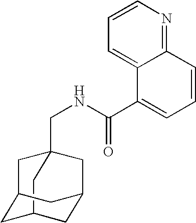

To a stirred suspension of 5-bromoquinoline (0.30 g) in anhydrous diethyl ether (6 mL) was a solution of n-butyl lithium in hexane (2.5M, 0.88 mL) at −78° C. The resulting mixture was stirred for 10 minutes and then a solution of adamantylmethyl isocyanate (0.46 g) in diethyl ether (2 mL) was dropwise added. The reaction was allowed to attain room temperature and was then poured into 1N hydrochloric acid and the resulting mixture extracted into ethyl acetate (3×20 mL). The combined extracts were dried over anhydrous magnesium sulfate, filtered and concentrated. The residue was purified by reverse phase hplc eluting with 0.1M aqueous trifluoroacetic acid in methanol to afford the title compound (0.028 g) as a white solid as its trifluoroacetate salt.

¹H NMR (300 MHz, DMSO-d₆) δ 9.57 (1H, br s); 9.04 (1H, dm); 8.77 (1H, d); 8.59 (1H, t); 8.18 (1H, d); 7.90 (1H, t); 7.82 (1H, t); 7.73 (1H, dd), 3.1 (2H, m); 1.97 (3H, m), 1.78-1.50 (12H, m).

MS: APCI(+ve) 321 (M+1)

MP: 125-127° C. (dec.)

### EXAMPLE 172

### N-(1-Adamantylmethyl)-2-{3-[(3-hydroxypropyl)amino]propyl}quinoline-5-carboxamide dihydrochloride

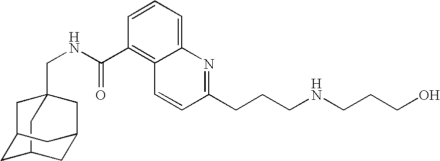

To quinoline-5-carboxylic acid (1.5 g) (prepared in accordance to J. Chem. Soc. 413-417, 1943) in acetic acid was added hydrogen peroxide solution (27% in water). The mixture was warmed to 70° C. and the reaction stirred for 10 hours. The mixture was cooled and evaporated to a give an oil which was then added cautiously to a stirred solution of phosphorus oxychloride (5 mL). The solution was warmed to 60° C., the reaction stirred for 3 hours and then cooled to room temperature. The mixture was evaporated to a concentrated oil under reduced pressure and the crude residue redissolved in dichloromethane (5 mL) and added to a mixture of (1-adamantylmethyl)amine (2.1 g), triethylamine (2.8 mL) in dichloromethane (10 mL). The mixture was stirred for 2 hours at room temperature and then poured into saturated aqueous sodium bicarbonate solution. The organic layer was separated and the aqueous layer further extracted with dichloromethane. The combined extracts were dried over anhydrous magnesium sulphate, filtered and concentrated. The residue was purified by chromatography on silica gel eluting 10% methanol in dichloromethane to afford N-(1-adamantylmethyl)-2-chloroquinoline-5-carboxamide.

A solution of tert-butyl allyl(3-{[tert-butyl(dimethyl)silyl]oxy}propyl)carbamate (0.30 g) in 9-boroabicyclo[3.3.1]nonane (4 ml of a 0.5M solution in tetrahydrofuran) was heated at reflux under nitrogen for 3 hours. The solution was cooled to room temperature and potassium phosphate (2 ml of a 3M solution in water) was added. The mixture was stirred for 15 minutes and a solution of N-(1-adamantylmethyl)-2-chloroquinoline-5-carboxamide (0.300 g) and tetrakistriphenylphosphine palladium (0) (0.020 g) in anhydrous N,N-dimethylformamide (3 ml) was added. The mixture was heated to 60° C. stirred for 2 hours, diluted with saturated brine (25 ml) and extracted into ethyl acetate (3×25 ml). The combined extracts were dried over anhydrous magnesium sulphate, filtered and concentrated. The residue was purified by chromatography on silica gel eluting 5% methanol in dichloromethane. The isolated material was dissolved in a solution of hydrogen chloride in dioxane (10 ml of a 4M solution) and concentrated; the resultant solid was re-crystallised from methanol-ethyl acetate to afford the title compound as a white solid (0.50 g).

¹H NMR (400 MHz, DMSO-d₆) δ 8.85 (2H, m); 8.74 (1H, d); 8.30 (1H, s); 8.19 (1H, d); 7.85 (1H, t); 7.77 (1H, d); 7.65 (1H, d); 3.51 (2H, d); 3.16 (2H, t); 3.08 (2H, d); 3.05-2.95 (4H, m); 2.22 (2H, m), 1.97 (3H, m); 1.81 (2H, m); 1.75-1.55 (14H, m).

MS: APCI(+ve) 436 (M+1)

MP: 150-152° C.

### EXAMPLE 173

### N-(1-Adamantylmethyl)-2-[3-(ethylamino)propyl]quinoline-5-carboxamide dihydrochloride

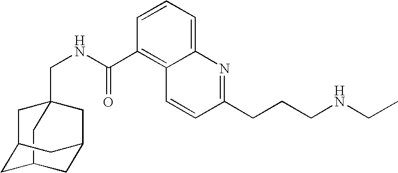

By the method outlined in Example 172, a solution of tert-butyl allyl(ethyl)carbamate (0.185 g) in 9-boroabicyclo[3.3.1]nonane (4 ml of a 0.5M solution in tetrahydrofuran) was heated at reflux under nitrogen for 3 hours. The solution was cooled to room temperature and potassium phosphate (2 ml of a 3M solution in water) was added. The mixture was stirred for 15 minutes and a solution of N-(1-adamantylmethyl)-2-chloroquinoline-5-carboxamide (0.300 g) and tetrakistriphenylphosphine palladium(0) (0.020 g) in anhydrous N,N-dimethylformamide (3 ml) was added. The mixture was heated to 60° C. stirred for 2 hours, diluted with saturated brine (25 ml) and extracted into ethyl acetate (3×25 ml). The combined extracts were dried over anhydrous magnesium sulphate, filtered and concentrated. The residue was purified by chromatography on silica gel eluting 5% methanol in dichloromethane. The isolated material was dissolved in a solution of hydrogen chloride in dioxane (10 ml of a 4M solution) and concentrated; the resultant solid was re-crystallised from methanol-ethyl acetate to afford the title compound as a white solid (0.40 g).

¹H NMR (400 MHz, DMSO-d₆) δ 8.87 (2H, m); 8.72 (1H, d); 8.29 (1H, s); 8.17 (1H, d); 7.84 (1H, t); 7.76 (1H, d); 7.64 (1H, d); 3.16 (2H, t); 3.07 (2H, d); 3.05-2.95 (4H, m); 2.20 (2H, quintet), 1.97 (3H, m); 1.75-1.55 (12H, m); 1.23 (3H, t).

MS: APCI(+ve) 406 (M+1)

MP: 150-155° C.

### EXAMPLE 174

### 2-(1-Adamantyl)-N-[2-({2-[(2-hydroxyethyl)amino]ethyl}amino)-6-methylquinolin-5-yl]acetamide dihydrochloride

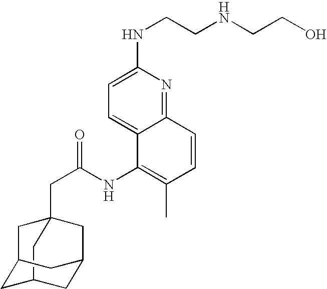

### (i) tert-Butyl[2-({5-[(1-adamantylacetyl)amino]-6-methylquinolin-2-yl}amino)ethyl](2-hydroxyethyl)carbamate

To 2-(1-adamantyl)-N-(2-chloro-6-methylquinolin-5-yl)acetamide (Example 4) (360 mg) and potassium carbonate (270 mg) in N-methylpyrrolidinone (4 mL) was added 2-[(2-aminoethyl)amino]ethanol (500 mg). The mixture was heated to 140° C. and stirred 18 hours under nitrogen. The mixture was cooled to room temperature and poured in water (10 mL). The resulting solution was extracted with dichloromethane (3×10 mL) and the combined organic extracts dried over anhydrous magnesium sulfate, filtered and concentrated. The residue was re-dissolved in dichloromethane (5 mL) and di-tert-butyldicarbonate (150 mg) added. The mixture was stirred for 1 hour and poured into water. The resulting mixture was extracted with dichloromethane (3×10 mL) and the combined organic extracts dried over anhydrous magnesium sulfate, filtered and concentrated. The residue was purified by chromatography on silica gel eluting with 7M NH₃ in methanol:dichloromethane (1:10) to afford the sub-title compound (230 mg) as a colourless oil.

¹H NMR (300 MHz, CD₃OD) δ 7.88 (1H, d); 7.54 (1H, d); 7.41 (1H, d); 6.75 (1H, d); 3.75-3.62 (4H, m); 3.61-3.52 (2H, m); 3.44-3.30 (2H, m); 2.34 (3H, s); 2.29 (2H, s); 2.04 (3H, s); 1.88-1.68 (12H, m); 1.39 (9H, s).

MS: APCI(+ve) 536.8 (M+1)

### (ii) 2-(1-Adamantyl)-N-[2-({2-[(2-hydroxyethyl)amino]ethyl}-amino)-6-methylquinolin-5-yl]acetamide dihydrochloride

tert-Butyl[2-({5-[(1-adamantylacetyl)amino]-6-methylquinolin-2-yl}amino)ethyl](2-hydroxyethyl)carbamate (Example 174 step (i)) (80 mg) was dissolved in dichloromethane (2 mL) and hydrogen chloride in dioxane (10 mL of a 4M solution) was added. The resulting mixture was stirred for 4 hours and then evaporated to dryness. The crude solid was recrystallised from methanol/ethyl acetate. Filtration and drying under vacuum at 40° C. yielded the title compound as a white solid (148 mg).

¹H NMR (300 MHz, DMSO-d₆) δ 9.63 (1H, s); 9.42-8.90 (2H, m); 8.10 (1H, d); 8.00 (1H, d); 7.60 (1H, d); 7.17 (1H, d); 4.04 (2H, t); 3.74 (2H, m), 3.82 (2H, t); 3.11 (2H, m); 2.30 (3H, s); 2.24 (2H, s); 1.97 (2H, s); 1.77-1.60 (12H, m).

MS: APCI(+ve) 437 (M+1)

MP: 227-230° C.

### EXAMPLE 175

### 2-(1-Adamantyl)-N-[2-({2-[(2-hydroxyethyl)amino]ethyl}amino)-6-chloroquinolin-5-yl]acetamide dihydrochloride

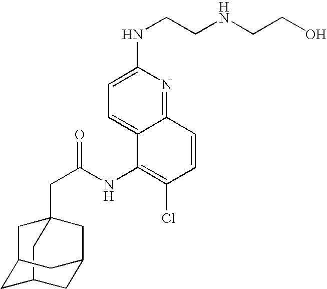

### (i) 2,6-Dichloroquinolin-5-amine

6-Chloro-5-nitroquinoline 1-oxide (4 g) was added to phosphorus oxychloride (15 mL) at 0° C. The solution was allowed to warm to room temperature and stirred for 12 hours. The excess phosphorus oxychloride was evaporated in vacuo and the residue dissolved in water (100 mL)/dichloromethane (100 mL). The layers were separated and the aqueous layer extracted with dichloromethane (2×50 mL). The combined extracts were dried over anhydrous magnesium sulfate, filtered and concentrated to give a brown oil. The residue was dissolved in ethanol/water (1:1, 80 mL), ammonium chloride (2.8 g) and iron (2.8 g) added. The mixture was stirred at 65° C. for 4 hours, cooled to room temperature and filtered. The resulting solid was suspended in dimethylsulphoxide (50 mL), methanol (50 mL) and aqueous hydrochloric acid added (2M, 100 mL). The resulting solid was removed by filtration and then treated with ether (50 mL) and isohexane (50 mL). Evaporation of the mixture afforded the title compound as a solid (1 g).

¹H NMR (400 MHz, DMSO-d₆) δ 8.73 (1H, dd,); 7.62 (1H, d); 7.51 (1H, d); 7.13 (1H, dd); 6.36 (2H, s).

MS: APCI(+ve) 213.1/214.9 (M+1)

### (ii) 2-(1-Adamantyl)-N-(2,6-dichloroquinolin-5-yl)acetamide

To a stirred solution of 2,6-dichloroquinolin-5-amine (Example 175 step (i)) (0.8 g) in N-methylpyrrolidinone (5 ml) was added 4-N,N-dimethylamino pyridine (0.927 g), 1-adamantylacetic acid (1.1 g) and PyBroP (3.5 g). The reaction mixture was heated to 10° C. for 24 hours. The mixture was cooled to room temperature and poured in water (10 mL). The resulting solution was extracted with dichloromethane (3×10 mL) and the combined organic extracts dried over anhydrous magnesium sulfate, filtered and concentrated. The residue was purified by chromatography on silica gel eluting with methanol:dichloromethane (1:10) to afford the sub-title compound (500 mg) as a white solid.

MS: APCI(+ve) 389 (M+1)

### (iii) 2-(1-Adamantyl)-N-[2-({2-[(2-hydroxyethyl)amino]ethyl}amino)-6-chloroquinolin-5-yl]acetamide dihydrochloride

A solution of 2-(1-adamantyl)-N-(2,6-dichloroquinolin-5-yl)acetamide (Example 175 step (ii)) (150 mg) and potassium carbonate (270 mg) in N-methylpyrrolidinone (4 mL) was added 2-[(2-aminoethyl)amino]ethanol (500 mg). The reaction mixture was worked up and the resulting product purified as described in Example 174 to afford the title compound (0.060 g) as an off-white solid.

¹H NMR (300 MHz, DMSO-d₆) δ 9.59 (1H, s); 7.97 (1H, d); 7.90 (1H, d); 7.70 (1H, d); 7.09 (1H, d); 3.93 (2H, m); 3.73 (2H, t); 3.68 (1H, m); 3.54-3.47 (1H, m); 3.30 (2H, t); 3.10 (2H, t); 2.23 (2H, s); 1.97 (3H, m); 1.80-1.58 (12H, m).

MS: APCI(+ve) 458 (M+1)

MP: 219-223° C.

### EXAMPLE 176

### N-(1-Adamantylmethyl)-2-({2-[(2-hydroxyethyl)amino]ethyl}amino)quinoline-5-carboxamide dihydrochloride

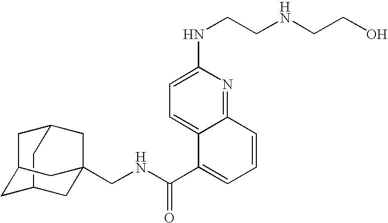

### (i) N-(1-Adamantylmethyl)-2-chloroquinoline-5-carboxamide

A mixture of quinoline-5-carboxylic acid (1.5 g) and acetic acid was treated with hydrogen peroxide solution (40 Vol, 2 mL) and the mixture was heated to 70° C. for 10 hours. The mixture was cooled and concentrated. The residue was added to phosphorus oxychloride (5 mL) and was then heated to 60° C. for 3 hours. The reaction was cooled and concentrated and the residue was dissolved into dichloromethane (20 mL) and a solution of 1-adamantylmethylamine (1 g) and triethylamine (3 ml) in dichloromethane (10 mL) added drop-wise maintaining the temperature below 5° C. The mixture was concentrated and the residue partitioned between ethyl acetate (10 mL) and aqueous hydrochloric acid (1N, 25 mL). The mixture was rapidly stirred for 10 minutes, the product filtered off and vacuum dried to afford the sub-title product as a white solid (0.75 g).

MS: APCI(+ve) 355 (M+1)

### (iii) N-(1-Adamantylmethyl)-2-({2-[(2-hydroxyethyl)amino]ethyl}amino)quinoline-5-carboxamide dihydrochloride

A solution of N-(1-adamantylmethyl)-2-chloroquinoline-5-carboxamide (250 mg) and potassium carbonate (270 mg) in N-methylpyrrolidinone (4 mL) was added 2-[(2-aminoethyl)amino]ethanol (500 mg). The reaction mixture was worked up and the resulting product purified as described in Example 174 to afford the title compound (0.060 g) as an off-white solid.

¹H NMR (400 MHz, DMSO-d₆) δ 9.16 (2H, m); 8.46 (1H, d); 8.34-8.14 (2H, m); 7.72 (1H, t); 7.53 (1H, d); 7.16 (1H, d); 4.01 (2H, m); 3.73 (2H, t); 3.32 (2H, t); 3.11 (2H, t); 3.05 (2H, d); 1.96 (3H, m); 1.80-1.50 (12, m).

MS: APCI(+ve) 423 (M+1)

MP: 220-225° C.

### EXAMPLE 177

### 2-(1-Adamantyl)-N-(2-{3-[(3-hydroxypropyl)amino]propyl}quinolin-5-yl)acetamide dihydrochloride

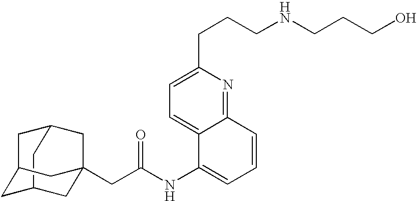

### (i) tert-Butyl 3-{5-[(1-adamantylacetyl)amino]quinolin-2-yl}prop-2-ynyl(3-hydroxypropyl)carbamate

A suspension of 2-(1-adamantyl)-N-(2-chloroquinolin-5-yl)acetamide (Example 2) (0.25 g) tert-butyl 3-hydroxypropyl(prop-2-ynyl)carbamate (0.216 g) in anhydrous acetonitrile (2 ml) and triethylamine (2 ml) was purged with nitrogen for 5 minutes and then copper (I) iodide (0.003 g) and bis-triphenyphosphine palladium dichloride (0.010 g) were added. The mixture was stirred under nitrogen for 2 hours. The mixture was concentrated and the residue was purified by chromatography on silica gel eluting with iso-hexane:ethyl acetate (1:1) to afford the sub-title compound (0.20 g) as a yellow gum.

MS: APCI(+ve) 531.8 (M+1)

### (ii) tert-Butyl 3-{5-[(1-adamantylacetyl)amino]quinolin-2-yl}propyl(3-hydroxypropyl)carbamate

To a stirred solution of tert-butyl 3-{5-[(1-adamantylacetyl)amino]quinolin-2-yl}prop-2-ynyl(3-hydroxypropyl)carbamate (Example 177 step (i)) (0.20 g) in ethanol (8 mL) was added 10% palladium on carbon (0.020 g) and the resulting mixture stirred at room temperature under a 2 bar atmosphere of hydrogen. The mixture was filtered and concentrated to afford the sub-title compound (0.150 g) as an oil.

¹H NMR (300 MHz, DMSO-d₆) δ 9.85 (1H, s); 8.37 (1H, d); 7.81-7.60 (3H, m); 7.48 (1H, d); 4.42 (1H, brs); 3.38 (2H, m); 3.28-3.13 (4H, m); 2.89 (2H, t); 2.23 (2H, s); 1.97 (5H, m); 1.76-1.55 (14H, m); 1.35 (9H, s).

MS: APCI(+ve) 536 (M+1)

### (iii) 2-(1-Adamantyl)-N-(2-{3-[(3-hydroxypropyl)amino]propyl}quinolin-5-yl)acetamide

To a stirred solution of tert-butyl 3-{5-[(1-adamantylacetyl)amino]quinolin-2-yl}propyl(3-hydroxypropyl)carbamate (Example 177 step (ii)) (0.107 g) in 1,4-dioxane (1 mL) was added a solution of anhydrous hydrogen chloride in 1,4-dioxane (4N, 3 mL) and the mixture stirred at room temperature for 2 hours. The mixture was concentrated and the residue triturated with ethyl acetate and filtered to afford the title compound (0.060 g) as a solid.

¹H NMR (400 MHz, DMSO-d₆) δ 9.77 (1H, s); 8.84 (2H, m); 8.64 (1H, d); 7.92 (1H, d); 7.85-7.74 (2H, m); 7.62 (1H, d); 3.52 (3H, m); 3.17 (2H, t); 3.07-2.90 (5H, m); 2.27 (2H, s); 2.26-2.17 (2H, m); 1.97 (3H, m); 1.87-1.77 (2H, m); 1.75-1.60 (12H, m).

MS: APCI(+ve) 436 (M+1)

MP: 130-135° C.

### EXAMPLE 178

### 2-(1-Adamantyl)-N-6-methyl-2-piperazin-1-ylquinolin-5-yl)acetamide dihydrochloride

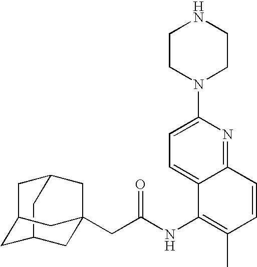

To 2-(1-adamantyl)-N-(2-chloro-6-methylquinolin-5-yl)acetamide (Example 4) (300 mg) and potassium carbonate (600 mg) in N-methylpyrrolidinone (8 mL) was added piperazine (500 mg). The mixture was heated to 140° C. and stirred for 3 hours under nitrogen. The mixture was cooled to room temperature and poured into water (50 mL). The resulting solution was extracted with ethyl acetate (3×50 mL) and the combined organic extracts washed with brine (3×50 mL), dried over anhydrous magnesium sulfate, filtered and concentrated. The residue was purified by chromatography on silica gel eluting with dichloromethane:methanol:aqueous ammonia (19:1:0.1). The resulting oil was dissolved in dichloromethane (5 mL) and a 1M solution of hydrogen chloride in diethyl ether (5 mL) was added. The solid formed was filtered off and dried under vacuum to afford the title compound (0.27 g).

¹H NMR (400 MHz, DMSO-d₆, 90° C.) δ 8.09-8.06 (1H, d); 7.66-7.63 (1H, d); 7.52-7.49 (1H, d); 7.32-7.29 (1H, d); 4.01-3.99 (4H, m); 3.24 (4H, m); 2.29 (3H, s); 2.23 (2H, s); 1.98 (3H, s); 1.75-1.64 (12H, m).

MS: APCI(+ve) 419 (M+1)

MP: 276° C.

### EXAMPLE 179

### 2-(1-Adamantyl)-N-{2-[4-(2-hydroxyethyl)piperazin-1-yl]-6-methylquinolin-5-yl}acetamide dihydrochloride

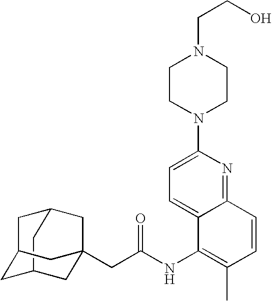

2-(1-Adamantyl)-N-(6-methyl-2-piperazin-1-ylquinolin-5-yl)acetamide (Example 178) (104 mg) and (tert-butyldimethylsilyloxy)acetaldehyde (87 mg) were stirred together in dichloromethane (10 mL). Sodium triacetoxyborohydride (106 mg) was added and the mixture was stirred under nitrogen for 20 hours. The mixture was poured into aqueous sodium bicarbonate solution (50 mL), extracted into dichloromethane (3×50 mL), dried over anhydrous magnesium sulfate, filtered and concentrated. The residue was dissolved in methanol (5 mL) and a solution of anhydrous hydrogen chloride in 1,4-dioxane (4N, 5 mL) added. The mixture was stirred at room temperature for 20 hours. The mixture was concentrated and the residue triturated with ethyl acetate and filtered to afford the title compound (0.046 g) as a solid.

¹H NMR (400 MHz, DMSO-d₆, 90° C.) δ 9.33 (1H, s); 8.07-8.04 (1H, d); 7.59-7.56 (1H, d); 7.49-7.47 (1H, d); 7.32-7.28 (1H, d); 4.00-3.73 (10H, m); 3.25-3.22 (2H, t); 2.28 (3H, s); 2.23 (2H, s); 1.98 (3H, s); 1.76-1.63 (12H, m).

MS: APCI(+ve) 463 (M+1)

MP: 224° C.

### EXAMPLE 180

### 2-(1-Adamantyl)-N-[2-(4-aminopiperidin-1-yl)-6-methylquinolin-5-yl]acetamide dihydrochloride

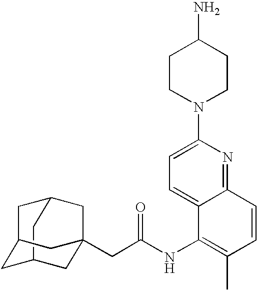

### (i) tert-Butyl 1-{5-[(1-Adamantylacetyl)amino]-6-methylquinolin-2-yl}piperidin-4-ylcarbamate

To 2-(1-adamantyl)-N-(2-chloro-6-methylquinolin-5-yl)acetamide (Example 4) (200 mg) and potassium carbonate (400 mg) in N-methylpyrrolidinone (2 mL) was added tert-butyl piperidin-4-ylcarbanate (1 g). The mixture was heated to 140° C. in a sealed tube and stirred 20 hours. The mixture was cooled to room temperature and poured into water (50 mL). The resulting solution was extracted with ethyl acetate (3×50 mL) and the combined organic extracts washed with brine (3×50 mL), dried over anhydrous magnesium sulfate, filtered and concentrated. The residue was purified by chromatography on silica gel eluting with ethyl acetate to afford the sub-title compound as a gum (0.202 g).

MS: APCI(+ve) 533 (M+1)

### (ii) 2-(1-Adamantyl)-N-[2-(4-aminopiperidin-1-yl)-6-methylquinolin-5-yl]acetamide dihydrochloride

To a stirred solution of tert-butyl 1-{5-[(1-adamantylacetyl)amino]-6-methylquinolin-2-yl}piperidin-4-ylcarbamate (Example 180 step (i)) (0.20 g) in methanol (5 mL) was added a solution of anhydrous hydrogen chloride in 1,4-dioxane (4N, 5 mL) and the mixture stirred at room temperature for 4 hours. The mixture was poured into 2N sodium hydroxide solution (50 mL), extracted with dichloromethane (3×50 mL) and the combined organic extracts dried over anhydrous magnesium sulfate, filtered and concentrated. The residue was purified by chromatography on silica gel eluting with dichloromethane:methanol:aqueous ammonia (9:1:0.1). The resulting oil was dissolved in dichloromethane (5 mL) and a 1M solution of hydrogen chloride in diethyl ether (5 mL) was added. The solid formed was filtered off and dried under vacuum to afford the title compound (0.051 g).

¹H NMR (400 MHz, DMSO-d₆, 90° C.) δ 9.40 (1H, s); 8.08-8.05 (1H, d); 7.53-7.51 (1H, d); 7.37-7.34 (1H, d); 4.55-4.52 (2H, d); 3.53-3.26 (5H, m); 2.28 (3H, s); 2.24 (2H, s); 2.10-2.07 (2H, d); 1.98 (3H, s); 1.75-1.67 (12H, m).

MS: APCI(+ve) 433 (M+1)

MP: 295° C.

### EXAMPLE 181

### 2-(1-Adamantyl)-N-(2-{4-[(2-hydroxyethyl)amino]piperidin-1-yl}-6-methylquinolin-5-yl)acetamide dihydrochloride

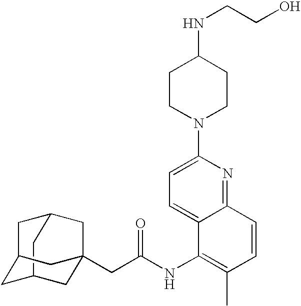

### (i) 2-(1-Adamantyl)-N-(2-{4-[(2-{[tert-butyl(dimethyl)silyl]oxy}ethyl)amino]piperidin-1-yl}-6-methylquinolin-5-yl)acetamide

2-(1-Adamantyl)-N-[2-(4-aminopiperidin-1-yl)-6-methylquinolin-5-yl]acetamide (Example 180) (110 mg) and (tert-butyldimethylsilyloxy)acetaldehyde (42 mg) were stirred together in dichloromethane (10 mL). Sodium triacetoxyborohydride (108 mg) was added and the mixture was stirred under nitrogen for 20 hours. The mixture was poured into aqueous sodium bicarbonate solution (50 mL), extracted into dichloromethane (3×50 mL), dried over anhydrous magnesium sulfate, filtered and concentrated. The residue was purified by chromatography on silica gel eluting with dichloromethane:methanol (9:1) to afford the sub-title compound (0.052 g).

MS: APCI(+ve) 591 (M+1)

### (ii) 2-(1-Adamantyl)-N-(2-{4-[(2-hydroxyethyl)amino]piperidin-1-yl}-6-methylquinolin-5-yl)acetamide dihydrochloride

To a stirred solution of 2-(1-adamantyl)-N-(2-{4-[(2-{[tert-butyl(dimethyl)silyl]oxy}ethyl)amino]piperidin-1-yl}-6-methylquinolin-5-yl)acetamide (Example 181 step (i)) (0.052 g) in methanol (2 mL) was added a solution of anhydrous hydrogen chloride in 1,4-dioxane (4N, 2 mL) and the mixture stirred at room temperature for 4 hours. The mixture was concentrated and the residue triturated with ethyl acetate and filtered to afford the title compound (0.036 g) as a solid.

¹H NMR (400 MHz, DMSO-d₆, 90° C.) δ 8.07-8.05 (1H, d); 7.69 (1H, s); 7.53-7.50 (1H, d); 7.37-7.34 (1H, d); 4.61-4.58 (2H, d); 3.73-3.68 (2H, m); 3.57-3.22 (5H, m); 3.20-3.10 (2H, t); 3.06 (2H, s); 2.28 (2H, s); 2.23-2.18 (3H, d); 1.97 (3H, s); 1.75-1.64 (12H, m).

MS: APCI(+ve) 477 (M+1)

MP: 306° C.

### EXAMPLE 182

### 2-(1-Adamantyl)-N-{2-[(3S)-3-aminopyrrolidin-1-yl]-6-methylquinolin-5-yl}acetamide dihydrochloride

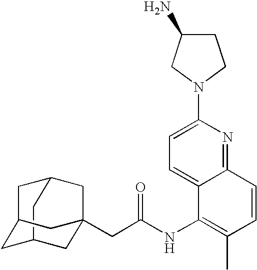

### (i) tert-Butyl(3S)-1-{5-[(1-adamantylacetyl)amino]-6-methylquinolin-2-yl}pyrrolidin-3-ylcarbamate

To 2-(1-adamantyl)-N-(2-chloro-6-methylquinolin-5-yl)acetamide (Example 4) (200 mg) and potassium carbonate (400 mg) in N-methylpyrrolidinone (2 mL) was added tert-butyl (3S)-pyrrolidin-3-ylcarbamate (1 g). The mixture was heated to 140° C. in a sealed tube and stirred 20 hours. The mixture was cooled to room temperature and poured into water (50 mL). The resulting solution was extracted with ethyl acetate (3×50 mL) and the combined organic extracts washed with brine (3×50 mL), dried over anhydrous magnesium sulfate, filtered and concentrated. The residue was purified by chromatography on silica gel eluting with ethyl acetate to afford the sub-title compound as a gum (0.077 g).

MS: APCI(+ve) 519 (M+1)

### (ii) 2-(1-Adamantyl)-N-{2-[(3S)-3-aminopyrrolidin-1-yl]-6-methylquinolin-5-yl}acetamide dihydrochloride

To a stirred solution of tert-butyl (3S)-1-{5-[(1-adamantylacetyl)amino]-6-methylquinolin-2-yl}pyrrolidin-3-ylcarbamate (Example 182 step (i)) (0.077 g) in methanol (5 mL) was added a solution of anhydrous hydrogen chloride in 1,4-dioxane (4N, 5 mL) and the mixture stirred at room temperature for 3 hours. The mixture was poured into 2N sodium hydroxide solution (50 mL), extracted with dichloromethane (3×50 mL) and the combined organic extracts dried over anhydrous magnesium sulfate, filtered and concentrated. The residue was purified by chromatography on silica gel eluting with dichloromethane:methanol:aqueous ammonia (19:1:0.1). The resulting oil was dissolved in dichloromethane (5 mL) and a 1M solution of hydrogen chloride in diethyl ether (5 mL) was added. The solid formed was filtered off, washed with ethyl acetate and dried under vacuum to afford the title compound (0.058 g).

¹H NMR (400 MHz, DMSO-d₆, 90° C.) δ 8.41 (1H, s); 8.15-8.12 (1H, d); 7.87 (1H, s); 7.60-7.58 (1H, d); 7.15-7.13 (1H, d); 4.05-3.81 (5H, m); 3.42-3.26 (2H, m); 2.30 (3H, s); 2.25 (2H, s); 1.98 (3H, s); 1.75-1.58 (12H, m).

MS: APCI(+ve) 419 (M+1)

MP: 291° C.

### EXAMPLE 183

### (3S)—N-((3S)-1-{5-[(1-Adamantylacetyl)amino]-6-methylquinolin-2-yl}pyrrolidin-3-yl)-3-aminopyrrolidine-1-carboxamide dihydrochloride

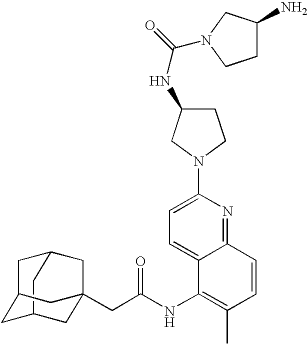

### (i) tert-Butyl (3S)-1-{[((3S)-1-{5-[(1-adamantylacetyl)amino]-6-methylquinolin-2-yl}pyrrolidin-3-yl)amino]carbonyl}pyrrolidin-3-ylcarbamate

To 2-(1-adamantyl)-N-(2-chloro-6-methylquinolin-5-yl)acetamide (Example 4) (200 mg) and potassium carbonate (400 mg) in N-methylpyrrolidinone (2 mL) was added tert-butyl (3S)-pyrrolidin-3-ylcarbamate (1 g). The mixture was heated to 140° C. in a sealed tube and stirred 20 hours. The mixture was cooled to room temperature and poured into water (50 mL). The resulting solution was extracted with ethyl acetate (3×50 mL) and the combined organic extracts washed with brine (3×50 mL), dried over anhydrous magnesium sulfate, filtered and concentrated. The residue was purified by chromatography on silica gel eluting with dichloromethane:methanol (9:1) to afford the sub-title compound as a gum (0.102 g).

MS: APCI(+ve) 631 (M+1)

### (ii) (3S)—N-((3S)-{5-[(1-Adamantylacetyl)amino]-6-methylquinolin-2-yl}pyrrolidin-3-yl)-3-aminopyrrolidine-1-carboxamide dihydrochloride

To a stirred solution of tert-butyl (3S)-1-{[((3S)-1-{5-[(1-adamantylacetyl)amino]-6-methylquinolin-2-yl}pyrrolidin-3-yl)amino]carbonyl}pyrrolidin-3-ylcarbamate (Example 183 step (i)) (102 mg) in methanol (5 mL) was added a solution of anhydrous hydrogen chloride in 1,4-dioxane (4N, 5 mL) and the mixture stirred at room temperature for 3 hours. The mixture was poured into 2N sodium hydroxide solution (50 mL), extracted with dichloromethane (3×50 mL) and the combined organic extracts dried over anhydrous magnesium sulfate, filtered and concentrated. The residue was purified by MCX resin. The resulting oil was dissolved in dichloromethane (5 mL) and a 1M solution of hydrogen chloride in diethyl ether (5 mL) was added. The solvents were removed in vacuo and the solid was washed with ethyl acetate and dried under vacuum to afford the title compound (0.081 g).

¹H NMR (400 MHz, DMSO-d₆, 90° C.) δ 8.25 (1H, br s); 8.15-8.13 (1H, d); 7.97 (1H, br s); 7.64-7.61 (1H, d); 7.21-7.18 (1H, d); 6.35 (1H, br s); 4.42 (1H, br s); 3.97-3.32 (12H, m); 2.30 (3H, s); 2.25 (2H, s); 2.21-2.14 (2H, m); 1.98 (3H, s); 1.74-1.64 (12H, m).

MS: APCI(+ve) 531 (M+1)

MP: 270° C.

### EXAMPLE 184

### 2-(1-Adamantyl)-N-{6-methyl-2-[(1-methylpiperidin-4-yl)amino]quinolin-5-yl}acetamide dihydrochloride

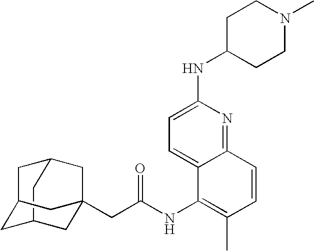

To 2-(1-adamantyl)-N-(2-chloro-6-methylquinolin-5-yl)acetamide (Example 4) (200 mg) and potassium carbonate (400 mg) in N-methylpyrrolidinone (2 mL) was added 1-methylpiperidin-4-amine (1 g). The mixture was heated to 140° C. in a sealed tube and stirred 20 hours. The mixture was cooled to room temperature and poured into brine (50 mL). The resulting solution was extracted with ethyl acetate (3×50 mL) and the combined organic extracts washed with brine (3×50 mL), dried over anhydrous magnesium sulfate, filtered and concentrated. The residue was purified by chromatography on silica gel eluting with dichloromethane:methanol:aqueous ammonia (9:1:0.1), then by reverse-phase hplc using 0.1% aqueous ammonia:acetonitrile (95:5 to 5:95 over 10 mins, Xterra column) followed by treatment with isocyanate resin. The resulting oil was dissolved in dichloromethane (5 mL) and a 1M solution of hydrogen chloride in diethyl ether (5 mL) was added. The solvents were removed in vacuo and the solid was dried under vacuum to afford the title compound (0.027 g).

¹H NMR (400 MHz, CD₃OD) δ 8.13 (1H, br m); 7.83 (1H, br m); 7.66-7.64 (1H, d); 6.99 (1H, br m); 4.28 (1H, br m); 3.60-3.58 (2H, br d); 2.86 (3H, s); 2.31 (5H, m); 2.22 (2H, s); 1.93-1.89 (5H, m); 1.72-1.62 (14H, m).

MS: APCI(+ve) 447 (M+1)

### EXAMPLE 185

### 2-(1-Adamantyl)-N-{6-methyl-2-[(3S)-3-(methylamino)pyrrolidin-1-yl]quinolin-5-yl}acetamide dihydrochloride

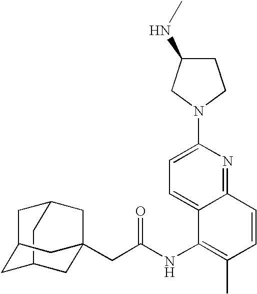

### (i) 2-(1-Adamantyl)-N-{2-[(3R)-3-hydroxypyrrolidin-1-yl]-6-methylquinolin-5-yl}acetamide

To 2-(1-adamantyl)-N-(2-chloro-6-methylquinolin-5-yl)acetamide (Example 4) (950 mg) and potassium carbonate (2 g) in N-methylpyrrolidinone (10 mL) was added (3R)-pyrrolidin-3-ol (3 g). The mixture was heated to 140° C. and stirred 19 hours under nitrogen. The mixture was cooled to room temperature and poured into brine (150 mL). The resulting solution was extracted with ethyl acetate (3×150 mL) and the combined organic extracts washed with brine (3×150 mL), dried over anhydrous magnesium sulfate, filtered and concentrated. The residue was purified by chromatography on silica gel eluting with dichloromethane:methanol (19:1) to afford the sub-title compound as a gum (0.949 g).

MS: ESI(+ve) 420 (M+1)

### (ii) (3R)-1-{5-[(1-adamantylacetyl)amino]-6-methylquinolin-2-yl}pyrrolidin-3-yl methanesulfonate

2-(1-Adamantyl)-N-{2-[(3R)-3-hydroxypyrrolidin-1-yl]-6-methylquinolin-5-yl}acetamide (Example 185 step (i)) (949 mg) and triethylamine (1 mL) in dichloromethane (30 mL) were stirred under nitrogen and cooled to 5° C. Methanesulfonyl chloride (0.5 mL) was added dropwise to this and the mixture was stirred at 5° C. for 30 mins, poured into saturated sodium bicarbonate solution (150 mL), extracted with ethyl acetate (3×150 mL), washed with brine (1×150 mL), dried over anhydrous magnesium sulfate, filtered and concentrated to give the sub-title compound (1.1 g).

MS: ESI(+ve) 498 (M+1)

### (iii) 2-(1-Adamantyl)-N-{6-methyl-2-[(3S)-3-(methylamino)pyrrolidin-1-yl]quinolin-5-yl}acetamide dihydrochloride

To (3R)-1-{5-[(1-adamantylacetyl)amino]-6-methylquinolin-2-yl}pyrrolidin-3-yl methanesulfonate (Example 185 step (ii)) (280 mg) in N-methylpyrrolidinone (5 mL) was added methylamine (40% solution in water, 5 mL). The mixture was heated to 80° C. in a sealed tube and stirred 20 hours. The mixture was cooled to room temperature and poured into 2N sodium hydroxide solution (50 mL). The resulting solution was extracted with ethyl acetate (3×50 mL) and the combined organic extracts washed with brine (3×50 mL), dried over anhydrous sodium sulfate, filtered and concentrated. The residue was purified by chromatography on silica gel eluting with dichloromethane:methanol:aqueous ammonia (19:1:0.1) and then by reverse-phase hplc using 0.1% aqueous trifluoroacetic acid:acetonitrile (95:5 to 50:50 over 10 mins, Xterra column). The resulting oil was dissolved in dichloromethane (5 mL) and a 1M solution of hydrogen chloride in diethyl ether (5 mL) was added. The solvents were removed in vacuo and the solid was dried under vacuum to afford the title compound (0.120 g).

¹H NMR (400 MHz, DMSO-d₆, 90° C.) δ 9.82 (1H, br s); 9.67 (1H, s); 8.22-8.15 (2H, dd); 7.66-7.64 (1H, d); 7.24-7.22 (1H, d); 4.24-4.21 (1H, m); 4.15-4.07 (2H, m); 3.99 (1H, m); 3.90-3.88 (1H, m); 3.42-3.37 (2H, m); 2.66 (3H, s); 2.31 (3H, s); 2.27 (2H, s); 1.98 (3H, br s); 1.75-1.64 (12H, m).

MS: APCI(+ve) 433 (M+1)

### EXAMPLE 186

### 2-(1-Adamantyl)-1-{2-[(3S)-3-(ethylamino)pyrrolidin-1-yl]-6-methylquinolin-5-yl}acetamide dihydrochloride

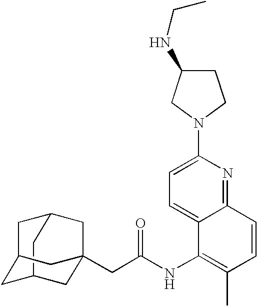

To (3R)-1-{5-[(1-adamantylacetyl)amino]-6-methylquinolin-2-yl}pyrrolidin-3-yl methanesulfonate (Example 185 step (ii)) (280 mg) in N-methylpyrrolidinone (5 mL) was added ethylamine (70% solution in water, 5 mL). The mixture was heated to 80° C. in a sealed tube and stirred 20 hours. The mixture was cooled to room temperature and poured into 2N sodium hydroxide solution (50 mL). The resulting solution was extracted with ethyl acetate (3×50 mL) and the combined organic extracts washed with brine (3×50 mL), dried over anhydrous sodium sulfate, filtered and concentrated. The residue was purified by chromatography on silica gel eluting with dichloromethane:methanol:aqueous ammonia (19:1:0.1) and then by reverse-phase hplc using 0.1% aqueous trifluoroacetic acid:acetonitrile (95:5 to 50:50 over 10 mins, Xterra column). The resulting oil was dissolved in dichloromethane (5 mL) and a 1M solution of hydrogen chloride in diethyl ether (5 mL) was added. The solvents were removed in vacuo and the solid was dried under vacuum to afford the title compound (0.110 g).

¹H NMR (400 MHz, DMSO-d₆, 90° C.) δ 9.80 (1H, br s); 9.66 (1H, s); 8.21-8.14 (2H, dd); 7.66-7.64 (1H, d); 7.23-7.21 (1H, d); 4.23-4.19 (1H, m); 4.16-4.07 (2H, m); 4.02 (1H, m); 3.88-3.86 (1H, m); 3.36 (2H, m); 3.06-3.04 (2H, d); 2.31 (3H, s); 2.26 (2H, s); 1.98 (3H, s); 1.74-1.59 (12H, m); 1.34-1.29 (3H, t).

MS: APCI(+ve) 447 (M+1)

### EXAMPLE 187

### 2-(1-Adamantyl)-N-(2-{(3S)-3-[(2-hydroxyethyl)amino]pyrrolidin-1-yl}-6-methylquinolin-5-yl)acetamide dihydrochloride

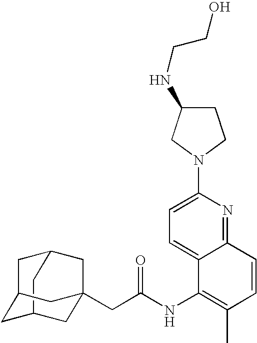

To (3R)-1-{5-[(1-adamantylacetyl)amino]-6-methylquinolin-2-yl}pyrrolidin-3-yl methanesulfonate (Example 185 step (ii)) (280 mg) in N-methylpyrrolidinone (5 mL) was added ethanolamine (2 mL). The mixture was heated to 80° C. in a sealed tube and stirred 20 hours. The mixture was cooled to room temperature and poured into 2N sodium hydroxide solution (50 mL). The resulting solution was extracted with ethyl acetate (3×50 mL) and the combined organic extracts washed with brine (3×50 mL), dried over anhydrous sodium sulfate, filtered and concentrated. The residue was purified by reverse-phase hplc using 0.1% aqueous trifluoroacetic acid:acetonitrile (95:5 to 50:50 over 10 mins, Xterra column). The resulting oil was dissolved in dichloromethane (5 mL) and a 1M solution of hydrogen chloride in diethyl ether (5 mL) was added. The solvents were removed in vacuo and the solid was dried under vacuum to afford the title compound (0.080 g).

¹H NMR (400 MHz, DMSO-d₆, 90° C.) δ 9.62 (1H, s); 8.20-8.18 (1H, d); 8.10-8.08 (1H, d); 7.65-7.63 (1H, d); 7.22-7.19 (1H, d); 4.19-4.09 (4H, m); 3.86-3.83 (1H, m); 3.79-3.76 (2H, m); 3.33 (2H, m); 3.13 (2H, s); 2.31 (3H, s); 2.26 (2H, s); 1.98 (3H, s); 1.74-1.64 (12H, m).

MS: APCI(+ve) 463 (M+1)

### EXAMPLE 188

### 2-(1-Adamantyl)-N-(2-{(3S)-3-[(3-hydroxypropyl)amino]pyrrolidin-1-yl}-6-methylquinolin-5-yl)acetamide dihydrochloride

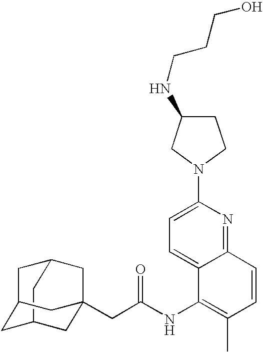

To (3R)-1-{5-[(1-adamantylacetyl)amino]-6-methylquinolin-2-yl}pyrrolidin-3-yl methanesulfonate (Example 185 step (ii)) (280 mg) in N-methylpyrrolidinone (5 mL) was added propanolamine (2 mL). The mixture was heated to 80° C. in a sealed tube and stirred 20 hours. The mixture was cooled to room temperature and poured into 2N sodium hydroxide solution (50 mL). The resulting solution was extracted with ethyl acetate (3×50 mL) and the combined organic extracts washed with brine (3×50 mL), dried over anhydrous sodium sulfate, filtered and concentrated. The residue was purified by reverse-phase hplc using 0.1% aqueous trifluoroacetic acid:acetonitrile (95:5 to 50:50 over 10 mins, Xterra column). The resulting oil was dissolved in dichloromethane (5 mL) and a 1M solution of hydrogen chloride in diethyl ether (5 mL) was added. The solvents were removed in vacuo and the solid was dried under vacuum to afford the title compound (0.150 g).

¹H NMR (400 z, DMSO-d₆, 90° C.) δ 9.73-9.59 (2H, m); 8.21-8.19 (1H, d); 8.13-8.10 (1H, d); 7.67-7.65 (1H, d); 7.25-7.22 (1H, d); 4.21-4.06 (4H, m); 3.88 (1H, m); 3.56 (2H, m); 3.27 (2H, m); 3.08 (2H, m); 2.31 (3H, s); 2.26 (2H, s); 1.98 (3H, s); 1.92-1.90 (2H, m); 1.75-1.64 (12H, m).

MS: APCI(+ve) 477 (M+1)

### EXAMPLE 189

### 2-(1-Adamantyl)-N-{6-chloro-2-[(3R)-3,4-dihydroxybutyl]quinolin-5-yl}acetamide hydrochloride

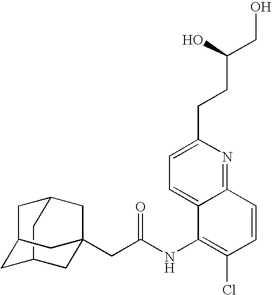

### (i) (5R)-2,2,3,3,8,8,9,9-octamethyl-5-vinyl-4,7-dioxa-3,8-disiladecane

tert-Butyl(chloro)dimethylsilane (6.84 g) was added to a solution of (2R)-but-3-ene-1,2-diol (4 g) in dimethylformamide (100 mL) cooled at 0° C., followed by the addition of imidazole (6.12 g). After one hour at 0° C., the reaction was allowed to warm up to room temperature and stirred for a further 2 hours. The reaction was partitioned between ether and water. The aqueous phase was further extracted with ether and the combined organics were washed with water (3×250 mL) then brine (250 mL). The ether phase was dried over magnesium sulphate, filtered and evaporated to dryness to give 11.15 g of the title compound as a clear oil.

¹H NMR (300 MHz, CDCl₃) δ 5.85 (ddd, 1H), 5.25 (dt, 1H), 5.09 (dt, 1H), 4.14 (q, 1H), 3.48 (ddd, 2H), 0.88 (d, 18H), 0.05 (d, 6H), 0.03 (s, 6H)

### (ii) 2-(1-Adamantyl)-N-{6-chloro-2-[(3R)-3,4-dihydroxybutyl]quinolin-5-yl}acetamide hydrochloride

By the method outlined in Example 161, a solution of (5R)-2,2,3,3,8,8,9,9-octamethyl-5-vinyl-4,7-dioxa-3,8-disiladecan (Example 189 step (i)) (1.62 g) in 9-borabicyclo[3.3.1]nonane (20.6 mL of a 0.5M solution in tetrahydrofuran) was heated at reflux under nitrogen for 10 hours. The solution was cooled to room temperature and a solution of tripotassium orthophosphate monohydrate (2.94 g in 10 mL of water) was added. The mixture was stirred for 5 minutes and a warm solution of 2-(1-adamantyl)-N-(2,6-dichloroquinolin-5-yl)acetamide (Example 175 step (ii)) (1 g) in anhydrous 1-methyl-2-pyrrolidinone (10 mL) was added followed by tetrakistriphenylphosphine palladium (0) (200 mg). The mixture was heated to 80° C. for 3 hours, cooled down to room temperature, filtered through celite. The filtrate was concentrated to half its original volume, diluted with water (25 mL) and extracted into ethyl acetate (3×25 mL). The combined extracts were washed further with water (3×25 mL), brine (25 mL), dried over anhydrous magnesium sulphate, filtered and concentrated. The dark residue was purified by flash column chromatography on silica gel eluting with neat dichloromethane. 124 mg of the purified intermediate was dissolved in dichloromethane and treated with a solution of hydrogen chloride at 4 M in dioxane (1 mL) and the reaction was stirred under nitrogen for 14 hours. The precipitate was collected by filtration and dried in a vacuum oven at 45° C. for 14 hours to give 80 mg of the title compound.

¹H NMR (300 MHz, DMSO-d₆, 90° C.) δ 9.79 (s, 1H), 8.41 (d, 1H), 8.11 (d, 1H), 7.93 (d, 1H), 7.72 (d, 1H), 3.54 (ddd, 1H), 3.36 (ddd, 2H), 3.22 (ddd, 1H), 3.13 (ddd, 1H), 2.27 (s, 2H), 2.09-1.94 (m, 4H), 1.89-1.78 (m, 1H), 1.76 (d, 7H), 1.69 (dd, 6H)

MS: APCI(+ve) 443 (M+1)

MP: 147-150° C.

### EXAMPLE 190

### 2-(1-Adamantyl)-N-{6-chloro-2-[(3R)-3-hydroxy-4-(methylamino)butyl]quinolin-5-yl}acetamide dihydrochloride

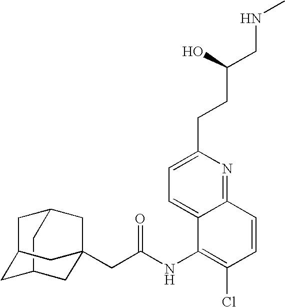

2-(1-Adamantyl)-N-{6-chloro-2-[(3R)-3,4-dihydroxybutyl]quinolin-5-yl}acetamide (Example 189) (229 mg) and triethylamine (0.1 mL) in dichloromethane (2 mL) and tetrahydrofuran (6 mL) were stirred under nitrogen and cooled to 5° C. Methanesulfonyl chloride (0.038 mL) was added to this and the mixture was stirred at 5° C. for 30 mins, poured into saturated sodium bicarbonate solution (50 mL), extracted with ethyl acetate (3×50 mL), washed with brine (3×50 mL), dried over anhydrous magnesium sulfate, filtered and concentrated to give the mesylate (0.253 g). To this was added methylamine (40% solution in water, 5 mL) and tetrahydrofuran (10 mL). The mixture was heated to 70° C. and stirred 20 hours under nitrogen. The mixture was cooled and the solvents removed in vacuo. The residue was purified by chromatography on silica gel eluting with dichloromethane:methanol:aqueous ammonia (9:1:0.1). The resulting oil was dissolved in dichloromethane (5 mL) and a 1M solution of hydrogen chloride in diethyl ether (4 mL) was added. The solvents were removed in vacuo and the solid was recrystallised from dichloromethane:methanol:diethyl ether:isohexane (1:0.1:2:1) to afford the title compound (0.037 g).

¹H NMR (400 MHz, DMSO-d₆) δ 10.02 (1H, s); 8.60-8.51 (2H, br d); 8.30-8.28 (1H, d); 8.01-7.99 (1H, d); 7.92-7.90 (1H, d); 7.69-7.67 (1H, d); 3.84-3.82 (1H, br m); 3.18-3.02 (3H, m); 2.89-2.83 (1H, m); 2.57-2.51 (2H, m); 2.26 (3H, s); 1.98 (3H, br s); 1.93-1.83 (2H, m); 1.74 (6H, br s); 1.69-1.58 (6H, br AB).

MS: APCI(+ve) 456 (M+1)

### EXAMPLE 191

### 2-(1-Adamantyl)-N-{2-[(3R)-3-aminopyrrolidin-1-yl]-6-methylquinolin-5-yl}acetamide dihydrochloride

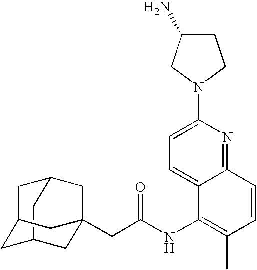

### (i) (3R)-3-Aminopyrrolidine-1-carbaldehyde

A solution of (3R)-pyrrolidin-3-amine (1 g) in methanol (2 mL) was cooled to −65° C. under nitrogen and methyl formate (0.8 mL) was added. The mixture was allowed to warm to −40° C. over 0.5 hour and stirred at −40° C. for 5 hours under nitrogen. The mixture was then allowed to warm to room temperature and concentrated to afford the sub-title compound (1.3 g).

¹H NMR (300 MHz, CDCl₃) δ 8.23 (1H, s); 3.80-2.99 (5H, m); 2.17-2.05 (1H, m); 1.76-1.68 (1H, m); 1.58 (2H, br s).

### (ii) 2-(1-Adamantyl)-N-{2-[(3R)-3-aminopyrrolidin-1-yl]-6-methylquinolin-5-yl}acetamide dihydrochloride

To 2-(1-adamantyl)-N-(2-chloro-6-methylquinolin-5-yl)acetamide (Example 4) (200 mg) and potassium carbonate (400 mg) in N-methylpyrrolidinone (2 mL) was added (3R)-3-aminopyrrolidine-1-carbaldehyde (Example 191 step (i)) (1 g). The mixture was heated to 140° C. in a sealed tube and stirred 20 hours. The mixture was cooled to room temperature and poured into brine (50 mL), extracted with ethyl acetate (3×50 mL) and the combined organic extracts washed with brine (3×50 mL), dried over anhydrous magnesium sulfate, filtered and concentrated. The residue was purified by chromatography on silica gel eluting with dichloromethane:methanol:aqueous ammonia (19:1:0.1) and then by reverse-phase hplc using 0.1% aqueous trifluoroacetic acid:acetonitrile (95:5 to 50:50 over 20 mins, Xterra column). The resulting oil was dissolved in dichloromethane (5 mL) and a 1M solution of hydrogen chloride in diethyl ether (5 mL) was added. The solvents were removed in vacuo and the solid was dried under vacuum to afford the title compound (0.075 g).

¹H NMR (400 MHz, CD₃OD, 50° C.) δ 8.36 (1H, br s); 7.95 (1H, br s); 7.78 (1H, br s); 7.31 (1H, br s); 4.28-4.06 (4H, m); 3.32 (2H, br s); 2.70 (1H, br s); 2.43 (3H, s); 2.35 (2H, s); 2.04 (3H, s); 1.84-1.73 (12H, m).

MS: APCI(+ve) 419 (M+1)

### EXAMPLE 192

### 2-(1-Adamantyl)-N-(6-chloro-2-{[2-(methylamino)ethyl]amino}quinolin-5-yl)acetamide dihydrochloride

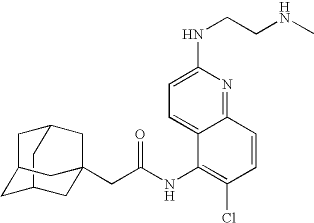

A solution of 2-(1-adamantyl)-N-(2,6-dichloroquinolin-5-yl)acetamide (Example 175 step (ii)) (200 mg) in 1-methyl-2-pyrrolidinone (2 mL) was treated with tert-butyl 2-aminoethyl(methyl)carbamate (495 mg) and potassium carbonate (80 mg) following the procedure outlined in Example 6. The resulting solid was dissolved in the minimum amount of methanol and ethyl acetate was added until a white precipitate had formed. The solid was collected by filtration and dried in a vacuum oven at 10° C. for 3 hours to give 55 mg of the title compound as a solid.

¹H NMR (400 MHz, DMSO-d₆, 90° C.) δ 9.64 (s, 1H), 9.10 (s, 2H), 8.05-7.94 (m, 2H), 7.74 (d, 1H), 7.14 (d, 1H), 3.96 (t, 2H), 3.25 (t, 2H), 2.63 (s, 3H), 2.23 (s, 2H), 1.97 (s, 3H), 1.74 (d, 6H), 1.68 (dd, 6H)

MS: APCI(+ve) 427 (M+1).

MP: 232-234° C.

### EXAMPLE 193

### 2-(1-Adamantyl)-N-(6-chloro-2-{methyl[3-(methylamino)propyl]amino}quinolin-5-yl)acetamide dihydrochloride

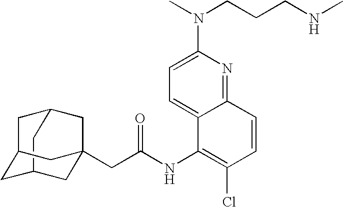

A solution of 2-(1-adamantyl)-N-(2,6-dichloroquinolin-5-yl)acetamide (200 mg) in 1-methyl-2-pyrrolidinone (2 mL) was treated with N,N′-dimethylpropane-1,3-diamine (1.23 g) and potassium carbonate (70 mg) following the procedure outlined in Example 6. The obtained solid was purified on silica gel eluting with a mixture of 7N methanolic ammonia, methanol and dichloromethane in the respective ratio 0.2:0.8:99 increased to 0.6:2.4:97. The residue obtained after evaporation of the fraction of interest was dissolved in dichloromethane and treated with a solution of hydrogen chloride at 4 M in 1,4-dioxane. The solvent was evaporated under vacuum, the residue was dissolved in the minimum amount of hot methanol and ethyl acetate was added until a white precipitate had formed. The solid was collected by filtration and purified further by reverse phase HPLC using acetonitrile from 5% to 40% in 0.1% aqueous trifluoroacetic acid. The fractions of interest were combined, evaporated, dissolved in methanol, treated with a solution of hydrogen chloride at 4 M in 1,4-dioxane, concentrated in vacuo and dried in a vacuum oven at 50° C. for 3 hours to give 70 mg of the title compound as a white solid.

¹H NMR (400 MHz, DMSO-d₆, 90° C.) δ 9.64 (s, 1H), 9.07 (s, 2H), 8.03 (d, 1H), 7.98 (s, 1H), 7.69 (d, 1H), 7.36 (d, 1H), 3.90 (t, 2H), 3.29 (s, 3H), 2.98 (s, 2H), 2.54 (s, 3H), 2.24 (s, 2H), 2.05 (quintet, 2H), 1.97 (s, 3H), 1.75 (s, 6H), 1.68 (dd, 6H)

MS: APCI(+ve) 455 (M+1).

### EXAMPLE 194

### 2-(1-Adamantyl)-N-(6-chloro-2-{3-[(3-hydroxypropyl)amino]propyl}quinolin-5-yl)acetamide dihydrochloride

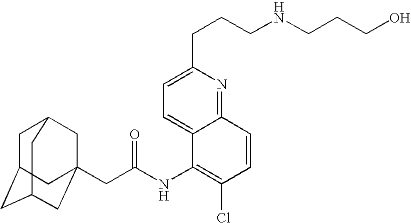

By the method outlined in Example 161, a solution of tert-butyl allyl(3-{[tert-butyl(dimethyl)silyl]oxy}propyl)carbamate (446 mg) in 9-boroabicyclo[3.3.1]nonane (5 mL of a 0.5M solution in tetrahydrofuran) was heated at reflux under nitrogen for 10 hours. The solution was cooled to room temperature and a solution of tripotassium orthophosphate monohydrate (700 mg in 1 mL of water) was added. The mixture was stirred for 5 minutes and a warm solution of 2-(1-adamantyl)-N-(2,6-dichloroquinolin-5-yl)acetamide (Example 175 step (ii)) (350 mg) and tetrakistriphenylphosphine palladium(0) (50 mg) in anhydrous N,N-dimethylformamide (3 mL) was added. The mixture was heated to 80° C. stirred for 3 hours, diluted with water (25 mL) and extracted into ethyl acetate (3×25 mL). The combined extracts were washed further with brine (25 mL) dried over anhydrous magnesium sulphate, filtered and concentrated. The residue was purified by chromatography on silica gel eluting with neat dichloromethane, 7 N methanolic ammonia in dichloromethane from 0.5% to 10%, then 5% methanol in dichloromethane. The isolated material was dissolved in dichloromethane and treated with a solution of hydrogen chloride in dioxane (10 ml of a 4 M solution) and stirred for four hours under nitrogen. The precipitate was collected by vacuum filtration, dissolved in minimum amount of hot methanol and ethyl acetate was added slowly until a white precipitate started to form. The solid was allowed to crystallise slowly then collected by vacuum filtration and dried in a vacuum oven at 45° C. for four hours to afford the title compound as a white solid (267 mg).

¹H NMR (500 MHz, DMSO-d₆) δ 10.29 (s, 1H), 9.07 (s, 2H), 8.58 (d, 1H), 8.23 (d, 1H), 8.06 (d, 1H), 7.89 (d, 1H), 3.48 (t, 2H), 3.26 (t, 2H), 3.04-2.90 (m, 4H), 2.30 (s, 2H), 2.23 (quintet, 2H), 1.98 (s, 3H), 1.80 (dt, 2H), 1.75 (d, 6H), 1.67 (dd, 6H).

MS: APCI(+ve) 470 (M+1)

### EXAMPLE 195

### 2-(1-Adamantyl)-N-[6-chloro-2-({3-[(2-hydroxyethyl)amino]propyl}amino)quinolin-5-yl]acetamide dihydrochloride

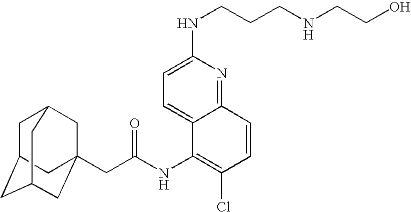

By the method outlined in example 174 using 2-(1-adamantyl)-N-(2,6-dichloroquinolin-5-yl)acetamide (Example 175 step (ii)) (200 mg), potassium carbonate (150 mg) and 2-[(3-aminopropyl)amino]ethanol (365 mg) to afford the title compound (45 mg) as a white solid.

¹H NMR (400 MHz, DMSO-d₆, 90° C.) δ 9.67 (1H, s); 8.06 (1H, s); 8.00 (1H, d); 7.76 (1H, d); 7.20 (1H, d); 3.78 (2H, t); 3.71 (2H, t); 3.11 (2H, t); 3.02 (2H, t); 2.23 (2H, s); 2.08 (2H, quintet); 1.97 (3H, s); 1.74 (6H, d); 1.67 (6H, dd)

MS: APCI(+ve) 471 (M+1)

MP: 264-266° C.

### EXAMPLE 196

### 2-(1-Adamantyl)-N-(6-chloro-2-{[4-(2-hydroxyethyl)piperazin-1-yl]methyl}quinolin-5-yl)acetamide

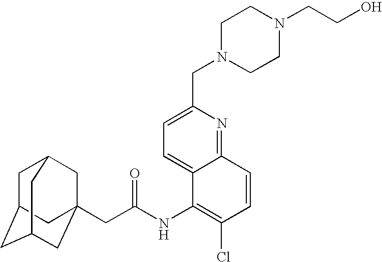

### (i) 2-(1-Adamantyl)-N-(6-chloro-2-vinylquinolin-5-yl)acetamide

A suspension of 2-(1-adamantyl)-N-(2,6-dichloroquinolin-5-yl)acetamide (Example 175 step (ii)) (1 g) in dimethylformamide (15 mL) was treated with tributyl(vinyl)tin (2.25 mL), 2,6-di-tert-butyl-4-methylphenol (50 mg) and dichlorobis(triphenylphosphine) palladium (54 mg). The reaction was stirred at 80° C. under nitrogen for 16 hours, cooled to room temperature, diluted with ethyl acetate and filtered through celite that was then washed thoroughly with water. The aqueous was extracted further with ethyl acetate and the combined organic layers were washed with brine (2×), dried over magnesium sulphate, filtered and evaporated. The residue was purified by flash column chromatography on silica eluting with a mixture of methanol at 0.1% in dichloromethane increased to 0.25% to give 850 mg of the title compound.

MS: APCI(+ve) 381 (M+1).

### (ii) 2-(1-Adamantyl)-N-(6-chloro-2-formylquinolin-5-yl)acetamide

Ozone was bubbled through a solution of 2-(1-adamantyl)-N-(6-chloro-2-vinylquinolin-5-yl)acetamide (Example 196 step (i)) in dichloromethane (50 mL) and acetic acid (1 mL) at −78° C. for 2 hours. Dimethylsulfide (0.5 mL) was added and the solution was allowed to warm up overnight to room temperature. Saturated aqueous sodium bicarbonate was added to the reaction that was then stirred vigorously. The aqueous phase was separated and further extracted with dichloromethane. The combined organics were washed with brine, dried over magnesium sulphate, filtered and evaporated. The residue was purified by flash column chromatography on silica eluting with dichloromethane then 1% methanol in dichloromethane to give 700 mg of the title product.

MS: APCI(+ve) 383 (M+1).

### (iii) 2-(1-Adamantyl)-N-(6-chloro-2-{[4-(2-hydroxyethyl)piperazin-1-yl]methyl}quinolin-5-yl)acetamide

1-(2-Hydroxyethyl)piperazine (100 μL) was added to a solution of 2-(1-adamantyl)-N-(6-chloro-2-formylquinolin-5-yl)acetamide (Example 196 step (ii)) (150 mg) in methanol (5 mL) and acetic acid (200 μL). After stirring the solution for 5 minutes, sodium triacetoxyborohydride (165 mg) was added and the reaction was stirred overnight. Further sodium triacetoxyborohydride (330 mg) was added and the reaction was stirred for 80 hours. The reaction was concentrated in vacuo and purified on silica gel eluting with a mixture of methanol in dichloromethane from 3% to 5%, followed by 7 N methanolic ammonia at 5% in dichloromethane to afford 25 mg of the title compound as a pale yellow solid.

¹H NMR (400 MHz, CD₃OD) δ 8.29 (dd, 1H), 7.98 (dd, 1H), 7.83 (d, 1H), 7.76 (d, 1H), 3.84 (s, 2H), 3.67 (t, 2H), 2.61 (s, 8H), 2.55 (t, 2H), 2.33 (s, 2H), 2.03 (s, 3H), 1.85 (d, 6H), 1.77 (dd, 6H)

MS: APCI(+ve) 497 (M+1)

### EXAMPLE 197

### 2-(1-Adamantyl)-N-[6-chloro-2-{[2-(methylamino)ethyl]amino}methyl)quinolin-5-yl]acetamide

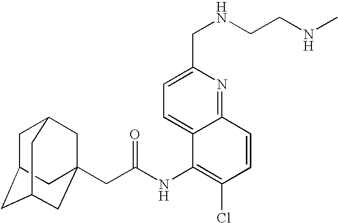

tert-Butyl 2-aminoethyl(methyl)carbamate (136 mg) was added to a solution of 2-(1-adamantyl)-N-(6-chloro-2-formylquinolin-5-yl)acetamide (Example 196 step (ii)) (150 mg) in methanol (5 mL) and acetic acid (100 μL). After stirring the solution for 5 minutes, sodium triacetoxyborohydride (165 mg) was added and the reaction was stirred overnight. The reaction was concentrated in vacuo and the residue was dissolved in dichloromethane, washed with water then brine, dried over magnesium sulphate, filtered and evaporated to dryness. The residue was purified on silica gel eluting with a mixture of 7 N methanolic ammonia, methanol and dichloromethane in the respective ratio 0.2:0.8:99 increased to 0.6:2.4:97. The solid obtained was dissolved in methanol (20 mL) and treated with aqueous hydrochloric acid (20 mL, 2 M), stirred overnight then neutralised with saturated aqueous sodium bicarbonate. The reaction mixture was concentrated and then extracted with ether (3×30 mL). The combined organics were washed with water, brine, dried over sodium carbonate, filtered and evaporated. The residue was purified over silica by flash column chromatography eluting with methanol in dichloromethane from 0% to 15% followed by 7 N methanolic ammonia in dichloromethane from 1% gradually increased to 10% to give the title compound (25 mg).

¹H NMR (400 MHz, CD₃OD) δ 8.29 (d, 1H), 7.99 (d, 1H), 7.83 (d, 1H), 7.66 (d, 1H), 4.09 (s, 2H), 2.84-2.79 (m, 2H), 2.76-2.72 (m, 2H), 2.39 (s, 3H), 2.33 (s, 2H), 2.03 (s, 3H), 1.85 (d, 6H), 1.77 (dd, 6H).

MS: APCI(+ve) 441 (M+1)

### EXAMPLE 198

### 2-(1-Adamantyl)-N-{6-chloro-2-[({2-[(2-hydroxyethyl)amino]ethyl}amino)methyl]quinolin-5-yl}acetamide

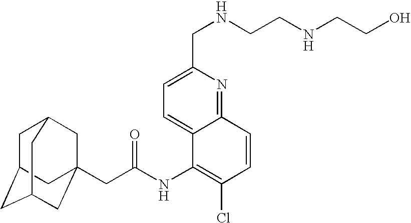

tert-Butyl 2-aminoethyl(2-hydroxyethyl)carbamate (267 mg) was added to a solution of 2-(1-adamantyl)-N-(6-chloro-2-formylquinolin-5-yl)acetamide (Example 196 step (ii)) (250 mg) in methanol (8 mL) and acetic acid (150 μL). After stirring the solution overnight, sodium triacetoxyborohydride (277 mg) was added and the reaction was stirred for 1.5 hours. The solution was flushed through silica that was then washed thoroughly with methanol. The methanolic solution was treated with aqueous hydrochloric acid (20 mL, 2 M) and stirred for 24 hours. The solution was neutralised with saturated aqueous sodium bicarbonate and extracted with dichloromethane (2×20 mL). The combined organics were dried over magnesium sulphate, filtered, concentrated in vacuo and the residue was purified by reverse phase HPLC using acetonitrile and 0.1% aqueous trifluoroacetic acid with a gradient from 5% to 40% in organic phase. The purified product was neutralised and extracted with dichloromethane. The organic phase was dried over sodium carbonate, filtered, evaporated and dried in a vacuum oven to afford 126 mg of the title compound.

¹H NMR (400 MHz, CD₃OD) δ 8.19 (d, 1H), 7.90 (d, 1H), 7.73 (d, 1H), 7.58 (d, 1H), 4.00 (s, 2H), 3.56 (t, 2H), 2.72 (dd, 4H), 2.63 (t, 2H), 2.24 (s, 2H), 1.93 (s, 3H), 1.76 (d, 6H), 1.68 (dd, 6H).

MS: APCI(+ve) 471 (M+1)

### EXAMPLE 199

### 2-(1-Adamantyl)-N-[6-chloro-2-({[3-(methylamino)propyl]amino}methyl)quinolin-5-yl]acetamide bis(trifluoroacetate)

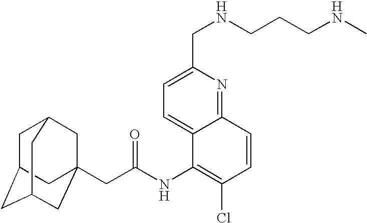

tert-Butyl 3-aminopropyl(methyl)carbamate (246 mg) was added to a solution of 2-(1-adamantyl)-N-(6-chloro-2-formylquinolin-5-yl)acetamide (Example 196 step (i)) (250 mg) in methanol (8 mL) and acetic acid (150 μL). After stirring the solution overnight, sodium triacetoxyborohydride (277 mg) was added and the reaction was stirred for 1.5 hours. The solution was flushed through silica that was then washed thoroughly with methanol. The methanolic solution was treated with aqueous hydrochloric acid (20 mL, 2 M) and stirred for 24 hours. The solution was neutralised with saturated aqueous sodium bicarbonate and extracted with dichloromethane (2×20 mL). The combined organics were dried over magnesium sulphate, filtered, concentrated in vacuo and the residue was purified by reverse phase HPLC using acetonitrile and 0.1% aqueous trifluoroacetic acid with a gradient from 5% to 40% in organic phase. The purified product was concentrated and dried in a vacuum oven at 60° C. to afford the title compound (105 mg).

¹H NMR (400 MHz, DMSO-d₆, 90° C.) δ 9.70 (s, 1H), 8.31 (d, 1H), 7.99 (d, 1H), 7.89 (d, 1H), 7.66 (d, 1H), 4.52 (s, 2H), 3.17 (t, 2H), 3.04 (t, 2H), 2.59 (s, 3H), 2.27 (s, 2H), 2.06 (quintet, 2H), 1.98 (s, 3H), 1.77 (s, 6H), 1.69 (dd, 6H)

MS: APCI(+ve) 455 (M+1)

### EXAMPLE 200

### 2-(1-Adamantyl)-N-(6-chloro-2-{[(3R)-pyrrolidin-3-ylamino]methyl}quinolin-5-yl)acetamide tris(trifluoroacetate)

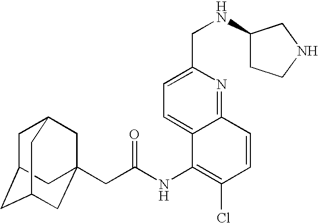

tert-Butyl (3R)-3-aminopyrrolidine-1-carboxylate (244 mg) was added to a solution of 2-(1-adamantyl)-N-(6-chloro-2-formylquinolin-5-yl)acetamide (Example 196 step (ii)) (250 mg) in methanol (8 mL) and acetic acid (150 μL). After stirring the solution overnight, sodium triacetoxyborohydride (277 mg) was added and the reaction was stirred for 1.5 hours. Further sodium triacetoxyborohydride (831 mg) was added and the reaction was stirred for 3 hours. The solution was flushed through silica which was then washed thoroughly with methanol. The methanolic solution was treated with aqueous hydrochloric acid (20 mL, 2 M) and stirred for 48 hours. The solution was neutralised with saturated aqueous sodium bicarbonate and extracted with dichloromethane (2×20 mL). The combined organics were dried over magnesium sulphate, filtered, concentrated in vacuo and the residue was purified by reverse phase HPLC using acetonitrile and 0.1% aqueous trifluoroacetic acid with a gradient from 5% to 40% in organic phase. The purified product was concentrated and dried in a vacuum oven at 60° C. to afford the title compound (65 mg).

¹H NMR (300 MHz, DMSO-d₆, 90° C.) δ 9.66 (s, 1H), 8.28 (dd, 1H), 7.97 (d, 1H), 7.86 (d, 1H), 7.66 (d, 1H), 4.42 (s, 2H), 3.93 (quintet, 1H), 3.54-3.21 (m, 4H), 2.27 (s, 2H), 2.15 (quintet, 1H), 1.98 (s, 3H), 1.77 (d, 6H), 1.69 (dd, 6H)

MS: APCI(+ve) 453 (M+1)

MP: 90-91° C.

### EXAMPLE 201

### 2-(1-Adamantyl)-N-[2-({3-[(pyridin-2-ylmethyl)amino]propyl}amino)quinolin-5-yl]acetamide

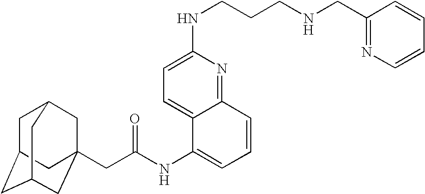

Pyridine-2-carbaldehyde (39 mg) was added to a solution of 2-(1-adamantyl)-N-{2-[(3-aminopropyl)amino]quinolin-5-yl}acetamide (Example 16) (50 mg) in methanol (5 mL) and acetic acid (10 μL). After stirring for 2 hours, sodium triacetoxyborohydride (127 mg) was added and the reaction was stirred over night. Further sodium triacetoxyborohydride (170 mg) was added and the reaction was stirred for 24 hours. The reaction mixture was diluted with saturated aqueous sodium bicarbonate and extracted with ethyl acetate (3×20 mL). The combined organic extracts were concentrated in vacuo and purified by SCX resin. Further purification was performed by flash column chromatography eluting with 0.1 N methanolic ammonia in dichloromethane from 1% gradually increased to 100% to give the title compound (25 mg).

¹H NMR (400 MHz, CDCl₃) δ 8.56 (d, 1H), 7.80 (d, 1H), 7.63 (td, 1H), 7.51-7.44 (m, 3H), 7.29 (d, 1H), 7.16 (dd, 1H), 6.60 (d, 1H), 5.50 (m, 1H), 3.91 (s, 2H), 3.61 (q, 2H), 2.81 (t, 2H) 2.20 (s, 2H), 2.02 (m, 3H), 1.87 (q, 2H), 1.78-1.63 (m, 12H).

MS: APCI(+ve) 484 (M+1)

### EXAMPLE 202

### 2-(1-Adamantyl)-N-[2-({2-[(2-hydroxyethyl)amino]propyl}amino)-6-methylquinolin-5-yl]acetamide hydrochloride

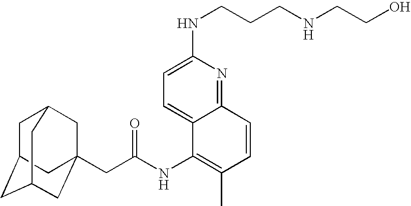

Prepared by the method of Example 174 step (i)/(ii) using 2-(1-adamantyl)-N-(2-chloro-6-methylquinolin-5-yl)acetamide (Example 4) (238 mg) and 2-[(3-aminopropyl)amino]ethanol (2 mL) to give the title compound (7 mg).

¹H NMR (300 MHz, DMSO-d₆) δ 9.54 (s, 1H), 8.08 (d, 1H), 8.00 (s, 1H), 7.61 (d, 1H), 7.18 (d, 1H), 3.79 (t, 2H), 3.71 (dd, 2H), 3.12 (t, 2H), 3.03 (t, 2H), 2.30 (s, 3H), 2.24 (s, 2H), 2.09 (q, 2H), 1.97 (m, 3H), 1.75-1.60 (m, 12H).

MS: APCI(+ve) 451.2 (M+1)

MP: 217-222° C.

### EXAMPLE 203

### N-(1-Adamantylmethyl)-2-[3-(methylamino)propyl]quinoline-5-carboxamide dihydrochloride

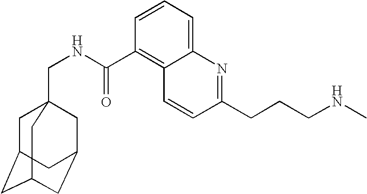

By the method outlined in Example 172, a solution of tert-butyl allyl(methyl)carbamate (0.2 g) in 9-borabicyclo[3.3.1]nonane (4 ml of a 0.5M solution in tetrahydrofuran) was heated at reflux under nitrogen for 2 hours. The solution was cooled to room temperature and potassium phosphate (1 ml of a 2.5M solution in water) was added. The mixture was stirred for 15 minutes and a solution of N-(1-adamantylmethyl)-2-chloroquinoline-5-carboxamide (0.300 g) and tetrakistriphenylphosphine palladium(0) (0.015 g) in anhydrous N,N-dimethylformamide (1.5 ml) was added. The mixture was heated to 60° C. stirred for 2 hours, diluted with saturated brine (25 ml) and extracted into ethyl acetate (3×25 ml). The combined extracts were dried over anhydrous magnesium sulphate, filtered and concentrated. The residue was purified by chromatography on silica gel eluting 5% methanol in dichloromethane. The isolated material was dissolved in a solution of hydrogen chloride in dioxane (10 ml of a 4M solution) and concentrated; the resultant solid was re-crystallised from methanol-ethyl acetate to afford the title compound as a white solid (60 mg).

¹H NMR (400 MHz, DMSO-d₆) δ 8.90 (2H, m); 8.73 (1H, d); 8.30 (1H, m); 8.17 (1H, d); 7.84 (1H, t); 7.78 (1H, d); 7.64 (1H, d); 3.15 (2H, d); 3.08 (2H, d); 3.00 (2H, m); 2.56 (3H, m); 2.19 (2H, m), 1.97 (3H, m); 1.75-1.61 (6H, m); 1.59 (6H, m).

MS: APCI(+ve) 392.3 (M+1)

MP: 158-160° C.

### EXAMPLE 204

### 2-(1-Adamantyl)-N-(6-methyl-2-{[3-(methylamino)propyl]amino}quinolin-5-yl)acetamide

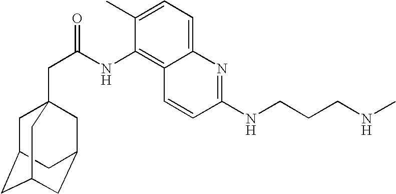

### (i) tert-Butyl[3-({5-[(1-adamantylacetyl)amino]-6-methylquinolin-2-yl}amino)propyl]methylcarbamate

To a solution of 2-(1-adamantyl)-N-(2-chloro-6-methylquinolin-5-yl)acetamide (Example 4) (200 mg) in 1-methyl-2-pyrrolidinone was added tert-butyl 2-aminopropyl(methyl)carbamate (500 mg) and potassium carbonate (290 mg). The mixture was heated to 120° C. for 18 hours. The cooled reaction mixture was partitioned between water and dichloromethane, and the organic layer separated. The aqueous layer was further extracted with dichloromethane and the combined organic layers, dried over anhydrous magnesium sulfate, filtered and concentrated. The residue was purified by chromatography on silica gel eluting with methanol/dichloromethane (1/20) and then by ethyl acetate/isohexane (3/10) to afford the title compound as a solid (120 mg).

¹H NMR (400 MHz, DMSO-d₆) δ 9.43 (1H, s); 7.74 (1H, d); 7.33 (2H, s); 6.88 (1H, t); 6.72 (1H, d), 3.38-3.22 (4H, m); 2.80 (3H, s); 2.22 (3H, s); 2.18 (2H, s); 1.97 (3H, s); 1.80-1.60 (14H, m); 1.38 (9H, s).

MS: APCI(+ve) 521 (M+1).

MP: 232-234° C.

### (ii) 2-(1-Adamantyl)-N-(6-methyl-2-{[3-(methylamino)propyl]amino}quinolin-5-yl)acetamide

To a solution of tert-butyl[3-({5-[(1-adamantylacetyl)amino]-6-methylquinolin-2-yl}amino)propyl]methylcarbamate in methanol (1 mL) and dichloromethane (3 mL) was added hydrochloric acid (4M in dioxane, 2 mL). The resultant mixture was stirred for 3 hours and then evaporated to dryness. The crude product was recrystallised from methanol/ethyl acetate to give the title compound (100 mg).

¹H NMR (400 MHz, DMSO-d₆) δ 9.57 (1H, s); 9.10 (2H, s); 8.08 (1H, d); 8.04 (1H, s); 7.61 (1H, d); 7.20 (1H, d); 3.32 (2H, t); 3.07 (2H, t); 2.56 (3H, s); 2.29 (3H, s); 2.25 (2H, s); 2.06 (2H, quint.); 1.98 (3H, s); 1.82-1.62 (12H, m).

MS: APCI(+ve) 421.3 (M+1).

MP: 217-224° C.

### EXAMPLE 205

### 2-(1-Adamantyl)-N-(2-{2-[(3-hydroxypropyl)amino]ethyl}-6-methylquinolin-5-yl)acetamide dihydrochloride

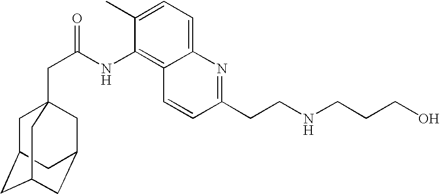

### (i) 2-(1-Adamantyl)-N-(6-methyl-2-vinylquinolin-5-yl)acetamide

Prepared by the method of Example 196 step (i) using 2-(1-adamantyl)-N-(2-chloro-6-methylquinolin-5-yl)acetamide (Example 4) to afford the title compound (100 mg).

MS: APCI(+ve) 361 (M+1).

### (ii) 2-(1-Adamantyl)-N-(2-{2-[(3-hydroxypropyl)amino]ethyl}-6-methylquinolin-5-yl)acetamide dihydrochloride

To a solution of 2-(1-adamantyl)-N-(6-methyl-2-vinylquinolin-5-yl)acetamide (Example 205 step (i) (100 mg) in acetic acid (3 mL) was added 3-aminopropan-1-ol (500 mg). The mixture was heated to 90° C. for 4 hours and cooled to room temperature. The mixture was poured into dichloromethane and aqueous sodium bicarbonate and the layers separated. The aqueous layer was further extracted with dichloromethane and the combined organic layers, dried over anhydrous magnesium sulfate, filtered and concentrated. The residue was purified by chromatography on silica gel eluting with methanol/dichloromethane/aqueous ammonia (10/90/1). The resultant oil was dissolved in methanol and hydrochloric acid added (4M in 1,4-dioxane, 1 mL). The mixture was stirred for 1 hour and then evaporated to dryness. The residue was recrystallised from ethanol/ethyl acetate to afford the title compound as a solid (22 mg).

1H NMR (300 MHz, DMSO-d₆) δ 9.92 (1H, s); 9.06 (2H, m); 8.45 (1H, d); 8.05 (1H, s); 7.82 (1H, d); 7.73 (1H, d); 3.56-3.45 (6H, m); 3.07 (2H, m); 2.39 (3H, s); 2.28 (2H, s); 1.98 (3H, s); 1.82 (2H, quint.); 1.74 (6H, d); 1.72-1.60 (6H, m).

MS: APCI(+ve) 436 (M+1).

MP: 175-178° C.

### EXAMPLE 206

### 2-(1-Adamantyl)-N-[6-chloro-2-(piperazin-1-ylmethyl)quinolin-5-yl]acetamide trifluoroacetate

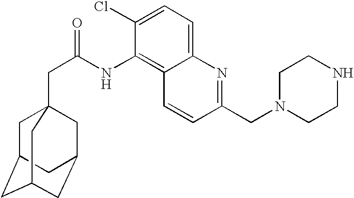

To 2-(1-Adamantyl)-N-(6-chloro-2-formylquinolin-5-yl)acetamide (Example 196 step (ii)) (300 mg) in methanol (5 mL) with acetic acid (100 μL) was added tert-butyl piperazine-1-carboxylate (290 mg). The mixture was stirred at room temperature for 2 hours and triacetoxyborohydride (600 mg) added. The mixture was stirred over night and then poured into saturated aqueous sodium bicarbonate. The organic layer was separated and the aqueous layer extracted with dichloromethane. The combined organic layers were dried over magnesium sulfate, filtered and concentrated. The residue was dissolved in methanol and 2M hydrochloric acid (10 mL) added. The resulting solution was stirred for 24 hours and evaporated to give a crude residue which was purified by reverse phase hplc eluting with 0.1M aqueous trifluoroacetic acid in water/acetonitrile to afford the title compound (130 mg) as a white solid as its trifluoroacetate salt.

¹H NMR (500 MHz, DMSO-d₆) δ 9.96 (1H, s); 8.83 (2H, s); 8.26 (1H, d); 7.99 (1H, d); 7.89 (1H, d); 7.72 (1H, d); 4.10 (2H, s); 3.23 (4H, m); 2.95 (4H, m); 2.26 (2H, s); 1.98 (3H, s); 1.74 (6H, m); 1.74-1.60 (6H, m).

MS: APCI(+ve) 453.1 (M+1).

MP: 132-136° C.

### EXAMPLE 207

### 2-(1-Adamantyl)-N-(6-chloro-2-piperazin-1-ylquinolin-5-yl)acetamide

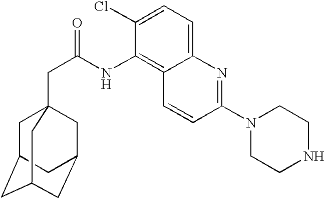

To 2-(1-adamantyl)-N-(2,6-dichloroquinolin-5-yl)acetamide (Example 175 step (ii)) (150 mg) and potassium carbonate (300 mg) in 1-methyl-2-pyrrolidinone (2 mL) was added piperazine (0.88 g). The mixture was heated at 130° C. for 4 hours after which it was cooled and poured into water. The mixture was extracted with dichloromethane and the combined extracts evaporated to give a residue which was then partitioned between water and ethyl acetate. The organic layer was separated and the aqueous layer further extracted with ethylacetate. The combined organic extracts were concentrated to give a residue which was purified by chromatography on silica gel eluting with methanol/dichloromethane/ammonium hydroxide solution (19/80/1) and the resultant product converted to it hydrochloride salt by treatment with hydrochloric acid (4M in dioxane). Recrystallisation from methanol/ethyl acetate afforded the title compound as a solid (100 mg).

¹H NMR (400 MHz, DMSO-d₆) δ 9.97 (1H, s); 9.53 (2H, s); 8.09 (1H, d); 7.91 (1H, s); 7.76 (1H, d); 7.51 (1H, d); 4.10 (4H, s); 3.27 (4H, s); 2.24 (2H, s); 1.97 (3H, s); 1.79-1.58 (12H, m).

MS: APCI(+ve) 439.1 (M+1).

MP: 280-283° C.

### EXAMPLE 208

### N-(1-Adamantylmethyl)-6-chloro-2-{methyl[3-(methylamino)propyl]amino}quinoline-5-carboxamide

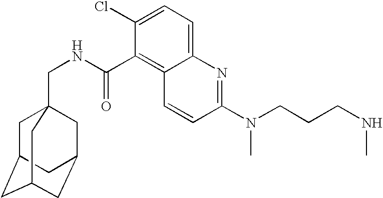

Prepared by the method of Example 6 using N-(1-adamantylmethyl)-2,6-dichloroquinoline-5-carboxamide (100 mg) and N,N′-dimethylpropane-1,3-diamine (500 mg) to afford the product which was purified by chromatography on silica gel eluting with methanol/dichloromethane/ammonium hydroxide solution (9/90/1) and then by reverse phase hplc eluting with 0.05M ammonium acetate in water/acetonitrile to afford the title compound (20 mg) as a white solid.

¹H NMR (400 MHz, DMSO-d₆, TFA) δ 8.87 (2H, s); 8.75 (1H, t); 8.35 (1H, m); 7.98 (1H, d); 7.87 (1H, d); 7.63 (1H, m); 3.95 (2H, m); 3.41 (3H, s); 3.07 (2H, d); 3.02 (2H, m); 2.56 (3H, t); 2.02 (2H, m); 1.97 (3H, m); 1.74-1.55 (12H, m).

MS: APCI(+ve) 455.3 (M+1).

MP: 195-200° C.

### Pharmacological Analysis

Certain compounds such as benzoylbenzoyl adenosine triphosphate (bbATP) are known to be agonists of the P2X₇ receptor, effecting the formation of pores in the plasma membrane (Drug Development Research (1996), 37(3). p. 126). Consequently, when the receptor is activated using bbATP in the presence of ethidium bromide (a fluorescent DNA probe), an increase in the fluorescence of intracellular DNA-bound ethidium bromide is observed. The increase in fluorescence can be used as a measure of P2X₇ receptor activation and therefore to quantify the effect of a compound on the P2X₇ receptor.

In this manner, each of the title compounds of the Examples was tested for antagonist activity at the P2X₇ receptor. Thus, the test was performed in 96-well flat bottomed microtitre plates, the wells being filled with 250 μl of test solution comprising 200 μl of a suspension of THP-1 cells (2.5×10⁶ cells/ml) containing 10⁻⁴ M ethidium bromide, 25 μl of a high potassium buffer solution containing 10⁻⁵ M bbATP, and 25 μl of the high potassium buffer solution containing 3×10⁻⁵ M test compound. The plate was covered with a plastics sheet and incubated at 37° C. for one hour. The plate was then read in a Perkin-Elmer fluorescent plate reader, excitation 520 nm, emission 595 nm, slit widths: Ex 15 nm, Em 20 nm. For the purposes of comparison, bbATP (a P2X₇ receptor agonist) and pyridoxal 5-phosphate (a P2X₇ receptor antagonist) were used separately in the test as controls. From the readings obtained, a pIC₅₀ figure was calculated for each test compound, this figure being the negative logarithm of the concentration of test compound necessary to reduce the bbATP agonist activity by 50%. Each of the compounds of the Examples demonstrated antagonist activity, having a pIC₅₀ figure >4.50. For example, the following table shows the pIC₅₀ figures for a representative selection of compounds:

| Compound of | |
|---|---|
| Example No. | pIC₅₀ |
| 6 | 8.05 |
| 17 | 8.00 |
| 160 | 7.10 |
| 167 | 8.30 |
| 168 | 7.60 |
| 170 | 7.58 |
| 196 | 7.73 |
| 205 | 7.75 |
| 208 | 7.45 |

1. A compound of formula 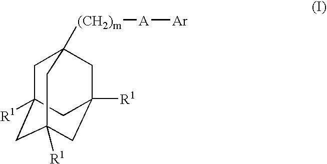
wherein m represents 1, 2 or 3;
each R¹ independently represents a hydrogen or halogen atom;
A represents C(O)NH or NHC(O);
Ar represents a group of formula 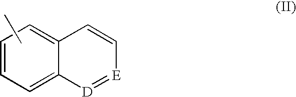
in which one of D and E represents a nitrogen atom and the other of D and E represents CH, the group of formula (II) being optionally substituted by one or more substituent groups R² independently selected from halogen, C₁-C₆ alkyl optionally substituted by at least one substituent selected from hydroxyl, halogen and C₁-C₆ alkoxy,
or a group of formula 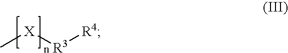
X represents an oxygen or sulphur atom or a group >N—R⁵;
n is 0 or 1;
R³ represents a bond or a C₁-C₅ alkyl group which may be optionally substituted by at least one substituent selected from hydroxyl, halogen, C₁-C₆ alkoxy, C₁-C₆ alkylthio, C₁-C₆ hydroxyalkyl, C₁-C₆ hydroxyalkyloxy, C₁-C₆ alkoxycarbonyl, C₃-C₈ cycloalkyl, phenyl (optionally substituted by at least one substituent selected from halogen, hydroxyl and C₁-C₆ alkylsulphonylamino), benzyl, indolyl (optionally substituted by at least one substituent selected from C₁-C₆ alkoxy), oxopyrrolidinyl, phenoxy, benzodioxolyl, phenoxyphenyl, piperidinyl and benzyloxy;
R⁴ represents hydrogen, hydroxyl or a group —NR⁶R⁷ except that when R³ represents a bond, then R⁴ represents a saturated or unsaturated 4- to 9-membered ring system which may comprise at least one ring heteroatom selected from nitrogen, oxygen and sulphur, the ring system being optionally substituted by at least one substituent selected from hydroxyl, amino, C₁-C₆alkyl, C₁-C₆alkylamino, —NH(CH₂)₂OH, —NH(CH₂)₃OH, C₁-C₆ hydroxyalkyl, benzyl and 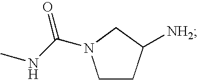
R⁵ represents a hydrogen atom or a C₁-C₅ alkyl group which may be optionally substituted by at least one substituent selected from hydroxyl, halogen and C₁-C₆ alkoxy;
R⁶ and R⁷ each independently represent hydrogen, pyrrolidinyl, C₁-C₆ alkylcarbonyl, C₂-C₇ alkenyl, or C₁-C₇ alkyl optionally substituted with at least one substituent selected from carboxyl, hydroxyl, amino, C₁-C₆ alkylamino, di-C₁-C₆ alkylamino, —NH(CH₂)₂OH, C₁-C₆ alkoxy, C₁-C₆ alkylthio, C₁-C₆ alkoxycarbonyl, and a saturated or unsaturated 3- to 10-membered ring system which may comprise at least one ring heteroatom selected from nitrogen, oxygen and sulphur, the ring system being optionally substituted by at least one substituent selected from halogen, hydroxyl, oxo, carboxyl, cyano, C₁-C₆ alkyl, C₁-C₆ hydroxyalkyl, —NR⁸R⁹, —(CH₂)ᵣNR¹⁰R¹¹ and —CONR¹²R¹³,
or R⁶ and R⁷ may together with the nitrogen atom to which they are attached form a saturated six-membered heterocyclic ring which may comprise a second ring heteroatom selected from nitrogen and oxygen, the ring being optionally substituted by at least one substituent selected from hydroxyl, halogen, C₁-C₆ alkyl and C₁-C₆ hydroxyalkyl;
r is 1, 2, 3, 4, 5 or 6;
R⁸ and R⁹ each independently represent a hydrogen atom or a C₁-C₆ alkyl, C₂-C₆ hydroxyalkyl or C₃-C₈ cycloalkyl group, or R⁸ and R⁹ together with the nitrogen atom to which they are attached form a 3- to 8-membered saturated heterocyclic ring;
R¹⁰ and R¹¹ each independently represent a hydrogen atom or a C₁-C₆ alkyl, C₂-C₆ hydroxyalkyl or C₃-C₈ cycloalkyl group, or R¹⁰ and R¹¹ together with the nitrogen atom to which they are attached form a 3- to 8-membered saturated heterocyclic ring; and
R¹² and R¹³ each independently represent a hydrogen atom or a C₁-C₆ alkyl, C₂-C₆ hydroxyalkyl or C₃-C₈ cycloalkyl group, or R¹² and R¹³ together with the nitrogen atom to which they are attached form a 3- to 8-membered saturated heterocyclic ring;
with the proviso that the compound of formula (I) is not N-(tricyclo[3.3.1.1^{3,7}]dec-1-ylmethyl)-2-quinolinecarboxamide, or 2-(2-thienyl)-N-(tricyclo[3.3.1.1^{3,7}]dec-1-ylmethyl)-4-quinolinecarboxamide;
or a pharmaceutically acceptable salt or solvate thereof.

2. A compound according to claim 1, wherein
m represents 1, 2 or 3;
each R¹ independently represents a hydrogen or halogen atom;
A represents C(O)NH or NHC(O);
Ar represents a group of formula 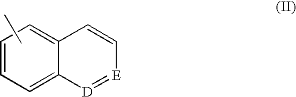
in which one of D and E represents a nitrogen atom and the other of D and E represents CH, the group of formula (II) being optionally substituted by one or more substituent groups R² independently selected from halogen, C₁-C₆ alkyl optionally substituted by at least one substituent selected from hydroxyl, halogen and C₁-C₆ alkoxy,
or a group of formula 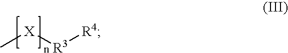
X represents an oxygen or sulphur atom or a group >N—R⁵;
n is 0 or 1;
R³ represents a bond or a C₁-C₅ alkyl group which may be optionally substituted by at least one substituent selected from hydroxyl, halogen, C₁-C₆ alkoxy, C₁-C₆ alkylthio, C₁-C₆ hydroxyalkyl, C₁-C₆ hydroxyalkyloxy, C₁-C₆ alkoxycarbonyl, C₃-C₈ cycloalkyl, phenyl (optionally substituted by at least one substituent selected from halogen, hydroxyl and C₁-C₆ alkylsulphonylamino), benzyl, indolyl (optionally substituted by at least one substituent selected from C₁-C₆ alkoxy), oxopyrrolidinyl, phenoxy, benzodioxolyl, phenoxyphenyl, piperidinyl and benzyloxy;
R⁴ represents hydrogen, hydroxyl or a group —NR⁶R⁷ except that when R³ represents a bond, then R⁴ represents a saturated or unsaturated 4- to 9-membered ring system which may comprise at least one ring heteroatom selected from nitrogen, oxygen and sulphur, the ring system being optionally substituted by at least one substituent selected from hydroxyl, C₁-C₆ hydroxyalkyl and benzyl;
R⁵ represents a hydrogen atom or a C₁-C₅ alkyl group which may be optionally substituted by at least one substituent selected from hydroxyl, halogen and C₁-C₆ alkoxy;
R⁶ and R⁷ each independently represent hydrogen, C₁-C₆alkylcarbonyl, C₂-C₇alkenyl, or C₁-C₇ alkyl optionally substituted with at least one substituent selected from carboxyl, hydroxyl, amino, C₁-C₆alkylamino, di-C₁-C₆alkylamino, C₁-C₆alkoxy, C₁-C₆ alkylthio, C₁-C₆ alkoxycarbonyl, and a saturated or unsaturated 3- to 10-membered ring system which may comprise at least one ring heteroatom selected from nitrogen, oxygen and sulphur, the ring system being optionally substituted by at least one substituent selected from halogen, hydroxyl, oxo, carboxyl, cyano, C₁-C₆ alkyl, C₁-C₆ hydroxyalkyl, —NR⁸R⁹, —(CH₂)ᵣNR¹⁰R¹¹ and —CONR¹²R¹³,
or R⁶ and R⁷ may together with the nitrogen atom to which they are attached form a saturated six-membered heterocyclic ring which may comprise a second ring heteroatom selected from nitrogen and oxygen, the ring being optionally substituted by at least one substituent selected from hydroxyl, halogen and C₁-C₆ alkyl;
r is 1, 2, 3, 4, 5 or 6;
R⁸ and R⁹ each independently represent a hydrogen atom or a C₁-C₆ alkyl, C₂-C₆ hydroxyalkyl or C₃-C₈ cycloalkyl group, or R⁸ and R⁹ together with the nitrogen atom to which they are attached form a 3- to 8-membered saturated heterocyclic ring;
R¹⁰ and R¹¹ each independently represent a hydrogen atom or a C₁-C₆ alkyl, C₂-C₆ hydroxyalkyl or C₃-C₈ cycloalkyl group, or R¹⁰ and R¹¹ together with the nitrogen atom to which they are attached form a 3- to 8-membered saturated heterocyclic ring; and
R¹² and R¹³ each independently represent a hydrogen atom or a C₁-C₆ allyl, C₂-C₆ hydroxyalkyl or C₃-C₈ cycloalkyl group, or R¹² and R¹³ together with the nitrogen atom to which they are attached form a 3- to 8-membered saturated heterocyclic ring.

3. A compound according to claim 1 or claim 2, wherein A represents C(O)NH.

4. A compound according to any one of claims 1 to 3, wherein the group of formula (II) bears a substituent R².

5. A compound according to claim 4, wherein R² represents a group of formula (III).

6. A compound according to claim 5, wherein n is 1 and X represents a group >N—R⁵.

7. A compound according to claim 5 or claim 6, wherein R⁴ represents a group —NR⁶R⁷.

8. A compound according to any one of the preceding claims, wherein Ar represents

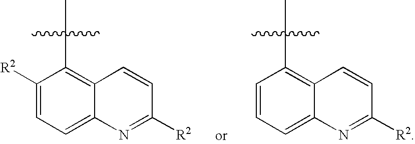

9. A compound according to claim 1 which is selected from the group consisting of:
2-(1-Adamantyl)-N-(4-methyl quinolin-5-yl)acetamide,
2-(1-Adamantyl)-N-(2-chloroquinolin-5-yl)acetamide,
2-(1-Adamantyl)-N-(6-methylquinolin-5-yl)acetamide,
2-(1-Adamantyl)-N-(2-chloro-6-methylquinolin-5-yl)acetamide,
2-(1-Adamantyl)-N-(6-chloroquinolin-5-yl)acetamide,
2-(1-Adamantyl)-N-{2-[(3-hydroxypropyl)amino]quinolin-5-yl}acetamide,
2-(1-Adamantyl)-N-(2-{[(2R)-2-hydroxypropyl]amino}quinolin-5-yl)acetamide,
2-(1-Adamantyl)-N-(2-{[(2S)-2-hydroxypropyl]amino}quinolin-5-yl)acetamide,
2-(1-Adamantyl)-N-{2-[(2-hydroxyethyl)amino]quinolin-5-yl}acetamide,
N-(1-Adamantyl)-N-(2-{[3-(4-methylpiperazin-1-yl)propyl]amino}quinolin-5-yl)acetamide,
2-(1-Adamantyl)-N-(2-{[(2S)-2,3-dihydroxypropyl]amino}quinolin-5-yl)acetamide,
2-(1-Adamantyl)-N-{2-[(3-hydroxypropyl)amino]-6-methylquinolin-5-yl}acetamide,
2-(1-Adamantyl)-N-{2-[(2-hydroxyethyl)amino]-6-methylquinolin-5-yl}acetamide,
2-(1-Adamantyl)-N-{2-[[2-(dimethylamino)ethyl](methyl)amino]-6-methylquinolin-5-yl}acetamide,
2-(1-Adamantyl)-N-{2-[(2-aminoethyl)amino]quinolin-5-yl}acetamide,
2-(1-Adamantyl)-N-{2-[(3-aminopropyl)amino]quinolin-5-yl}acetamide trifluoroacetate,
2-(1-Adamantyl)-N-[2-({2-[(2-hydroxyethyl)amino]ethyl}amino)quinolin-5-yl]acetamide dihydrochloride,
2-(1-Adamantyl)-N-{2-[(2-aminoethyl)(2-hydroxyethyl)amino]quinolin-5-yl}acetamide,
2-(1-Adamantyl)-N-[2-({2-[(cyclohex-3-en-1-ylmethyl)amino]ethyl}amino)quinolin-5-yl]acetamide,
2-(1-Adamantyl)-N-(2-{[2-(isobutylamino)ethyl]amino}quinolin-5-yl)acetamide,
2-(1-Adamantyl)-N-[2-({2-[(4-methylbenzyl)amino]ethyl}amino)quinolin-5-yl]acetamide,
{[2-({5-[(1-Adamantylacetyl)amino]quinolin-2-yl}amino)ethyl]amino}acetic acid,
2-(1-Adamantyl)-N-(2-{[2-(benzylamino)ethyl]amino}quinolin-5-yl)acetamide,
2-(1-Adamantyl)-N-(2-{[2-hexylamino)ethyl]amino}quinolin-5-yl)acetamide,
2-(1-Adamantyl)-N-(2-{[2-(propylamino)ethyl]amino}quinolin-5-yl)acetamide,
2-(1-Adamantyl)-N-(2-{[2-(heptylamino)ethyl]amino}quinolin-5-yl)acetamide,
2-(1-Adamantyl)-N-[2-({2-[(thien-2-ylmethyl)amino]ethyl}amino)quinolin-5-yl]acetamide,
2-(1-Adamantyl)-N-[2-({2-[(pyridin-2-ylmethyl)amino]ethyl}amino)quinolin-5-yl]acetamide,
2-(1-Adamantyl)-N-[2-({2-[(3-hydroxybenzyl)amino]ethyl}amino)quinolin-5-yl]acetamide,
2-(1-Adamantyl)-N-{2-[(2-{[(5-methyl-2-furyl)methyl]amino}ethyl)amino]quinolin-5-yl}acetamide,
2-(1-Adamantyl)-N-{2-[(2-{[(3-methylthien-2-yl)methyl]amino}ethyl)-amino]quinolin-5-yl}acetamide,
2-(1-Adamantyl)-N-[2-({2-[(thien-3-ylmethyl)amino]ethyl}amino)quinolin-5-yl]acetamide,
2-(1-Adamantyl)-N-(2-{[2-(pentylamino)ethyl]amino}quinolin-5-yl)acetamide,
2-(1-Adamantyl)-N-(2-{[2-(isopentylamino)ethyl]amino}quinolin-5-yl)acetamide,
2-(1-Adamantyl)-N-(2-{[2-(butylamino)ethyl]amino}quinolin-5-yl)acetamide,
2-(1-Adamantyl)-N-[2-({2-[(3,3-dimethylbutyl)amino]ethyl}amino)quinolin-5-yl]acetamide,
2-(1-Adamantyl)-N-[2-({2-[(bicyclo[2.2.1]hept-5-en-2-ylmethyl)amino]-ethyl}amino)quinolin-5-yl]acetamide,
2-(1-Adamantyl)-N-[2-({2-[(3-methylbenzyl)amino]ethyl}amino)quinolin-5-yl]acetamide,
2-(1-Adamantyl)-N-[2-({2-[(2-furylmethyl)amino]ethyl}amino)quinolin-5-yl]acetamide,
2-(1-Adamantyl)-N-[2-({2-[(4-fluorobenzyl)amino]ethyl}amino)quinolin-5-yl]acetamide,
2-(1-Adamantyl)-N-[2-({2-[(3-fluorobenzyl)amino]ethyl}amino)quinolin-5-yl]acetamide,
2-(1-Adamantyl)-N-[2-({2-[(3-furylmethyl)amino]ethyl}amino)quinolin-5-yl]acetamide,
2-(1-Adamantyl)-N-[2-({2-[(2-hydroxybenzyl)amino]ethyl}amino)quinolin-5-yl]acetamide,
2-(1-Adamantyl)-N-[2-({2-[(2E)-hex-2-enylamino]ethyl}amino)quinolin-5-yl]acetamide,
2-(1-Adamantyl)-N-[2-({2-[(2-fluorobenzyl)amino]ethyl}amino)quinolin-5-yl]acetamide,
2-(1-Adamantyl)-N-[2-({2-[(cyclopropylmethyl)amino]ethyl}amino)quinolin-5-yl]acetamide,
2-(1-Adamantyl)-N-[2-({2-[(5-hydroxypentyl)amino]ethyl}amino)quinolin-5-yl]acetamide,
2-(1-Adamantyl)-N-{2-[(2-{[(6-methylpyridin-2-yl)methyl]amino}ethyl)-amino]quinolin-5-yl}acetamide,
2-(1-Adamantyl)-N-[2-({2-[(2-methylbenzyl)amino]ethyl}amino)quinolin-5-yl]acetamide,
2-(1-Adamantyl)-N-[2-({2-[(2-phenylethyl)amino]ethyl}amino)quinolin-5-yl]acetamide,
2-(1-Adamantyl)-N-{2-[(2-{[(5-methylthien-2-yl)methyl]amino}ethyl)-amino]quinolin-5-yl}acetamide,
2-(1-Adamantyl)-N-(2-{[2-({[5-(hydroxymethyl)-2-furyl]methyl}amino)-ethyl]amino}quinolin-5-yl)acetamide,
2-(1-Adamantyl)-N-{2-[(2-{[3-(methylthio)propyl]amino}ethyl)amino]quinolin-5-yl}acetamide,
2-(1-Adamantyl)-N-[2-({2-[(3,4-dihydro-2H-pyran-5-ylmethyl)amino]-ethyl}amino)quinolin-5-yl]acetamide,
2-(1-Adamantyl)-N-[2-({2-[(1,3-thiazol-2-ylmethyl)amino]ethyl}amino)quinolin-5-yl]acetamide,
2-(1-Adamantyl)-N-[2-({2-[(3-hydroxy-2,2-dimethylpropyl)amino]-ethyl}amino)quinolin-5-yl]acetamide,
2-(1-Adamantyl)-N-{2-[(2-{[3-(methylthio)butyl]amino}ethyl)amino]quinolin-5-yl}acetamide,
2-(1-Adamantyl)-N-[2-({2-[(2-ethylbutyl)amino]ethyl}amino)quinolin-5-yl]acetamide,
2-(1-Adamantyl)-N-{2-[(2-{[(2E)-2-methylbut-2-enyl]amino}ethyl)amino]quinolin-5-yl}acetamide,
2-(1-Adamantyl)-N-{2-[(2-{[(2E)-2-methylpent-2-enyl]amino}ethyl)amino]quinolin-5-yl}acetamide,
2-(1-Adamantyl)-N-{2-[(2-{[(1-methyl-1H-pyrrol-2-yl)methyl]amino}ethyl)amino]-quinolin-5-yl}acetamide,
2-(1-Adamantyl)-N-{2-[(2-{[(1-oxidopyridin-4-yl)methyl]amino}ethyl)-amino]quinolin-5-yl}acetamide,
2-(1-Adamantyl)-N-[2-({2-[(2-ethyl-3-methylbutyl)amino]ethyl}amino)quinolin-5-yl]acetamide,
2-(1-Adamantyl)-N-[2-({2-[(1H-pyrazol-3-ylmethyl)amino]ethyl}amino)quinolin-5-yl]acetamide,
Ethyl {[2-({5-[(1-adamantylacetyl)amino]quinolin-2-yl}amino)ethyl]amino}acetate,
2-(1-Adamantyl)-N-[2-({2-[(2,2-dimethylpent-4-enyl)amino]ethyl}amino)quinolin-5-yl]acetamide,
2-(1-Adamantyl)-N-{2-[(2-{[(1-methyl-1H-imidazol-2-yl)methyl]amino}ethyl)-amino]quinolin-5-yl}acetamide,
2-(1-Adamantyl)-N-{2-[(2-{[(2-ethyl-1H-imidazol-5-yl)methyl]amino}ethyl)amino]-quinolin-5-yl}acetamide,
2-(1-Adamantyl)-N-[2-({2-[(1,2,3-thiadiazol-4-ylmethyl)amino]ethyl}amino)quinolin-5-yl]acetamide,
2-(1-Adamantyl)-N-[2-({3-[(cyclohex-3-en-1-ylmethyl)amino]propyl}amino)quinolin-5-yl]acetamide,
2-(1-Adamantyl)-N-(2-{[3-(isobutylamino)propyl]amino}quinolin-5-yl)acetamide,
2-(1-Adamantyl)-N-[2-({3-[(4-methylbenzyl)amino]propyl}amino)quinolin-5-yl]acetamide,
{[3-({5-[(1-Adamantylacetyl)amino]quinolin-2-yl}amino)propyl]amino}acetic acid,
2-(1-Adamantyl)-N-(2-{[3-(benzylamino)propyl]amino}quinolin-5-yl)acetamide,
2-(1-Adamantyl)-N-(2-{[3-(hexylamino)propyl]amino}quinolin-5-yl)acetamide,
2-(1-Adamantyl)-N-(2-{[3-(propylamino)propyl]amino}quinolin-5-yl)acetamide,
2-(1-Adamantyl)-N-(2-{[3-(heptylamino)propyl]amino}quinolin-5-yl)acetamide,
2-(1-Adamantyl)-N-[2-({3-[(thien-2-ylmethyl)amino]propyl}amino)quinolin-5-yl]acetamide,
2-(1-Adamantyl)-N-[2-({3-[(pyridin-2-ylmethyl)amino]propyl}amino)quinolin-5-yl]acetamide,
2-(1-Adamantyl)-N-[2-({3-[(3-hydroxybenzyl)amino]propyl}amino)quinolin-5-yl]acetamide,
2-(1-Adamantyl)-N-{2-[(3-{[(5-methyl-2-furyl)methyl]amino}propyl)amino]quinolin-5-yl}acetamide,
2-(1-Adamantyl)-N-{2-[(3-{[(3-methylthien-2-yl)methyl]amino}propyl)amino]-quinolin-5-yl}acetamide,
2-(1-Adamantyl)-N-[2-({3-[(thien-3-ylmethyl)amino]propyl}amino)quinolin-5-yl]acetamide,
2-(1-Adamantyl)-N-(2-{[3-(pentylamino)propyl]amino}quinolin-5-yl)acetamide,
2-(1-Adamantyl)-N-(2-{[3-(isopentylamino)propyl]amino}quinolin-5-yl)acetamide,
2-(1-Adamantyl)-N-(2-{[3-(butylamino)propyl]amino}quinolin-5-yl)acetamide,
2-(1-Adamantyl)-N-[2-({3-[(3,3-dimethylbutyl)amino]propyl}amino)quinolin-5-yl]acetamide,
2-(1-Adamantyl)-N-[2-({3-[(bicyclo[2.2.1]hept-5-en-2-ylmethyl)amino]propyl}-amino)quinolin-5-yl]acetamide,
2-(1-Adamantyl)-N-[2-({3-[(3-methylbenzyl)amino]propyl}amino)quinolin-5-yl]acetamide,
2-(1-Adamantyl)-N-[2-({3-[(2-furylmethyl)amino]propyl}amino)quinolin-5-yl]acetamide,
2-(1-Adamantyl)-N-[2-({3-[(4-fluorobenzyl)amino]propyl}amino)quinolin-5-yl]acetamide,
2-(1-Adamantyl)-N-[2-({3-[(3-fluorobenzyl)amino]propyl}amino)quinolin-5-yl]acetamide,
2-(1-Adamantyl)-N-[2-({3-[(3-furylmethyl)amino]propyl}amino)quinolin-5-yl]acetamide,
2-(1-Adamantyl)-N-[2-({3-[(2-hydroxybenzyl)amino]propyl}amino)quinolin-5-yl]acetamide,
2-(1-Adamantyl)-N-[2-({3-[(2E)-hex-2-enylamino]propyl}amino)quinolin-5-yl]acetamide,
2-(1-Adamantyl)-N-[2-({3-[(2-fluorobenzyl)amino]propyl}amino)quinolin-5-yl]acetamide,
2-(1-Adamantyl)-N-[2-({3-[(cyclopropylmethyl)amino]propyl}amino)quinolin-5-yl]acetamide,
2-(1-Adamantyl)-N-[2-({3-[(1H-imidazol-2-ylmethyl)amino]propyl}amino)quinolin-5-yl]acetamide,
2-(1-Adamantyl)-N-[2-({3-[(5-hydroxypentyl)amino]propyl}amino)quinolin-5-yl]acetamide,
2-(1-Adamantyl)-N-{2-[(3-{[(6-methylpyridin-2-yl)methyl]amino}propyl)amino]-quinolin-5-yl}acetamide,
2-(1-Adamantyl)-N-[2-({3-[(2-methylbenzyl)amino]propyl}amino)quinolin-5-yl]acetamide,
2-(1-Adamantyl)-N-[2-({3-[(2-phenylethyl)amino]propyl}amino)quinolin-5-yl]acetamide,
2-(1-Adamantyl)-N-{2-[(3-{[(5-ethyl-2-furyl)methyl]amino}propyl)amino]quinolin-5-yl}acetamide,
2-(1-Adamantyl)-N-{2-[(3-{[(5-methylthien-2-yl)methyl]amino}propyl)amino]-quinolin-5-yl}acetamide,
2-(1-Adamantyl)-N-{2-[(3-{[3-(methylthio)propyl]amino}propyl)amino]quinolin-5-yl}acetamide,
2-(1-Adamantyl)-N-[2-({3-[(3,4-dihydro-2H-pyran-5-ylmethyl)amino]propyl}amino)-quinolin-5-yl]acetamide,
2-(1-Adamantyl)-N-[2-({3-[(1,3-thiazol-2-ylmethyl)amino]propyl}amino)quinolin-5-yl]acetamide,
2-(1-Adamantyl)-N-[2-({3-[(3-hydroxy-2,2-dimethylpropyl)amino]propyl}amino)-quinolin-5-yl]acetamide,
2-(1-Adamantyl)-N-{2-[(3-{[3-(methylthio)butyl]amino}propyl)amino]quinolin-5-yl}acetamide,
2-(1-Adamantyl)-N-{2-[(3-{[3-(dimethylamino)-2,2-dimethylpropyl]-amino}propyl)amino]quinolin-5-yl}acetamide,
2-(1-Adamantyl)-N-[2-({3-[(2-ethylbutyl)amino]propyl}amino)quinolin-5-yl]acetamide,
2-(1-Adamantyl)-N-{2-[(3-{[(2E)-2-methylbut-2-enyl]amino}propyl)amino]quinolin-5-yl}acetamide,
2-(1-Adamantyl)-N-{2-[(3-{[(2E)-2-methylpent-2-enyl]amino}propyl)-amino]quinolin-5-yl}acetamide,
2-(1-Adamantyl)-N-{2-[(3-{[(1-methyl-1H-pyrrol-2-yl)methyl]amino}propyl)amino]-quinolin-5-yl}acetamide,
2-(1-Adamantyl)-N-[2-({3-[(2-ethyl-3-methylbutyl)amino]propyl}amino)quinolin-5-yl]acetamide,
Ethyl {[3-({5-[(1-adamantylacetyl)amino]quinolin-2-yl}amino)propyl]amino}acetate,
2-(1-Adamantyl)-N-[2-({3-[(2,2-dimethylpent-4-enyl)amino]propyl}amino)quinolin-5-yl]acetamide,
2-(1-Adamantyl)-N-[2-({3-[(1,2,3-thiadiazol-4-ylmethyl)amino]propyl}amino)-quinolin-5-yl]acetamide,
2-(1-Adamantyl)-N-{2-[(4-hydroxybutyl)amino]quinolin-5-yl}acetamide,
Methyl 3-({5-[(1-adamantylacetyl)amino]quinolin-2-yl}amino)propanoate,
N-(2-{[2-(Acetylamino)ethyl]amino}quinolin-5-yl)-2-(1-adamantyl)acetamide,
2-(1-Adamantyl)-N-{2-[(1-benzyl-2-hydroxyethyl)amino]quinolin-5-yl}acetamide,
2-(1-Adamantyl)-N-(2-{[1-(hydroxymethyl)propyl]amino}quinolin-5-yl)acetamide,
2-(1-Adamantyl)-N-(2-{[(2S)-2-hydroxycyclohexyl]amino}quinolin-5-yl)acetamide,
2-(1-Adamantyl)-N-{2-[(2-morpholin-4-ylethyl)amino]quinolin-5-yl}acetamide,
2-(1-Adamantyl)-N-{2-[(2-hydroxy-2-phenylethyl)amino]quinolin-5-yl}acetamide,
2-(1-Adamantyl)-N-{2-[(2-hydroxy-1-methylethyl)amino]quinolin-5-yl}acetamide,
2-(1-Adamantyl)-N-{2-[(2-methoxyethyl)amino]quinolin-5-yl}acetamide,
2-(1-Adamantyl)-N-(2-{[2-(5-methoxy-1H-indol-3-yl)ethyl]amino}quinolin-5-yl)acetamide,
2-(1-Adamantyl)-N-(2-{[2-(4-hydroxyphenyl)ethyl]amino}quinolin-5-yl)acetamide,
2-(1-Adamantyl)-N-{2-[(2-hydroxy-1-phenylethyl)amino]quinolin-5-yl}acetamide,
2-(1-Adamantyl)-N-(2-{[1-(hydroxymethyl)-3-methylbutyl]amino}quinolin-5-yl)-acetamide,
2-(1-Adamantyl)-N-[2-(isobutylamino)quinolin-5-yl]acetamide,
2-(1-Adamantyl)-N-(2-{[1-(hydroxymethyl)propyl]amino}quinolin-5-yl)acetamide,
2-(1-Adamantyl)-N-{2-[(3-ethoxypropyl)amino]quinolin-5-yl}acetamide,
2-(1-Adamantyl)-N-{2-[(2-hydroxy-2,3-dihydro-1H-inden-1-yl)amino]quinolin-5-yl}acetamide,
2-(1-Adamantyl)-N-(2-{[2-(2-hydroxyethoxy)ethyl]amino}quinolin-5-yl)acetamide,
2-(1-Adamantyl)-N-[2-(cyclobutylamino)quinolin-5-yl]acetamide,
2-(1-Adamantyl)-N-(2-{[3-(2-oxopyrrolidin-1-yl)propyl]amino}quinolin-5-yl)-acetamide,
2-(1-Adamantyl)-N-{2-[(1-benzylpyrrolidin-3-yl)amino]quinolin-5-yl}acetamide,
2-(1-Adamantyl)-N-(2-{[2-(methylthio)ethyl]amino}quinolin-5-yl)acetamide,
2-(1-Adamantyl)-N-{2-[(3-methoxypropyl)amino]quinolin-5-yl}acetamide,
2-(1-Adamantyl)-N-{2-[(2-phenoxyethyl)amino]quinolin-5-yl}acetamide,
2-(1-Adamantyl)-N-(2-{[2-(1,3-benzodioxol-5-yl)ethyl]amino}quinolin-5-yl)-acetamide,
2-(1-Adamantyl)-N-(2-{[2-(4-phenoxyphenyl)ethyl]amino}quinolin-5-yl)acetamide,
2-(1-Adamantyl)-N-(2-{[2-(1H-indol-3-yl)ethyl]amino}quinolin-5-yl)acetamide,
2-(1-Adamantyl)-N-{2-[(2-piperidin-1-ylethyl)amino]quinolin-5-yl}acetamide,
2-(1-Adamantyl)-N-(2-{[2-hydroxy-1-(hydroxymethyl)ethyl]amino}quinolin-5-yl)acetamide,
2-(1-Adamantyl)-N-(2-{[(1R)-1-(hydroxymethyl)-2,2-dimethylpropyl]-amino}quinolin-5-yl)acetamide,
2-(1-Adamantyl)-N-(2-{[2-(3-hydroxyphenyl)ethyl]amino}quinolin-5-yl)acetamide,
2-(1-Adamantyl)-N-(2-{[(1S,3R,4R)-3-(hydroxymethyl)bicyclo[2.2.1]hept-2-yl]amino}quinolin-5-yl)acetamide,
2-(1-Adamantyl)-N-(2-{[(1R,3R,4S)-3-(hydroxymethyl)bicyclo[2.2.1]hept-2-yl]amino}quinolin-5-yl)acetamide,
2-(1-Adamantyl)-N-(2-{[2-(benzyloxy)-1-(hydroxymethyl)ethyl]amino}quinolin-5-yl)acetamide,
2-(1-Adamantyl)-N-{2-[(cyclopropylmethyl)amino]quinolin-5-yl}acetamide,
2-(1-Adamantyl)-N-(2-{[2-(4-chlorophenyl)-1-methylethyl]amino}quinolin-5-yl)-acetamide,
2-(1-Adamantyl)-N-(2-{[1-(hydroxymethyl)propyl]amino}quinolin-5-yl)acetamide,
2-(1-Adamantyl)-N-{2-[(2-{4-[(methylsulfonyl)amino]phenyl}ethyl)amino]quinolin-5-yl}acetamide,
2-(1-Adamantyl)-N-[2-({2-[bis(2-hydroxyethyl)amino]ethyl}amino)quinolin-5-yl]acetamide,
2-(1-Adamantyl)-N-quinolin-5-ylacetamide,
2-(1-Adamantyl)-N-isoquinolin-5-ylacetamide,
2-(1-Adamantyl)-N-[2-(3-{[(1R)-2-hydroxy-1-methylethyl]amino}propyl)quinolin-5-yl]acetamide dihydrochloride,
2-(1-Adamantyl)-N-(2-{2-[benzyl(2-hydroxyethyl)amino]ethoxy}quinolin-5-yl)acetamide,
2-(1-Adamantyl)-N-(2-{2-[(2-hydroxyethyl)amino]ethoxy}quinolin-5-yl)acetamide,
2-(1-Adamantyl)-N-{2-[bis(2-hydroxyethyl)amino]quinolin-5-yl}acetamide,
2-(1-Adamantyl)-N-[8-({2-[(2-hydroxyethyl)amino]ethyl}amino)quinolin-5-yl]acetamide trihydrochloride,
2-(1-Adamantyl)-N-{8-[(2-aminoethyl)thio]quinolin-5-yl}acetamide,
N-(1-Adamantylmethyl)-6-chloro-2-[3-(methylamino)propyl]quinoline-5-carboxamide sesquihydrochloride dihydrate,
N-(1-Adamantylmethyl)-2-{3-[(3-hydroxypropyl)amino]propyl}quinoline-4-carboxamide benzoic acid salt,
N-(1-Adamantylmethyl)-8-[3-(methylamino)propyl]quinoline-4-carboxamide dihydrochloride,
N-(1-Adamantylmethyl)-6-chloro-2-(piperazin-1-ylmethyl)quinoline-5-carboxamide hydrochloride,
N-(1-Adamantylmethyl)-quinoline-5-carboxamide trifluoroacetate,
N-(1-Adamantylmethyl)-2-{3-[(3-hydroxypropyl)amino]propyl}quinoline-5-carboxamide dihydrochloride,
N-(1-adamantylmethyl)-2-[3-(ethylamino)propyl]quinoline-5-carboxamide dihydrochloride,
2-(1-Adamantyl)-N-[2-({2-[(2-hydroxyethyl)amino]ethyl}amino)-6-methylquinolin-5-yl]acetamide hydrochloride,
2-(1-Adamantyl)-N-[2-({2-[(2-hydroxyethyl)amino]ethyl}amino)-6-chloroquinolin-5-yl]acetamide dihydrochloride,
2-(1-Adamantyl)-N-[2-({2-[(2-hydroxyethyl)amino]ethyl}amino)quinolin-5-yl]acetamide dihydrochloride,
2-(1-Adamantyl)-N-(2-{3-[(3-hydroxypropyl)amino]propyl}quinolin-5-yl)acetamide dihydrochloride,
2-(1-Adamantyl)-N-(6-methyl-2-piperazin-1-ylquinolin-5-yl)acetamide dihydrochloride,
2-(1-Adamantyl)-N-{2-[4-(2-hydroxyethyl)piperazin-1-yl]-6-methylquinolin-5-yl}acetamide dihydrochloride,
2-(1-Adamantyl)-N-[2-(4-aminopiperidin-1-yl)-6-methylquinolin-5-yl]acetamide dihydrochloride,
2-(1-Adamantyl)-N-(2-{4-[(2-hydroxyethyl)amino]piperidin-1-yl}-6-methylquinolin-5-yl)acetamide dihydrochloride,
2-(1-Adamantyl)-N-{2-[(3S)-3-aminopyrrolidin-1-yl]-6-methylquinolin-5-yl}acetamide dihydrochloride,
(3S)—N-((3S)-1-{5-[(1-Adamantylacetyl)amino]-6-methylquinolin-2-yl}pyrrolidin-3-yl)-3-aminopyrrolidine-1-carboxamide dihydrochloride,
2-(1-Adamantyl)-N-{6-methyl-2-[(1-methylpiperidin-4-yl)amino]quinolin-5-yl}acetamide dihydrochloride,
2-(1-Adamantyl)-N-{6-methyl-2-[(3S)-3-(methylamino)pyrrolidin-1-yl]quinolin-5-yl}acetamide dihydrochloride,
2-(1-Adamantyl)-N-{2-[(3S)-3-(ethylamino)pyrrolidin-1-yl]-6-methylquinolin-5-yl}acetamide dihydrochloride,
2-(1-Adamantyl)-N-(2-{(3S)-3-[(2-hydroxyethyl)amino]pyrrolidin-1-yl}-6-methylquinolin-5-yl)acetamide dihydrochloride,
2-(1-Adamantyl)-N-(2-{(3S)-3-[(3-hydroxypropyl)amino]pyrrolidin-1-yl}-6-methylquinolin-5-yl)acetamide dihydrochloride,
2-(1-Adamantyl)-N-{6-chloro-2-[(3R)-3,4-dihydroxybutyl]quinolin-5-yl}acetamide hydrochloride,
2-(1-Adamantyl)-N-{6-chloro-2-[(3R)-3-hydroxy-4-(methylamino)butyl]quinolin-5-yl}acetamide dihydrochloride,
2-(1-Adamantyl)-N-{2-[(3R)-3-aminopyrrolidin-1-yl]-6-methylquinolin-5-yl}acetamide dihydrochloride,
2-(1-Adamantyl)-N-(6-chloro-2-{[2-(methylamino)ethyl]amino}quinolin-5-yl)acetamide dihydrochloride,
2-(1-Adamantyl)-N-(6-chloro-2-{methyl[3-(methylamino)propyl]amino}quinolin-5-yl)acetamide dihydrochloride,
2-(1-Adamantyl)-N-(6-chloro-2-{3-[(3-hydroxypropyl)amino]propyl}quinolin-5-yl)acetamide dihydrochloride,
2-(1-Adamantyl)-N-[6-chloro-2-({3-[(2-hydroxyethyl)amino]propyl}amino)quinolin-5-yl]acetamide dihydrochloride,
2-(1-Adamantyl)-N-(6-chloro-2-{[4-(2-hydroxyethyl)piperazin-1-yl]methyl}quinolin-5-yl)acetamide,
2-(1-Adamantyl)-N-[6-chloro-2-({[2-(methylamino)ethyl]amino}methyl)quinolin-5-yl]acetamide,
2-(1-Adamantyl)-N-{6-chloro-2-[({2-[(2-hydroxyethyl)amino]ethyl}-amino)methyl]quinolin-5-yl}acetamide,
2-(1-Adamantyl)-N-[6-chloro-2-({[3-(methylamino)propyl]amino}methyl)quinolin-5-yl]acetamide bis(trifluoroacetate),
2-(1-Adamantyl)-N-(6-chloro-2-{[(3R)-pyrrolidin-3-ylamino]methyl}quinolin-5-yl)acetamide tris(trifluoroacetate),
2-(1-Adamantyl)-N-[2-({3-[(pyridin-2-ylmethyl)amino]propyl}amino)quinolin-5-yl]acetamide,
2-(1-Adamantyl)-N-[2-({2-[(2-hydroxyethyl)amino]propyl}amino)-6-methylquinolin-5-yl]acetamide hydrochloride,
N-(1-Adamantylmethyl)-2-[3-(methylamino)propyl]quinoline-5-carboxamide dihydrochloride,
2-(1-Adamantyl)-N-(6-methyl-2-{[3-(methylamino)propyl]amino}quinolin-5-yl)acetamide,
2-(1-Adamantyl)-N-(2-{2-[(3-hydroxypropyl)amino]ethyl}-6-methylquinolin-5-yl)acetamide dihydrochloride,
2-(1-Adamantyl)-N-[6-chloro-2-(piperazin-1-ylmethyl)quinolin-5-yl]acetamide trifluoroacetate,
2-(1-Adamantyl)-N-(6-chloro-2-piperazin-1-ylquinolin-5-yl)acetamide, and
N-(1-Adamantylmethyl)-6-chloro-2-{methyl[3-(methylamino)propyl]amino}quinoline-5-carboxamide.

10. A process for the preparation of a compound of formula (I) as defined in claim 1 which comprises:
(a) reacting a compound of formula 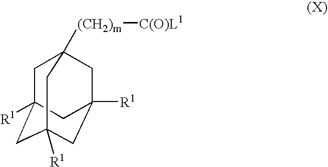
wherein L¹ represents a leaving group and m and R¹ are as defined in formula (I), with a compound of formula (XI), Ar—NH₂, wherein Ar is as defined in formula (I); or
(b) reacting a compound of formula 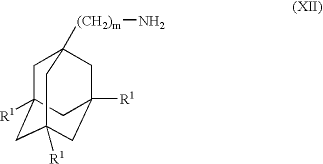
wherein m and R¹ are as defined in formula (I), with a compound of formula (XIII), Ar—C(O)-L², wherein L² represents a leaving group and Ar is as defined in formula (I; or
(c) when Ar represents a group 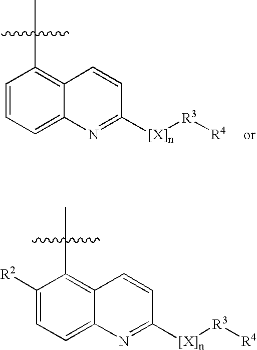
in which n is 1, X is >N—R⁵ and R² is other than a group of formula (III),
reacting a compound of formula 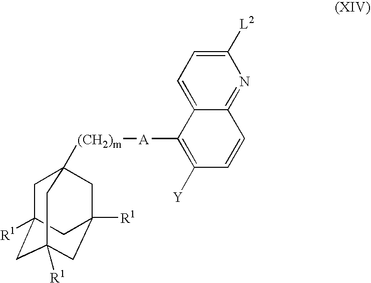
wherein L² is a leaving group, Y is hydrogen or a group R^{2a} which represents halogen or C₁-C₆ alkyl optionally substituted by at least one substituent selected from hydroxyl, halogen and C₁-C₆ alkoxy, and m, A and R¹ are as defined in formula (I), with a compound of formula (XV), H—N(R⁵)-R³-R⁴, wherein R³R⁴ and R⁵ are as defined in formula (I); or
(d) when Ar represents a group 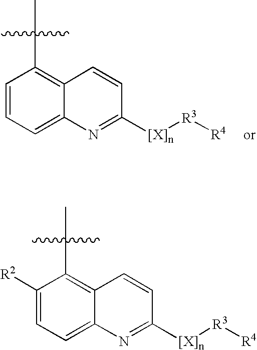
in which n is 0, R² is other than a group of formula (III) and R³ is an optionally substituted C₃-C₅ alkyl group, reacting a compound of formula (XIV) as defined in (c) above with a compound of formula 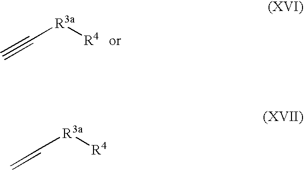
wherein R^{3a} represents a C₁-C₃ alkyl group optionally substituted as defined for R³ in formula (I) and R⁴ is as defined in formula (I), optionally followed by a hydrogenation reaction; or
(e) when Ar represents a group 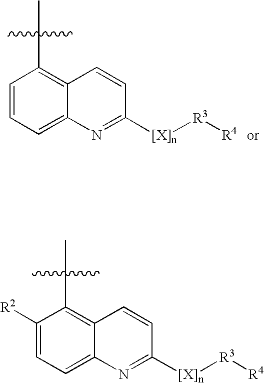
in which n is 0, R² is other than a group of formula (III), R³ is (CH₂)₂ and R⁴ is —NR⁶R⁷, reacting a compound of formula (XIV) as defined in (c) above with a compound of formula 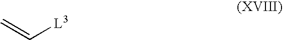
wherein L³ is a leaving group, followed by reaction with a compound of formula (XIX), HNR⁶R⁷, wherein R⁶ and R⁷ are as defined in formula (I); or
(f) when Ar represents a group 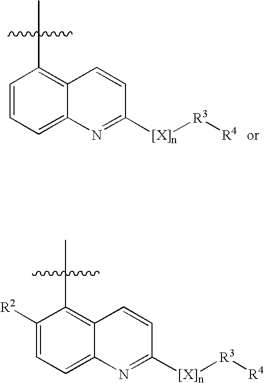
in which n is 0, R² is other than a group of formula (III), R³ is CH₂ and R⁴ is —NR⁶R⁷, reacting a compound of formula (XIV) as defined in (c) above with a compound of formula (XVIII) as defined in (e) above, followed by an oxidation reaction and then by reaction with a compound of formula (XIX) as defined in (e) above under reductive amination conditions;
and optionally after (a), (b), (c), (d), (e) or (f) carrying out one or more of the following:
converting the compound of formula (I) obtained to a further compound of formula (I)
forming a pharmaceutically acceptable salt or solvate of the compound of formula (I).

11. A pharmaceutical composition comprising a compound of formula (I), or a pharmaceutically acceptable salt or solvate thereof, as claimed in any one of claims 1 to 9 in association with a pharmaceutically acceptable adjuvant, diluent or carrier.

12. A process for the preparation of a pharmaceutical composition as claimed in claim 11 which comprises mixing a compound of formula (I), or a pharmaceutically acceptable salt or solvate thereof, as defined in any one of claims 1 to 9 with a pharmaceutically acceptable adjuvant, diluent or carrier.

13. A compound of formula (I), or a pharmaceutically acceptable salt or solvate thereof, as claimed in any one of claims 1 to 9 for use in therapy.

14. Use of a compound of formula (I), or a pharmaceutically acceptable salt or solvate thereof, as claimed in any one of claims 1 to 9 in the manufacture of a medicament for use in the treatment of rheumatoid arthritis.

15. Use of a compound of formula (I) or a pharmaceutically acceptable salt or solvate thereof as claimed in any one of claims 1 to 9 in the manufacture of a medicament for use in the treatment of an obstructive airways disease.

16. Use according to claim 15, wherein the obstructive airways disease is asthma or chronic obstructive pulmonary disease.

17. Use of a compound of formula (I) or a pharmaceutically acceptable salt or solvate thereof as claimed in any one of claims 1 to 9 in the manufacture of a medicament for use in the treatment of osteoarthritis.

18. Use of a compound of formula (I) or a pharmaceutically acceptable salt or solvate thereof as claimed in any one of claims 1 to 9 in the manufacture of a medicament for use in the treatment of atherosclerosis.

19. A method of treating rheumatoid arthritis or osteoarthritis which comprises administering to a patient a therapeutically effective amount of a compound of formula (I) or a pharmaceutically acceptable salt or solvate thereof as claimed in any one of claims 1 to 9.

20. A method of treating an obstructive airways disease which comprises administering to a patient a therapeutically effective amount of a compound of formula (I) or a pharmaceutically acceptable salt or solvate thereof as claimed in any one of claims 1 to 9.
